SEQUENCE LISTING (278 sequences)

SEQ ID NO: 1 - DNA, 12 bp, Artificial Sequence
  Synthetic
ggggcatgcccc

SEQ ID NO: 2 - DNA, 12 bp, Artificial Sequence
  Synthetic
gagaggcctctc

SEQ ID NO: 3 - protein, 10 aa, Artificial Sequence
  Synthetic
RKRGTAGVAG

SEQ ID NO: 4 - protein, 11 aa, Artificial Sequence
  Synthetic
  REPEAT (1)..(1): G repeats
GRKRGTAGVAG

SEQ ID NO: 5 - protein, 33 aa, Artificial Sequence
  Synthetic
RGLFVGCFFNFFNPFSCTVFFLVSGAGETPARY

SEQ ID NO: 6 - protein, 34 aa, Artificial Sequence
  Synthetic
  REPEAT (1)..(1): P repeats
PRGLFVGCFFNFFNPFSCTVFFLVSGAGETPARY

SEQ ID NO: 7 - protein, 21 aa, Artificial Sequence
  Synthetic
GVGGESAGLPEWQDDVMRMSV

SEQ ID NO: 8 - protein, 23 aa, Artificial Sequence
  Synthetic
  REPEAT (1)..(2): GA repeats
GAGVGGESAGLPEWQDDVMRMSV

SEQ ID NO: 9 - protein, 15 aa, Artificial Sequence
  Synthetic
GWGEKARDCRSGRMM

SEQ ID NO: 10 - protein, 17 aa, Artificial Sequence
  Synthetic
  REPEAT (1)..(2): GR repeats
GRGWGEKARDCRSGRMM

SEQ ID NO: 11 - protein, 43 aa, Artificial Sequence
  Synthetic
PVACLLVVFLIFLTPFLVLSSFWCQGLERLLQDIEAFRMYGSV

SEQ ID NO: 12 - protein, 45 aa, Artificial Sequence
  Synthetic
  REPEAT (1)..(2): PR repeats
PRPVACLLVVFLIFLTPFLVLSSFWCQGLERLLQDIEAFRMYGSV

SEQ ID NO: 13 - protein, 9 aa, Artificial Sequence
  Synthetic
PWLVCWLFF

SEQ ID NO: 14 - protein, 11 aa, Artificial Sequence
  Synthetic
  REPEAT (1)..(2): PA repeats
PAPWLVCWLFF

SEQ ID NO: 15 - protein, 42 aa, Artificial Sequence
  Synthetic
GGRRREEVVLIPHFWLPEKALWQRDAGASKLKVQSACTEGKN

SEQ ID NO: 16 - protein, 44 aa, Artificial Sequence
  Synthetic
  REPEAT (1)..(2): GA repeats
GAGGRRREEVVLIPHFWLPEKALWQRDAGASKLKVQSACTEGKN

SEQ ID NO: 17 - protein, 9 aa, Artificial Sequence
  Synthetic
GAGGEKRWY

SEQ ID NO: 18 - protein, 11 aa, Artificial Sequence
  Synthetic
  REPEAT (1)..(2): GQ repeats
GQGAGGEKRWY

SEQ ID NO: 19 - protein, 27 aa, Artificial Sequence
  Synthetic
GQEERRGGINSPFLASRESPLAKRCRC

SEQ ID NO: 20 - protein, 29 aa, Artificial Sequence
  Synthetic
  REPEAT (1)..(2): GR repeats
GRGQEERRGGINSPFLASRESPLAKRCRC

SEQ ID NO: 21 - DNA, 24 bp, Artificial Sequence
  Synthetic
tttactcccctctccctcccggtg

SEQ ID NO: 22 - protein, 31 aa, Artificial Sequence
  Synthetic
PSSPQLITHGSWCINTSASLPTRKEDGVEYL

SEQ ID NO: 23 - protein, 33 aa, Artificial Sequence
  Synthetic
  REPEAT (1)..(2): PC repeats
PCPSSPQLITHGSWCINTSASLPTRKEDGVEYL

SEQ ID NO: 24 - protein, 6 aa, Artificial Sequence
  Synthetic
PVVLSS

SEQ ID NO: 25 - protein, 8 aa, Artificial Sequence
  Synthetic
  REPEAT (1)..(2): PA repeats
PAPVVLSS

SEQ ID NO: 26 - protein, 2 aa, Artificial Sequence
  Synthetic
  REPEAT (1)..(2): PL repeats
PL

SEQ ID NO: 27 - protein, 2 aa, Artificial Sequence
  Synthetic
  REPEAT (1)..(2): GR repeats
GR

SEQ ID NO: 28 - protein, 18 aa, Artificial Sequence
  Synthetic
GDSNTTSAKSQDTASLQM

SEQ ID NO: 29 - protein, 20 aa, Artificial Sequence
  Synthetic
  REPEAT (1)..(2): GA repeats
GAGDSNTTSAKSQDTASLQM

SEQ ID NO: 30 - protein, 26 aa, Artificial Sequence
  Synthetic
GTVTQHLPRVKTQPLCKCRQAMLRCV

SEQ ID NO: 31 - protein, 28 aa, Artificial Sequence
  Synthetic
  REPEAT (1)..(2): GQ repeats
GQGTVTQHLPRVKTQPLCKCRQAMLRCV

SEQ ID NO: 32 - protein, 2 aa, Artificial Sequence
  Synthetic
  REPEAT (1)..(2): PA repeats
PA

SEQ ID NO: 33 - protein, 27 aa, Artificial Sequence
  Synthetic
PSHNSLTLVSSLTLPLDTIGTDPQQSA

SEQ ID NO: 34 - protein, 29 aa, Artificial Sequence
  Synthetic
  REPEAT (1)..(2): PL repeats
PLPSHNSLTLVSSLTLPLDTIGTDPQQSA

SEQ ID NO: 35 - protein, 7 aa, Artificial Sequence
  Synthetic
  REPEAT (1)..(2): PC repeats
PCPHIIL

SEQ ID NO: 36 - protein, 20 aa, Artificial Sequence
  Synthetic
PGHSSSSTTIATTPGRSLPM

SEQ ID NO: 37 - protein, 22 aa, Artificial Sequence
  Synthetic
  REPEAT (1)..(2): PA repeats
PAPGHSSSSTTIATTPGRSLPM

SEQ ID NO: 38 - protein, 36 aa, Artificial Sequence
  Synthetic
GTHRPAQQLQQHLGVLCPCSEVKVLGAELSLDVQSF

SEQ ID NO: 39 - protein, 37 aa, Artificial Sequence
  Synthetic
  REPEAT (1)..(1): P repeats
PGTHRPAQQLQQHLGVLCPCSEVKVLGAELSLDVQSF

SEQ ID NO: 40 - protein, 53 aa, Artificial Sequence
  Synthetic
ALIVQHNNCNNTWAFSAHVARSRSWEPNSPLMFNLFKSFPAFISHLQNDDNRI

SEQ ID NO: 41 - protein, 55 aa, Artificial Sequence
  Synthetic
  REPEAT (1)..(2): PR repeats
PRALIVQHNNCNNTWAFSAHVARSRSWEPNSPLMFNLFKSFPAFISHLQNDDNRI

SEQ ID NO: 42 - protein, 26 aa, Artificial Sequence
  Synthetic
GLDAGLCSSKAQFTPSLNIKILCTGV

SEQ ID NO: 43 - protein, 28 aa, Artificial Sequence
  Synthetic
  REPEAT (1)..(2): GR repeats
GRGLDAGLCSSKAQFTPSLNIKILCTGV

SEQ ID NO: 44 - protein, 32 aa, Artificial Sequence
  Synthetic
LTQVFAALKLSLHHLSTLKYCVLGFNNKTVQM

SEQ ID NO: 45 - protein, 33 aa, Artificial Sequence
  Synthetic
  REPEAT (1)..(1): G repeats
GLTQVFAALKLSLHHLSTLKYCVLGFNNKTVQM

SEQ ID NO: 46 - protein, 2 aa, Artificial Sequence
  Synthetic
  REPEAT (1)..(2): GA repeats
GA

SEQ ID NO: 47 - protein, 20 aa, Artificial Sequence
  Synthetic
AAAAAAAAAAAAAAAAAAAA

SEQ ID NO: 48 - protein, 18 aa, Artificial Sequence
  Synthetic
LLLLLLLLLLLLLLLLLL

SEQ ID NO: 49 - protein, 20 aa, Artificial Sequence
  Synthetic
SSSSSSSSSSSSSSSSSSSS

SEQ ID NO: 50 - protein, 20 aa, Artificial Sequence
  Synthetic
CCCCCCCCCCCCCCCCCCCC

SEQ ID NO: 51 - protein, 20 aa, Artificial Sequence
  Synthetic
GPGPGPGPGPGPGPGPGPGP

SEQ ID NO: 52 - protein, 20 aa, Artificial Sequence
  Synthetic
GAGAGAGAGAGAGAGAGAGA

SEQ ID NO: 53 - protein, 20 aa, Artificial Sequence
  Synthetic
GRGRGRGRGRGRGRGRGRGR

SEQ ID NO: 54 - protein, 20 aa, Artificial Sequence
  Synthetic
PAPAPAPAPAPAPAPAPAPA

SEQ ID NO: 55 - protein, 20 aa, Artificial Sequence
  Synthetic
PRPRPRPRPRPRPRPRPRPR

SEQ ID NO: 56 - protein, 12 aa, Artificial Sequence
  Synthetic
LPACLPACLPAC

SEQ ID NO: 57 - protein, 12 aa, Artificial Sequence
  Synthetic
QAGRQAGRQAGR

SEQ ID NO: 58 - DNA, 6 bp, Artificial Sequence
  Synthetic
cctcgt

SEQ ID NO: 59 - DNA, 6 bp, Artificial Sequence
  Synthetic
ccccgt

SEQ ID NO: 60 - DNA, 6 bp, Artificial Sequence
  Synthetic
ccacgt

SEQ ID NO: 61 - DNA, 6 bp, Artificial Sequence
  Synthetic
ccgcgt

SEQ ID NO: 62 - DNA, 6 bp, Artificial Sequence
  Synthetic
cctcgc

SEQ ID NO: 63 - DNA, 6 bp, Artificial Sequence
  Synthetic
ccccgc

SEQ ID NO: 64 - DNA, 6 bp, Artificial Sequence
  Synthetic
ccacgc

SEQ ID NO: 65 - DNA, 6 bp, Artificial Sequence
  Synthetic
ccgcgc

SEQ ID NO: 66 - DNA, 6 bp, Artificial Sequence
  Synthetic
cctcga

SEQ ID NO: 67 - DNA, 6 bp, Artificial Sequence
  Synthetic
ccccga

SEQ ID NO: 68 - DNA, 6 bp, Artificial Sequence
  Synthetic
ccacga

SEQ ID NO: 69 - DNA, 6 bp, Artificial Sequence
  Synthetic
ccgcga

SEQ ID NO: 70 - DNA, 6 bp, Artificial Sequence
  Synthetic
cctcgg

SEQ ID NO: 71 - DNA, 6 bp, Artificial Sequence
  Synthetic
ccccgg

SEQ ID NO: 72 - DNA, 6 bp, Artificial Sequence
  Synthetic
ccacgg

SEQ ID NO: 73 - DNA, 6 bp, Artificial Sequence
  Synthetic
ccgcgg

SEQ ID NO: 74 - DNA, 6 bp, Artificial Sequence
  Synthetic
cctaga

SEQ ID NO: 75 - DNA, 6 bp, Artificial Sequence
  Synthetic
cccaga

SEQ ID NO: 76 - DNA, 6 bp, Artificial Sequence
  Synthetic
ccaaga

SEQ ID NO: 77 - DNA, 6 bp, Artificial Sequence
  Synthetic
ccgaga

SEQ ID NO: 78 - DNA, 6 bp, Artificial Sequence
  Synthetic
cctagg

SEQ ID NO: 79 - DNA, 6 bp, Artificial Sequence
  Synthetic
cccagg

SEQ ID NO: 80 - DNA, 6 bp, Artificial Sequence
  Synthetic
ccaagg

SEQ ID NO: 81 - DNA, 6 bp, Artificial Sequence
  Synthetic
ccgagg

SEQ ID NO: 82 - DNA, 6 bp, Artificial Sequence
  Synthetic
ggtcgt

SEQ ID NO: 83 - DNA, 6 bp, Artificial Sequence
  Synthetic
ggccgt

SEQ ID NO: 84 - DNA, 6 bp, Artificial Sequence
  Synthetic
ggacgt

SEQ ID NO: 85 - DNA, 6 bp, Artificial Sequence
  Synthetic
gggcgt

SEQ ID NO: 86 - DNA, 6 bp, Artificial Sequence
  Synthetic
ggtcgc

SEQ ID NO: 87 - DNA, 6 bp, Artificial Sequence
  Synthetic
ggccgc

SEQ ID NO: 88 - DNA, 6 bp, Artificial Sequence
  Synthetic
ggacgc

SEQ ID NO: 89 - DNA, 6 bp, Artificial Sequence
  Synthetic
gggcgc

SEQ ID NO: 90 - DNA, 6 bp, Artificial Sequence
  Synthetic
ggtcga

SEQ ID NO: 91 - DNA, 6 bp, Artificial Sequence
  Synthetic
ggccga

SEQ ID NO: 92 - DNA, 6 bp, Artificial Sequence
  Synthetic
ggacga

SEQ ID NO: 93 - DNA, 6 bp, Artificial Sequence
  Synthetic
gggcga

SEQ ID NO: 94 - DNA, 6 bp, Artificial Sequence
  Synthetic
ggtcgg

SEQ ID NO: 95 - DNA, 6 bp, Artificial Sequence
  Synthetic
ggccgg

SEQ ID NO: 96 - DNA, 6 bp, Artificial Sequence
  Synthetic
ggacgg

SEQ ID NO: 97 - DNA, 6 bp, Artificial Sequence
  Synthetic
gggcgg

SEQ ID NO: 98 - DNA, 6 bp, Artificial Sequence
  Synthetic
ggtaga

SEQ ID NO: 99 - DNA, 6 bp, Artificial Sequence
  Synthetic
ggcaga

SEQ ID NO: 100 - DNA, 6 bp, Artificial Sequence
  Synthetic
ggaaga

SEQ ID NO: 101 - DNA, 6 bp, Artificial Sequence
  Synthetic
gggaga

SEQ ID NO: 102 - DNA, 6 bp, Artificial Sequence
  Synthetic
ggtagg

SEQ ID NO: 103 - DNA, 6 bp, Artificial Sequence
  Synthetic
ggcagg

SEQ ID NO: 104 - DNA, 6 bp, Artificial Sequence
  Synthetic
ggaagg

SEQ ID NO: 105 - DNA, 6 bp, Artificial Sequence
  Synthetic
gggagg

SEQ ID NO: 106 - protein, 10 aa, Artificial Sequence
  Synthetic
PRPRPRPRPR

SEQ ID NO: 107 - protein, 10 aa, Artificial Sequence
  Synthetic
GRGRGRGRGR

SEQ ID NO: 108 - protein, 9 aa, Artificial Sequence
  Synthetic
SSSSSSSSS

SEQ ID NO: 109 - protein, 120 aa, Artificial Sequence
  Synthetic
EVQLQESGGGSVQPGGSLKLSCAASGFAFSNYGMSWVRQTPDKRLELVTTINSDGDSTFYPDSVKGRFTISRDNAKNALYLQMSSLKSDDTAMYYCARVGGNYDFAMDYWGQGTSVIVSS

SEQ ID NO: 110 - protein, 113 aa, Artificial Sequence
  Synthetic
DIVMSQFPSSLAVSAGDKVTMSCKSSQSLLNSRTRKNYLAWYQQKPGQSPKLLIYWTSTRESGVPDRFTGSRSGTDFTLTISSVQAEDLAVYYCKQSYNNPWTFGGGTKLEIK

SEQ ID NO: 111 - protein, 120 aa, Artificial Sequence
  Synthetic
EVQLQESGGGSVQPGGALQLSCAASGFTFSSHGMSWVRQTPDKRLEMVATINSNGGSTYYPDSVKGRFIISRDNAKNTLYLQMSSLKSEDTAMYYCARVGDNDDFAMGYWGQGTSVTVSS

SEQ ID NO: 112 - protein, 113 aa, Artificial Sequence
  Synthetic
DIVMSQSPSSLAVSEGEKVTLTCKSSQSLFNSRTRKNYLAWYQQKPGQPPKLLIYWTSTRESGVPDRFTGSGYGTDFTLTISSVQAEDLAVYYCKQSYNNPWTFGGGTKLEIK

SEQ ID NO: 113 - protein, 121 aa, Artificial Sequence
  Synthetic
EVQLQESGSEVVRPGASVKLSCKASGYTFTSYWLHWVKQRPGQGLEWIGNVYPGSGLTGYDEKFRTKATVTVDTSSSTAYMQLSSLTTEDSAVYYCTRSAYSWYDYGMDCWGQGTSVTVST

SEQ ID NO: 114 - protein, 106 aa, Artificial Sequence
  Synthetic
QIVLTQSPEILSASPGEKVTMTCNATSSVNYMHWYQQKSGTSPKRWIYDTSKLASGVPARFSGSGSGTSYSLTISSMEAEDAAAYYCHQWSSNPPTFGSGTKLEIK

SEQ ID NO: 115 - protein, 121 aa, Artificial Sequence
  Synthetic
EVQLQQSGAELVRSGASVKLSCTASGFNIRDFYIQWVKQRPEQGLEWIGWIDPENGDTEYAPKFQGKATMTADTSSNTAYLQLSSLTSEDTAVYYCNAGDYDSHYYSMDYWGQGTSVTVSS

SEQ ID NO: 116 - protein, 112 aa, Artificial Sequence
  Synthetic
DVLMTQTPLSLPVSLGDQASISCRSSRSIVHSNGNTYLEWYLQKPGQSPKLLIYKVSNRFSGVPDRFSGSGSGTDFTLKISRVEADDLGVYYCYQVSHVPWTFGGGTKLEIK

SEQ ID NO: 117 - protein, 8 aa, Artificial Sequence
  Synthetic
GFAFSNYG

SEQ ID NO: 118 - protein, 12 aa, Artificial Sequence
  Synthetic
QSLLNSRTRKNY

SEQ ID NO: 119 - protein, 8 aa, Artificial Sequence
  Synthetic
GFTFSSHG

SEQ ID NO: 120 - protein, 12 aa, Artificial Sequence
  Synthetic
QSLFNSRTRKNY

SEQ ID NO: 121 - protein, 8 aa, Artificial Sequence
  Synthetic
GYTFTSYW

SEQ ID NO: 122 - protein, 5 aa, Artificial Sequence
  Synthetic
SSVNY

SEQ ID NO: 123 - protein, 5 aa, Artificial Sequence
  Synthetic
DFYIQ

SEQ ID NO: 124 - protein, 16 aa, Artificial Sequence
  Synthetic
RSSRSIVHSNGNTYLE

SEQ ID NO: 125 - protein, 8 aa, Artificial Sequence
  Synthetic
INSDGDST

SEQ ID NO: 126 - protein, 3 aa, Artificial Sequence
  Synthetic
WTS

SEQ ID NO: 127 - protein, 8 aa, Artificial Sequence
  Synthetic
INSNGGST

SEQ ID NO: 128 - protein, 12 aa, Artificial Sequence
  Synthetic
DRMPSVGEGAEG

SEQ ID NO: 129 - protein, 8 aa, Artificial Sequence
  Synthetic
VYPGSGLT

SEQ ID NO: 130 - protein, 3 aa, Artificial Sequence
  Synthetic
DTS

SEQ ID NO: 131 - protein, 17 aa, Artificial Sequence
  Synthetic
WIDPENGDTEYAPKFQG

SEQ ID NO: 132 - protein, 7 aa, Artificial Sequence
  Synthetic
KVSNRFS

SEQ ID NO: 133 - protein, 13 aa, Artificial Sequence
  Synthetic
ARVGGNYDFAMDY

SEQ ID NO: 134 - protein, 9 aa, Artificial Sequence
  Synthetic
KQSYNNPWT

SEQ ID NO: 135 - protein, 13 aa, Artificial Sequence
  Synthetic
ARVGDNDDFAMGY

SEQ ID NO: 136 - protein, 14 aa, Artificial Sequence
  Synthetic
  REPEAT (1)..(2): GR repeats
GRDRMPSVGEGAEG

SEQ ID NO: 137 - protein, 14 aa, Artificial Sequence
  Synthetic
TRSAYSWYDYGMDC

SEQ ID NO: 138 - protein, 9 aa, Artificial Sequence
  Synthetic
HQWSSNPPT

SEQ ID NO: 139 - protein, 12 aa, Artificial Sequence
  Synthetic
GDYDSHYYSMDY

SEQ ID NO: 140 - protein, 9 aa, Artificial Sequence
  Synthetic
YQVSHVPWT

SEQ ID NO: 141 - DNA, 15 bp, Artificial Sequence
  Synthetic
gacttctatattcag

SEQ ID NO: 142 - DNA, 48 bp, Artificial Sequence
  Synthetic
agatctagtcggagcattgtacatagtaatggaaacacctatttagaa

SEQ ID NO: 143 - DNA, 51 bp, Artificial Sequence
  Synthetic
tggattgatcctgagaatggtgatactgaatatgccccgaaattccagggc

SEQ ID NO: 144 - DNA, 21 bp, Artificial Sequence
  Synthetic
aaagtttccaaccgattttct

SEQ ID NO: 145 - DNA, 36 bp, Artificial Sequence
  Synthetic
ggggactatgattcccattactattctatggactac

SEQ ID NO: 146 - DNA, 27 bp, Artificial Sequence
  Synthetic
tatcaagtttcacatgttccgtggacg

SEQ ID NO: 147 - DNA, 361 bp, Artificial Sequence
  Synthetic
gaggtgcagctgcaggagtctgggggaggctcagtgcagcctggagggtccctgaaactctcctgcgcagcctctggattcgctttcagtaactatggcatgtcttgggttcgccagactccagacaagaggctggagttggtcacaaccattaatagtgatggtgatagtaccttttatccagacagtgtgaagggccgattcaccatctccagagacaatgccaagaacgccctgtacctgcaaatgagcagtctgaagtcagacgacacagccatgtattactgtgcaagagtgggaggtaactacgactttgctatggactactggggtcagggaacctcagtcatcgtctcctcag

SEQ ID NO: 148 - DNA, 340 bp, Artificial Sequence
  Synthetic
gacattgtgatgtcacagtttccatcctccctggctgtgtcagcaggagataaggtcactatgagctgcaaatccagtcagagtctgctcaacagtaggacccgaaagaactacttggcttggtaccagcagaaaccagggcagtctcctaaactactgatctactggacatccactcgggaatctggggtccctgatcgcttcacaggcagtcgatctgggacagatttcactctcaccatcagcagtgtgcaggctgaagacctggcagtttattactgcaagcaatcttataataatccgtggacgttcggtggaggcaccaagctggaaataaaac

SEQ ID NO: 149 - DNA, 361 bp, Artificial Sequence
  Synthetic
gaggtgcagctgcaggagtctgggggaggctcagtgcagcctggaggggccctgcaactctcctgtgcagcctctggattcactttcagtagtcatggcatgtcttgggttcgccagactccagacaagaggctggaaatggtcgcaaccattaatagtaatggtgggagtacctattacccagacagtgtgaagggccgattcatcatctccagagacaatgccaaaaacaccctgtacctgcaaatgagcagtctgaagtctgaggacacagccatgtattactgtgcaagagtgggagataacgacgactttgctatgggctactggggtcaaggaacctcagtcaccgtctcctcag

SEQ ID NO: 150 - DNA, 340 bp, Artificial Sequence
  Synthetic
gacattgtgatgtcacagtctccatcctccctggctgtgtcagaaggagagaaggtcactttaacctgcaaatccagtcagagtttgttcaacagtagaacccgaaagaactacttggcttggtaccagcagaaaccagggcagcctcctaaactgttgatctactggacatccactagggaatctggggtccctgatcgcttcacaggcagtggatatgggacagatttcactctcaccatcagcagtgtgcaggctgaagacctggcagtttattactgcaaacaatcttataataatccgtggacgttcggtggaggcaccaagttggaaataaaac

SEQ ID NO: 151 - DNA, 364 bp, Artificial Sequence
  Synthetic
gaggtgcagctgcaggagtctgggtctgaggtggtgaggcctggagcttcagtgaagctgtcctgcaaggcttctggctacacattcaccagctactggctgcactgggtgaagcagaggcctggacaaggccttgagtggattggaaatgtttatcctggtagtggtcttactggctacgatgaaaaattcaggaccaaggccacagtgactgtagacacatcctccagcacagcctacatgcaactcagcagcctgacaactgaggactctgcggtctattactgtacaagatcggcctactcttggtacgactatggaatggactgctggggtcaaggaacctcagtcacagtctctacag

SEQ ID NO: 152 - DNA, 319 bp, Artificial Sequence
  Synthetic
caaattgttctcacccagtctccagaaatcttgtctgcatctccaggggagaaggtcaccatgacctgcaatgccacctcaagtgtaaattatatgcactggtaccagcagaagtcaggcacctcccccaaaagatggatttatgacacatccaaactggcttctggagtccctgctcgcttcagtggcagtgggtctgggacctcttattctctcacaatcagcagcatggaggctgaagatgctgccgcttattactgccaccagtggagtagtaacccacccacgttcggctcggggacaaagctggaaatcaaac

SEQ ID NO: 153 - DNA, 363 bp, Artificial Sequence
  Synthetic
gaggttcagctgcagcagtctggggcagaacttgtgaggtcaggggcctcagtcaagttgtcctgcacagcttctggcttcaacattagagacttctatattcagtgggtgaaacagaggcctgaacagggcctggagtggattggatggattgatcctgagaatggtgatactgaatatgccccgaaattccagggcaaggccactatgactgcagacacatcctccaacacagcctacctgcagctcagcagcctgacatctgaggacactgccgtctattactgtaatgcaggggactatgattcccattactattctatggactactggggtcaaggaacctctgtcaccgtctcctca

SEQ ID NO: 154 - DNA, 336 bp, Artificial Sequence
  Synthetic
gatgttttgatgacccaaactccactctccctgcctgtcagtcttggagatcaagcctccatctcttgcagatctagtcggagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgatgatctgggagtttattactgctatcaagtttcacatgttccgtggacgttcggtggaggcaccaagctggaaatcaaa

SEQ ID NO: 155 - protein, 25 aa, Artificial Sequence
  Synthetic
EVQLQESGGGSVQPGGSLKLSCAAS

SEQ ID NO: 156 - protein, 26 aa, Artificial Sequence
  Synthetic
DIVMSQFPSSLAVSAGDKVTMSCKSS

SEQ ID NO: 157 - protein, 25 aa, Artificial Sequence
  Synthetic
EVQLQESGGGSVQPGGALQLSCAAS

SEQ ID NO: 158 - protein, 26 aa, Artificial Sequence
  Synthetic
DIVMSQSPSSLAVSEGEKVTLTCKSS

SEQ ID NO: 159 - protein, 25 aa, Artificial Sequence
  Synthetic
EVQLQESGSEVVRPGASVKLSCKAS

SEQ ID NO: 160 - protein, 26 aa, Artificial Sequence
  Synthetic
QIVLTQSPEILSASPGEKVTMTCNAT

SEQ ID NO: 161 - protein, 30 aa, Artificial Sequence
  Synthetic
EVQLQQSGAELVRSGASVKLSCTASGFNIR

SEQ ID NO: 162 - protein, 23 aa, Artificial Sequence
  Synthetic
DVLMTQTPLSLPVSLGDQASISC

SEQ ID NO: 163 - protein, 17 aa, Artificial Sequence
  Synthetic
MSWVRQTPDKRLELVTT

SEQ ID NO: 164 - protein, 17 aa, Artificial Sequence
  Synthetic
LAWYQQKPGQSPKLLIY

SEQ ID NO: 165 - protein, 17 aa, Artificial Sequence
  Synthetic
MSWVRQTPDKRLEMVAT

SEQ ID NO: 166 - protein, 17 aa, Artificial Sequence
  Synthetic
LAWYQQKPGQPPKLLIY

SEQ ID NO: 167 - protein, 17 aa, Artificial Sequence
  Synthetic
LHWVKQRPGQGLEWIGN

SEQ ID NO: 168 - protein, 17 aa, Artificial Sequence
  Synthetic
MHWYQQKSGTSPKRWIY

SEQ ID NO: 169 - protein, 14 aa, Artificial Sequence
  Synthetic
WVKQRPEQGLEWIG

SEQ ID NO: 170 - protein, 15 aa, Artificial Sequence
  Synthetic
WYLQKPGQSPKLLIY

SEQ ID NO: 171 - protein, 38 aa, Artificial Sequence
  Synthetic
FYPDSVKGRFTISRDNAKNALYLQMSSLKSDDTAMYYC

SEQ ID NO: 172 - protein, 36 aa, Artificial Sequence
  Synthetic
TRESGVPDRFTGSRSGTDFTLTISSVQAEDLAVYYC

SEQ ID NO: 173 - protein, 38 aa, Artificial Sequence
  Synthetic
YYPDSVKGRFIISRDNAKNTLYLQMSSLKSEDTAMYYC

SEQ ID NO: 174 - protein, 36 aa, Artificial Sequence
  Synthetic
TRESGVPDRFTGSGYGTDFTLTISSVQAEDLAVYYC

SEQ ID NO: 175 - protein, 38 aa, Artificial Sequence
  Synthetic
GYDEKFRTKATVTVDTSSSTAYMQLSSLTTEDSAVYYC

SEQ ID NO: 176 - protein, 36 aa, Artificial Sequence
  Synthetic
KLASGVPARFSGSGSGTSYSLTISSMEAEDAAAYYC

SEQ ID NO: 177 - protein, 32 aa, Artificial Sequence
  Synthetic
KATMTADTSSNTAYLQLSSLTSEDTAVYYCNA

SEQ ID NO: 178 - protein, 32 aa, Artificial Sequence
  Synthetic
GVPDRFSGSGSGTDFTLKISRVEADDLGVYYC

SEQ ID NO: 179 - protein, 11 aa, Artificial Sequence
  Synthetic
WGQGTSVIVSS

SEQ ID NO: 180 - protein, 10 aa, Artificial Sequence
  Synthetic
FGGGTKLEIK

SEQ ID NO: 181 - protein, 11 aa, Artificial Sequence
  Synthetic
WGQGTSVTVSS

SEQ ID NO: 182 - protein, 33 aa, Artificial Sequence
  Synthetic
GTGCLQWVKVQKGRSEEVGMEEGEEGGGEELRK

SEQ ID NO: 183 - protein, 11 aa, Artificial Sequence
  Synthetic
WGQGTSVTVST

SEQ ID NO: 184 - protein, 10 aa, Artificial Sequence
  Synthetic
FGSGTKLEIK

SEQ ID NO: 185 - protein, 35 aa, Artificial Sequence
  Synthetic
  REPEAT (1)..(2): GA repeats
GAGTGCLQWVKVQKGRSEEVGMEEGEEGGGEELRK

SEQ ID NO: 186 - protein, 5 aa, Artificial Sequence
  Synthetic
DAFSG

SEQ ID NO: 187 - DNA, 90 bp, Artificial Sequence
  Synthetic
gaggttcagctgcagcagtctggggcagaacttgtgaggtcaggggcctcagtcaagttgtcctgcacagcttctggcttcaacattaga

SEQ ID NO: 188 - DNA, 69 bp, Artificial Sequence
  Synthetic
gatgttttgatgacccaaactccactctccctgcctgtcagtcttggagatcaagcctccatctcttgc

SEQ ID NO: 189 - DNA, 42 bp, Artificial Sequence
  Synthetic
tgggtgaaacagaggcctgaacagggcctggagtggattgga

SEQ ID NO: 190 - DNA, 45 bp, Artificial Sequence
  Synthetic
tggtacctgcagaaaccaggccagtctccaaagctcctgatctac

SEQ ID NO: 191 - DNA, 96 bp, Artificial Sequence
  Synthetic
aaggccactatgactgcagacacatcctccaacacagcctacctgcagctcagcagcctgacatctgaggacactgccgtctattactgtaatgca

SEQ ID NO: 192 - DNA, 96 bp, Artificial Sequence
  Synthetic
ggggtcccagacaggttcagtggcagtggatcagggacagatttcacactcaagatcagcagagtggaggctgatgatctgggagtttattactgc

SEQ ID NO: 193 - DNA, 33 bp, Artificial Sequence
  Synthetic
tggggtcaaggaacctctgtcaccgtctcctca

SEQ ID NO: 194 - DNA, 30 bp, Artificial Sequence
  Synthetic
ttcggtggaggcaccaagctggaaatcaaa

SEQ ID NO: 195 - protein, 330 aa, Artificial Sequence
  Synthetic
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 196 - protein, 107 aa, Artificial Sequence
  Synthetic
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 197 - DNA, 990 bp, Artificial Sequence
  Synthetic
gccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 198 - DNA, 321 bp, Artificial Sequence
  Synthetic
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 199 - DNA, 60 bp, Artificial Sequence
  Synthetic
gagagagagagagagagagagagagagagagagagagagagagagagagagagagagaga

SEQ ID NO: 200 - protein, 7 aa, Artificial Sequence
  Synthetic
  REPEAT (1)..(2): GQ repeats
GQDAFSG

SEQ ID NO: 201 - protein, 13 aa, Artificial Sequence
  Synthetic
  site (10)..(11): modified by dPEG4
SSSSSSSSSSCKK

SEQ ID NO: 202 - protein, 17 aa, Artificial Sequence
  Synthetic
RPRPRPRPRPRPRPRPC

SEQ ID NO: 203 - protein, 54 aa, Artificial Sequence
  Synthetic
PCSSPVHLIPDLFVVEFREWSEMDRVGKKGEREEGSLFFQLWALSCNVQSEEKI

SEQ ID NO: 204 - protein, 56 aa, Artificial Sequence
  Synthetic
  REPEAT (1)..(2): LP repeats
LPPCSSPVHLIPDLFVVEFREWSEMDRVGKKGEREEGSLFFQLWALSCNVQSEEKI

SEQ ID NO: 205 - protein, 9 aa, Artificial Sequence
  Synthetic
LLLPCPSDS

SEQ ID NO: 206 - protein, 11 aa, Artificial Sequence
  Synthetic
  REPEAT (1)..(2): SP repeats
SPLLLPCPSDS

SEQ ID NO: 207 - protein, 7 aa, Artificial Sequence
  Synthetic
PAPPLSI

SEQ ID NO: 208 - protein, 9 aa, Artificial Sequence
  Synthetic
  REPEAT (1)..(2): SL repeats
SLPAPPLSI

SEQ ID NO: 209 - protein, 41 aa, Artificial Sequence
  Synthetic
GDFKQEKRKLLLLREGSRIETFGIQKLIQTFSSTCLFASTE

SEQ ID NO: 210 - protein, 43 aa, Artificial Sequence
  Synthetic
  REPEAT (1)..(2): GE repeats
GEGDFKQEKRKLLLLREGSRIETFGIQKLIQTFSSTCLFASTE

SEQ ID NO: 211 - protein, 41 aa, Artificial Sequence
  Synthetic
WARGSLSSSRSPLTSLPWGLPQTQVSPRHTLHLCGASPDGP

SEQ ID NO: 212 - protein, 11 aa, Artificial Sequence
  Synthetic
TLSRKKENYYC

SEQ ID NO: 213 - protein, 13 aa, Artificial Sequence
  Synthetic
  REPEAT (1)..(2): RE repeats
RETLSRKKENYYC

SEQ ID NO: 214 - protein, 47 aa, Artificial Sequence
  Synthetic
DSTSRKALGPLLSLAPSCQAPSIPKPCHNPMLQEVFLQPTPAHLPPS

SEQ ID NO: 215 - protein, 49 aa, Artificial Sequence
  Synthetic
  REPEAT (1)..(2): GA repeats
GADSTSRKALGPLLSLAPSCQAPSIPKPCHNPMLQEVFLQPTPAHLPPS

SEQ ID NO: 216 - protein, 79 aa, Artificial Sequence
  Synthetic
IPPPEKPLGPSSALPPPAKPPVSPSPATIPCSRRSSSSPPQPTSHPPDSDVLSPAKPCDLEEVIFLLCKWGMRTTCLTG

SEQ ID NO: 217 - protein, 81 aa, Artificial Sequence
  Synthetic
  REPEAT (1)..(2): GQ repeats
GQIPPPEKPLGPSSALPPPAKPPVSPSPATIPCSRRSSSSPPQPTSHPPDSDVLSPAKPCDLEEVIFLLCKWGMRTTCLTG

SEQ ID NO: 218 - protein, 71 aa, Artificial Sequence
  Synthetic
FHLQKSPWAPPQPCPLLPSPQYPQALPQSHAPGGLPPAHPSPPPTLLTQMFCLLLSRVTWRKSFFSSVNGG

SEQ ID NO: 219 - protein, 73 aa, Artificial Sequence
  Synthetic
  REPEAT (1)..(2): GR repeats
GRFHLQKSPWAPPQPCPLLPSPQYPQALPQSHAPGGLPPAHPSPPPTLLTQMFCLLLSRVTWRKSFFSSVNGG

SEQ ID NO: 220 - protein, 83 aa, Artificial Sequence
  Synthetic
PSPSLPSPHAVSKWIPDTKTPTGVVPEVRPVNLGPRALPVPLKARVWVCSGAELKAAKLTGKVSPVIRDVQGYGWGRGDSHLW

SEQ ID NO: 221 - protein, 85 aa, Artificial Sequence
  Synthetic
  REPEAT (1)..(2): CP repeats
CPPSPSLPSPHAVSKWIPDTKTPTGVVPEVRPVNLGPRALPVPLKARVWVCSGAELKAAKLTGKVSPVIRDVQGYGWGRGDSHLW

SEQ ID NO: 222 - protein, 21 aa, Artificial Sequence
  Synthetic
RLFPLPMLSPNGSLTPRLQLG

SEQ ID NO: 223 - protein, 23 aa, Artificial Sequence
  Synthetic
  REPEAT (1)..(2): PA repeats
PARLFPLPMLSPNGSLTPRLQLG

SEQ ID NO: 224 - protein, 15 aa, Artificial Sequence
  Synthetic
QPVSSLSPCCLQMDP

SEQ ID NO: 225 - protein, 17 aa, Artificial Sequence
  Synthetic
  REPEAT (1)..(2): LP repeats
LPQPVSSLSPCCLQMDP

SEQ ID NO: 226 - protein, 54 aa, Artificial Sequence
  Synthetic
PSPSPSPSPRLPLPLMPSQSWTVLLPSRLTATSLPDSPASACRVPAIAGARRHA

SEQ ID NO: 227 - protein, 56 aa, Artificial Sequence
  Synthetic
  REPEAT (1)..(2): LS repeats
LSPSPSPSPSPRLPLPLMPSQSWTVLLPSRLTATSLPDSPASACRVPAIAGARRHA

SEQ ID NO: 228 - protein, 35 aa, Artificial Sequence
  Synthetic
PVSLSLSLSPSPSPSHAEPKLDGTAAISAHCNLPA

SEQ ID NO: 229 - protein, 37 aa, Artificial Sequence
  Synthetic
  REPEAT (1)..(2): LP repeats
LPPVSLSLSLSPSPSPSHAEPKLDGTAAISAHCNLPA

SEQ ID NO: 230 - protein, 78 aa, Artificial Sequence
  Synthetic
PRLPLPLPLPVSLSLSCRAKAGRYCCHLGSLQPPCLILLPQPAECLRLQARAATPDWFSFFFWWRWGFAVLAGLVSSS

SEQ ID NO: 231 - protein, 80 aa, Artificial Sequence
  Synthetic
  REPEAT (1)..(2): PS repeats
PSPRLPLPLPLPVSLSLSCRAKAGRYCCHLGSLQPPCLILLPQPAECLRLQARAATPDWFSFFFWWRWGFAVLAGLVSSS

SEQ ID NO: 232 - protein, 19 aa, Artificial Sequence
  Synthetic
GVKFLSINVMPTVLSSCGL

SEQ ID NO: 233 - protein, 21 aa, Artificial Sequence
  Synthetic
  REPEAT (1)..(2): GR repeats
GRGVKFLSINVMPTVLSSCGL

SEQ ID NO: 234 - protein, 26 aa, Artificial Sequence
  Synthetic
RGQILIYQCYAHCALQLWSVNYCGIT

SEQ ID NO: 235 - protein, 28 aa, Artificial Sequence
  Synthetic
  REPEAT (1)..(2): RE repeats
RERGQILIYQCYAHCALQLWSVNYCGIT

SEQ ID NO: 236 - protein, 40 aa, Artificial Sequence
  Synthetic
GSNSYLSMLCPLCSPAVVCELLWYNVTVQISLFRGFDHDL

SEQ ID NO: 237 - protein, 51 aa, Artificial Sequence
  Synthetic
PLPLPLPFPLPRSLPLPLPSPPLFDRVSLVTQSGVHWHNLGSLQPPPPRFR

SEQ ID NO: 238 - protein, 52 aa, Artificial Sequence
  Synthetic
  REPEAT (1)..(2): FP repeats
FPLPLPLPFPLPRSLPLPLPSPPLFDRVSLVTQSGVHWHNLGSLQPPPPRFR

SEQ ID NO: 239 - protein, 66 aa, Artificial Sequence
  Synthetic
FLSPSPFLSPSPFPFPFPFPFPFPVPFPFPSPPLPFLTESHWSPSLECTGTILAHCNLRLPGSGDC

SEQ ID NO: 240 - protein, 68 aa, Artificial Sequence
  Synthetic
  REPEAT (1)..(2): SP repeats
SPFLSPSPFLSPSPFPFPFPFPFPFPVPFPFPSPPLPFLTESHWSPSLECTGTILAHCNLRLPGSGDC

SEQ ID NO: 241 - protein, 43 aa, Artificial Sequence
  Synthetic
CPFPLPLSFPLPLSFPLPLSPSPSPSLSPSPFPSPSPPLPSPF

SEQ ID NO: 242 - protein, 45 aa, Artificial Sequence
  Synthetic
  REPEAT (1)..(2): LP repeats
LPCPFPLPLSFPLPLSFPLPLSPSPSPSLSPSPFPSPSPPLPSPF

SEQ ID NO: 243 - protein, 47 aa, Artificial Sequence
  Synthetic
GDREGKKVSSAEYISRLRSHHSKHYCSSDMLKQNSQTLLSLVTSKSK

SEQ ID NO: 244 - protein, 49 aa, Artificial Sequence
  Synthetic
  REPEAT (1)..(2): GE repeats
GEGDREGKKVSSAEYISRLRSHHSKHYCSSDMLKQNSQTLLSLVTSKSK

SEQ ID NO: 245 - protein, 15 aa, Artificial Sequence
  Synthetic
TGRERKFQALNIFQD

SEQ ID NO: 246 - protein, 17 aa, Artificial Sequence
  Synthetic
  REPEAT (1)..(2): GK repeats
GKTGRERKFQALNIFQD

SEQ ID NO: 247 - protein, 10 aa, Artificial Sequence
  Synthetic
GQGGKESFKR

SEQ ID NO: 248 - protein, 12 aa, Artificial Sequence
  Synthetic
  REPEAT (1)..(2): GR repeats
GRGQGGKESFKR

SEQ ID NO: 249 - protein, 60 aa, Artificial Sequence
  Synthetic
PLPPGAYSPLPPGVYSPLLPGVYSLCPGVYSPASWPSTFCRSCCFHTFCPMGDGLCSVGP

SEQ ID NO: 250 - protein, 68 aa, Artificial Sequence
  Synthetic
  REPEAT (1)..(8): FTPLSLPV repeats
FTPLSLPVPLPPGAYSPLPPGVYSPLLPGVYSLCPGVYSPASWPSTFCRSCCFHTFCPMGDGLCSVGP

SEQ ID NO: 251 - protein, 141 aa, Artificial Sequence
  Synthetic
AGLAVFTRSAPWVMDCVLWGPEITQATEQTFSPQEVLAASSSLPASVPALCPQPPSPTAPAASPRTLGKCIPSLGPGTGPVSHVAALDPPSPVLVPHAGQASGAPVCGPPQLVAQHQACNQLLVNIGPVAFSDTNKSEGSW

SEQ ID NO: 252 - protein, 149 aa, Artificial Sequence
  Synthetic
  REPEAT (1)..(8): LLPSPSRC repeats
LLPSPSRCAGLAVFTRSAPWVMDCVLWGPEITQATEQTFSPQEVLAASSSLPASVPALCPQPPSPTAPAASPRTLGKCIPSLGPGTGPVSHVAALDPPSPVLVPHAGQASGAPVCGPPQLVAQHQACNQLLVNIGPVAFSDTNKSEGSW

SEQ ID NO: 253 - protein, 79 aa, Artificial Sequence
  Synthetic
CLLPSPSRCLLPSASRCLLPSPSRCLLPSSSRCLLPSASRCLLPSASRCLLPVSWCLLPCFLAIYLLPVLLFSHVLPHG

SEQ ID NO: 254 - protein, 87 aa, Artificial Sequence
  Synthetic
  REPEAT (1)..(8): YSPLPPGV repeats
YSPLPPGVCLLPSPSRCLLPSASRCLLPSPSRCLLPSSSRCLLPSASRCLLPSASRCLLPVSWCLLPCFLAIYLLPVLLFSHVLPHG

SEQ ID NO: 255 - protein, 8 aa, Artificial Sequence
  Synthetic
  REPEAT (1)..(8): HREGEGSK repeats
HREGEGSK

SEQ ID NO: 256 - protein, 74 aa, Artificial Sequence
  Synthetic
GKHRRRKCRHVRSAPTPMGQRWGCSAPASQLVGVLQQANHSPSERLGTLPPHLGHGWMQKESRFATVLHSHLCW

SEQ ID NO: 257 - protein, 82 aa, Artificial Sequence
  Synthetic
  REPEAT (1)..(8): TGRERGVN repeats
TGRERGVNGKHRRRKCRHVRSAPTPMGQRWGCSAPASQLVGVLQQANHSPSERLGTLPPHLGHGWMQKESRFATVLHSHLCW

SEQ ID NO: 258 - protein, 6 aa, Artificial Sequence
  Synthetic
  REPEAT (1)..(6): PGGRGE repeats
PGGRGE

SEQ ID NO: 259 - protein, 42 aa, Artificial Sequence
  Synthetic
  REPEAT (1)..(2): GE repeats
GEGSNSYLSMLCPLCSPAVVCELLWYNVTVQISLFRGFDHDL

SEQ ID NO: 260 - protein, 4 aa, Artificial Sequence
  Synthetic
  REPEAT (1)..(4): LPAC repeats
LPAC

SEQ ID NO: 261 - protein, 4 aa, Artificial Sequence
  Synthetic
  REPEAT (1)..(4): QAGR repeats
QAGR

SEQ ID NO: 262 - protein, 8 aa, Artificial Sequence
  Synthetic
  REPEAT (1)..(8): FTPLSLPV repeats
FTPLSLPV

SEQ ID NO: 263 - protein, 8 aa, Artificial Sequence
  Synthetic
  REPEAT (1)..(8): LLPSPSRC repeats
LLPSPSRC

SEQ ID NO: 264 - protein, 8 aa, Artificial Sequence
  Synthetic
  REPEAT (1)..(8): YSPLPPGV repeats
YSPLPPGV

SEQ ID NO: 265 - protein, 8 aa, Artificial Sequence
  Synthetic
  REPEAT (1)..(8): TGRERGVN repeats
TGRERGVN

SEQ ID NO: 266 - protein, 8 aa, Artificial Sequence
  Synthetic
  REPEAT (1)..(8): GRQRGVNT repeats
GRQRGVNT

SEQ ID NO: 267 - protein, 8 aa, Artificial Sequence
  Synthetic
  REPEAT (1)..(8): GSKHREAE repeats
GSKHREAE

SEQ ID NO: 268 - protein, 8 aa, Artificial Sequence
  Synthetic
FTPLSLPV

SEQ ID NO: 269 - protein, 8 aa, Artificial Sequence
  Synthetic
LLPSPSRC

SEQ ID NO: 270 - protein, 8 aa, Artificial Sequence
  Synthetic
YSPLPPGV

SEQ ID NO: 271 - protein, 8 aa, Artificial Sequence
  Synthetic
HREGEGSK

SEQ ID NO: 272 - protein, 8 aa, Artificial Sequence
  Synthetic
TGRERGVN

SEQ ID NO: 273 - protein, 6 aa, Artificial Sequence
  Synthetic
PGGRGE

SEQ ID NO: 274 - protein, 43 aa, Artificial Sequence
  Synthetic
  REPEAT (1)..(2): PA repeats
PAWARGSLSSSRSPLTSLPWGLPQTQVSPRHTLHLCGASPDGP

SEQ ID NO: 275 - protein, 12 aa, Artificial Sequence
  Synthetic
GHVAHSLPPGLL

SEQ ID NO: 276 - protein, 14 aa, Artificial Sequence
  Synthetic
  REPEAT (1)..(2): LP repeats
LPGHVAHSLPPGLL

SEQ ID NO: 277 - protein, 59 aa, Artificial Sequence
  Synthetic
CLGTWLTLFLQVSFNITSLGTPTDSGLPATHTSPLWCFSRWSLTSHVSNICPSCWAGYS

SEQ ID NO: 278 - protein, 61 aa, Artificial Sequence
  Synthetic
  REPEAT (1)..(2): CP repeats
CPCLGTWLTLFLQVSFNITSLGTPTDSGLPATHTSPLWCFSRWSLTSHVSNICPSCWAGYS

(19) United States
(12) Patent Application Publication
(10) Pub. No.: US 2022/0267776 A1
(43) Pub. Date: Aug. 25, 2022

(54) METHODS FOR TREATING RAN PROTEIN-ASSOCIATED NEUROLOGICAL DISEASES

(71) Applicant: University of Florida Research Foundation, Incorporated, Gainesville, FL (US)

(72) Inventors: Laura Ranum, Gainesville, FL (US); Lien Nguyen, Gainesville, FL (US)

(73) Assignee: University of Florida Research Foundation, Incorporated, Gainesville, FL (US)

(21) Appl. No.: 17/624,700

(22) PCT Filed: Jul. 2, 2020
(86) PCT No.: PCT/US20/40725

§ 371 Date: Jan. 4, 2022

Related U.S. Application Data
(60) Provisional application No. 62/871,031, filed on Jul. 5, 2019.
(60) Provisional application No. 63/025,096, filed on May 14, 2020.

(51) Int. Cl. C12N 15/113; C12N 15/115; C07K 16/40; A61K 38/45; A61K 31/445; A61K 31/55; A61K 31/13; A61K 31/27; A61P 25/28; C12Q 1/6883; G01N 33/68

(52) U.S. Cl. CPC C12N 15/1137; G01N 2333/914; C07K 16/40; A61K 38/45; A61K 31/445; A61K 31/55; A61K 31/13; A61K 31/27; A61P 25/28; C12Q 1/6883; G01N 33/6896; C12N 2310/11; C12N 2310/141; C12N 2310/531; C12N 2310/14; C12Q 2600/158; G01N 2800/2821; C12N 15/115

(57) ABSTRACT

Aspects of the disclosure relate to compositions and methods for the diagnosis and/or treatment of certain neurodegenerative diseases, for example those diseases associated with repeat-associated non-ATG (RAN) translation proteins, such as Alzheimer's disease (AD). In some embodiments, the disclosure relates to identifying a subject having a RAN protein-associated disease by detecting expression or activity of repeat-associated non-ATG (RAN) translation proteins (e.g., RAN proteins). In some embodiments, the disclosure relates to methods of treating a RAN protein-associated disease by administering to a subject in need thereof an agent that reduces expression or activity of RAN proteins.

### RELATED APPLICATIONS

This application is a national stage filing under 35 U.S.C. § 371 of International Patent Application Serial No. PCT/US2020/040725, filed Jul. 2, 2020, which claims the benefit under 35 U.S.C. § 119(e) of U.S. Provisional Application Ser. No. 62/871,031, filed Jul. 5, 2019, entitled “METHODS FOR TREATING ALZHEIMER'S DISEASE”, and U.S. Provisional Application Ser. No. 63/025,096, filed May 14, 2020, entitled “METHODS FOR TREATING RAN PROTEIN-ASSOCIATED NEUROLOGICAL DISEASES”, the entire contents of each of which are incorporated herein by reference.

### SEQUENCE LISTING

In accordance with 37 C.F.R. 1.52(e)(5), the present specification makes reference to a Sequence Listing (submitted electronically as a .txt file named “U120270068US02-SEQ”). The .txt file was generated on Dec. 8, 2021, and is 102,196 bytes in size. The Sequence Listing is herein incorporated by reference in its entirety.

### BACKGROUND

Microsatellite repeat expansions are known to cause more than forty neurodegenerative disorders. Molecular features common to many of these disorders include the accumulation of RNA foci containing sense and antisense expansion transcripts and the accumulation of proteins from repeat-associated non-AUG (RAN) translation. RAN translation can occur across a broad range of repeat lengths from pre-mutation lengths (˜30-40 repeats) to full expansions (up to 10,000 repeats). While repetitive elements account for a large portion of the human genome, the detection of repeats and repeat expansion mutations is challenging.

### SUMMARY

Described herein are compositions and methods for the diagnosis and treatment of certain neurological diseases associated with repeat associated non-ATG (RAN) proteins, including, for example, polySerine [polySer], poly(Proline-Arginine) [poly(PR)], and poly(Glycine-Arginine) [poly(GR)], etc. Mutations of certain repeat expansions (e.g., CAGG, CCTG, GGGGCC, GGCCCC, GGGGCA, CAG, and CTG) are associated with a number of different neurological diseases (e.g., amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE).

In a growing number of these diseases including, but not limited to, ALS or FTD, FXTAS, HD, SCA8, DM1 and DM2, expansion mutations have been shown to undergo a novel type of protein translation that occurs in multiple reading frames and does not require a canonical AUG initiation codon. This type of translation is called repeat associated non-ATG (RAN) translation and the proteins that are produced are called RAN proteins. There is growing evidence that RAN proteins are toxic and contribute to a growing number of diseases. It therefore is important to develop therapeutic strategies that reduce the level of RAN proteins to treat neurological diseases caused by repeat expansion mutations.

In some embodiments, compositions and methods for the diagnosis and treatment of certain neurological diseases associated with RAN proteins are disclosed. In some embodiments, the neurological disease associated with RAN proteins is selected from the group consisting of: amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE). In a specific embodiment, the neurological disease associated with RAN proteins is Alzheimer's Disease (AD).

Aspects of the disclosure relate to methods and compositions for the diagnosis and treatment of certain RAN protein-associated diseases, for example Alzheimer's disease and other neurological diseases or disorders. The disclosure is based, in part, on the discovery that certain RAN proteins, including for example, polySerine [polySer], poly(Proline-Arginine) [poly(PR)], and poly(Glycine-Arginine) [poly(GR)], accumulate in the brains of certain subjects having such diseases and that these RAN proteins can be detected in a biological sample (e.g., blood, serum, or cerebrospinal fluid (CSF) of a subject having or at risk of developing AD. Additional non-limiting examples of RAN proteins that accumulate in the brains of certain subjects having AD that can be detected in a biological sample of a subject having or at risk of developing AD include poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and/or poly(GSKHREAE) (SEQ ID NO: 267).

In some embodiments, an Enzyme-linked Immunosorbent assay (ELISA), ElectroChemiLuminescence Immuno assay (Meso Scale Discovery, MSD), digital ELISA technology (Single molecule array, SIMOA), and/or Dot blot assay is used to detect RAN proteins. In some embodiments, a rolling circle amplification-based ELISA (RCA-based ELISA) is use to detect RAN proteins. In some embodiments, a real-time PCR based ELISA (rtPCR-based ELISA) is used to detect RAN proteins. In some embodiments, the assay comprises antibodies against repeat motifs of RAN proteins described herein. In some embodiments, the assay comprises antibodies against C-terminal specific sequences of RAN proteins. In some embodiments, an assay is used for detecting expansion mutations. In some embodiments, the assay for detecting expansion mutations is repeat prime PCR, long-range PCR, and/or Southern blot. These assays use primers that bind to DNA sequences within and upstream, and/or downstream of the repeat or flanking the repeat.

In some embodiments, the RAN proteins present in subjects having or at risk of developing a neurological disease associated with RAN proteins are not transcribed from a C9orf72 genetic locus in the subject. In some embodiments, accumulations of repeat containing sense or antisense RNA containing repeat expansions may be detected using fluorescence in situ hybridization probes to detect the accumulating RNA. In some embodiments, the RNA accumulations present in subjects having or at risk of developing a neurological disease associated with RAN proteins are not transcribed from a C9orf72 genetic locus in the subject. In some embodiments, the genes producing the novel RAN proteins include open reading frame 80 of chromosome 2 (C2orf80), LRP8, CASP8, CRNDE, EXOC6B, SV2B, PPML1, ADARB2, GREB1, and/or MSMO1. In some embodiments, an assay is used to determine whether a RAN protein was expressed from one or more of C2orf80, LRP8, CASP8, CRNDE, EXOC6B, SV2B, PPML1, ADARB2, GREB1, and MSMO1. In some embodiments, the assay for identifying RNA foci is fluorescence in situ hybridization, probes=fluorophore (e.g., Cy3, Cy5, A555, A549, A488)-labeled DNA sequences that complement to repeat sequences at expanded loci. In some embodiments, the assay for identifying RNA foci is deactivated Cas9-based hybridization: probes=DNA sequences that complement to repeat sequences at expanded loci and fluorophore-labeled deactivated Cas9.

In some aspects, the disclosure provides a method for treating a RAN protein-associated neurological disease by administering to a subject diagnosed as having, or being at risk for, a RAN protein-associated neurological disease a therapeutic agent (e.g., one or more antisense oligonucleotides, anti-RAN antibodies, other therapeutic agents, or a combination of two or more thereof) for the treatment of the RAN protein-associated neurological disease, wherein the subject has been characterized as having a RAN protein-associated neurological disease by the detection of at least one RAN protein and/or at least one RNA encoding a RAN protein in a biological sample obtained from the subject. A subject may have a multiple of RAN proteins expressed from an expansion mutation. In some embodiments, antibodies targeting RAN proteins disclosed herein can be targeting one or multiple RAN proteins. In some embodiments, an individual antibody may target one or more RAN proteins. In some embodiments, a combination of two or more different antibodies may be used, wherein each antibody targets a different RAN protein. In some embodiments, RAN proteins are expressed in all three reading frames, from both sense and antisense transcripts.

In some embodiments, a RAN protein is poly(GR), poly(PR), and/or polySer. In some embodiments, a RAN protein is poly(CP), poly(GP), poly(G), poly(A), poly(GA), poly(GD), poly(GE), poly(GQ), poly(GT), poly(L), poly(LP), poly(LPAC) (SEQ ID NO: 260), poly(LS), poly(P), poly(PA), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and/or poly(GSKHREAE) (SEQ ID NO: 267). In some embodiments, a RAN protein is not transcribed from a C9orf72 locus of a subject.

In some embodiments, at least one RAN protein is encoded by a gene comprising between 2 and 10,000 repeats of a sequence selected from Table 1, Table 2, or Table 3.

In some embodiments, a therapeutic agent is a small molecule, interfering nucleic acid, modified interfering nucleic acid, DNA aptamer, RNA aptamer, peptide, protein, antibody, antibody drug conjugate, other large molecule, gene therapy (including a gene therapy designed to deliver one or more of the other enumerated types of therapeutic agents), a natural product, or an herbal medicine.

In some embodiments, a small molecule is a modifier of eukaryotic initiation factor 2 (eIF2), eukaryotic initiation factor 3 (eIF3), protein kinase R (PKR), p62 (sequestome-1 or ubiquitin binding protein), LC3 (microtubule associated protein 1 light chain 3) I subunit, LC3 II subunit, or Toll-like receptor 3 (TLR3).

In some embodiments, a small molecule is metformin or a pharmaceutically acceptable salt, co-crystal, tautomer, stereoisomer, solvate, hydrate, polymorph, isotopically enriched derivative, or prodrug thereof. In some embodiments, a small molecule is buformin, phenformin, metformin, or a derivative or functional analogue thereof. In some embodiments, a small molecule is an inhibitor of PKR such as TARBP2.

In some embodiments, an interfering nucleic acid is a dsRNA, siRNA, shRNA, miRNA, artificial miRNA (amiRNA), or antisense oligonucleotide (ASO). In some embodiments, an interfering nucleic acid modifies expression of eukaryotic initiation factor 2 (eIF2), eukaryotic initiation factor 3 (eIF3), protein kinase R (PKR), p62, LC3 I subunit, LC3 II subunit, or Toll-like receptor 3 (TLR3).

In some embodiments, an interfering nucleic acid modifies expression of eIF2A or eIF2a. In some embodiments, an interfering nucleic acid inhibits expression of one or more eIF3 subunits selected from the group consisting of eIF3a, eIF3b, eIF3c, eIF3d, eIF3e, eIF3f, eIF3g, eIF3h, eIF3i, eIF3j, eIF3k, eIF31, and eIF3m. In some embodiments, an interfering nucleic acid inhibits expression of protein kinase R (PKR). In some embodiments, an interfering nucleic acid inhibits expression of a gene comprising a nucleic acid repeat set forth in any one of Tables 1, 2, and 3. In some embodiments, an interfering nucleic acid binds directly to a repeat sequence set forth in any one of Tables 1, 2, and 3 (e.g., a microsatellite expansion comprising any one of the sequences set forth in Tables 1, 2, and 3).

In some embodiments, a protein (e.g., a therapeutic protein) modifies eukaryotic initiation factor 2 (eIF2), eukaryotic initiation factor 3 (eIF3), protein kinase R (PKR), p62, LC3 I subunit, LC3 II subunit, or Toll-like receptor 3 (TLR3).

In some embodiments, a protein (e.g., a therapeutic protein) is a dominant-negative variant of protein kinase R (PKR) or a dominant-negative variant of TLR3 protein. In some embodiments, a dominant-negative variant comprises a mutation at amino acid position 296. In some embodiments, the mutation is K296R.

In some embodiments, a therapeutic agent (e.g., a nucleic acid encoding a therapeutic protein, interfering nucleic acid, etc.) is delivered to the subject by a vector. In some embodiments, a vector is a viral vector. In some embodiments, a viral vector is a recombinant adeno-associated virus (rAAV). In some embodiments, an rAAV comprises an AAV8 capsid protein or variant thereof.

In some embodiments, a therapeutic protein is an anti-RAN protein vaccine. In some embodiments, an anti-RAN protein vaccine comprises a peptide antigen comprising an amino acid repeat sequence selected from poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and poly(GSKHREAE) (SEQ ID NO: 267).

In some embodiments, a therapeutic protein is an antibody. In some embodiments, an antibody targets eukaryotic initiation factor 2 (eIF2), eukaryotic initiation factor 3 (eIF3), protein kinase R (PKR), p62, LC3 I subunit, LC3 II subunit, or Toll-like receptor 3 (TLR3). Such antibodies are known in the art (see, e.g., Duffy et al. Cell Immunol. 2007 August; 248(2):103-14. PubMed PMID: 18048020). Those skilled in the art will understand how to make antibodies binding to the enumerated protein targets and to screen for the desired modulation of the functions of the target proteins.

In some embodiments, an antibody is an anti-RAN protein antibody. In some embodiments, an anti-RAN protein antibody targets any one or more of poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and poly(GSKHREAE) (SEQ ID NO: 267). In some embodiments, an anti-RAN protein antibody specifically binds to the poly-amino acid repeat of the RAN protein. In some embodiments, an anti-RAN protein antibody specifically binds to the C-terminus of the RAN protein. In some embodiments, an anti-RAN protein antibody is a monoclonal antibody. In some embodiments, an anti-RAN protein antibody is a polyclonal antibody. In some embodiments, anti-RAN antibodies are generated with binding activity to newly identified RAN proteins occurring in the RAN protein-associated neurological disease (i.e., AD) which are predicted by the sequences of the novel enriched repeat expansion mutations. In some embodiments, the loci comprise known risk factors for the RAN protein-associated neurological disease, now identified as containing novel repeat-expansion mutations capable of producing one or more types of RAN proteins.

In some embodiments, the loci include the LRP8 gene and/or the CASP8 gene. In some embodiments, the LRP8 gene repeat-expansion motif comprises a (sense•antisense) GGGGCA•TGCCCC (SEQ ID NO: 1) repeat motif which encodes proteins containing proline-arginine (PR), glycine-arginine (GR), glycine-aspartic acid (GD), glycine-threonine (GT), valine-proline (VP) and serine-proline (SP) dipeptide repeat motifs from the sense and antisense transcripts. In some embodiments, repeat-expansion mutations in the CASP8 loci comprise a GAGAGG•CCTCTC (SEQ ID NO: 2) repeat motif that can produce novel RAN proteins from sense and antisense transcripts including glycine-arginine (GR), glycine-glutamic acid (GE), arginine-glutamic acid (RE), serine-proline (SP), leucine-proline (LP) and leucine-serine (LS) dipeptide repeat motifs. In some embodiments, repeat-expansion mutations at the C2orf80 locus comprise a GAGAGG repeat motif that can produce novel RAN proteins include glycine-arginine (GR), glycine-glutamic acid (GE), arginine-glutamic acid (RE), serine-proline (SP), leucine-proline (LP) and leucine-serine (LS) dipeptide repeat motifs.

In some embodiments, the loci include the GREB1 gene. In some embodiments, repeat-expansion mutations in the GREB1 loci comprise a GGGGCA repeat motif that can produce novel RAN proteins from sense and antisense transcripts including glycine-arginine (GR), glycine-alanine (GA), glycine-glutamine (GQ), proline-alanine (PA), leucine-proline (LP) and cysteine-proline (CP) dipeptide repeat motifs.

In some embodiments, methods described by the disclosure further comprise administering a second therapeutic agent to the subject (e.g., a therapeutic agent approved by the FDA for treatment of Alzheimer's disease). In some embodiments, a second therapeutic agent is selected from donepezil, galantamine, memantine, rivastigimine, or a combination thereof.

In some embodiments, a biological sample is blood, serum, or cerebrospinal fluid (CSF).

In some embodiments, detection of the one or more RAN proteins comprises performing a binding assay (e.g., an antibody-based binding assay), hybridization assay, immunoblot analysis, Western blot analysis, immunohistochemistry, and/or ELISA (e.g., RCA-based ELISA, rtPCR-based ELISA, etc.). In some embodiments, a hybridization assay comprises contacting a sample with one or more detectable nucleic acid probes (e.g., detectable nucleic acid probes that specifically bind to sequences encoding RAN proteins). In some embodiments, a hybridization assay comprises Fluorescence In Situ Hybridization (FISH) and/or dCas9-based enrichment. In some embodiments, detection of RAN proteins further comprises nucleic acid sequencing, for example Next-Generation Sequencing (NGS), either with or without performing an enrichment step (e.g., dCas9-based enrichment) on the sample.

In some embodiments, the detection of the one or more RAN proteins comprises Next-Generation Sequencing (NGS), either with or without performing an enrichment step (e.g., dCas9-based enrichment) on the sample, using guideRNAs. In some embodiments, the guideRNAs used in the enrichment target NGG protospacer adjacent motifs (PAM) containing repeats. In some embodiments, the guideRNAs used in the enrichment target non-NGG PAM containing repeats. In some embodiments, the non-NGG PAM containing repeats comprise CAG and CTG expansion repeats (e.g., GGGGCC in ALS/FTD and CCTG in DM2). In some embodiments, the guideRNAs used in the enrichment enrich non-NGG PAM containing repeat expansions that are longer (e.g., 1, 2, 3, 4, 5, 6, 7, 8, 9, 10, 11, 12, 13, 14, 15, 20, 25, 30, 35, 40, 45, 50, 55, 60, 70, 80, 90, 100 repeats longer) than the corresponding normal allele. In some embodiments, the guideRNAs used in the enrichment identify multiple repeat expansions simultaneously, including, in some embodiments, sequences with non-NGG PAMs.

In some embodiments, dCas9-based enrichment is performed using a Streptococcus pyogenes-derived dCas9 (spdCas9) molecule. In some embodiments, the dCas9-based enrichment is performed a dCas9 protein selected from the group consisting of: Staphylococcus aureus-, Streptococcus pyogenes-, Campylobacter jejuni-, Corynebacterium diphtheria-, Eubacterium ventriosum-, Streptococcus pasteurianus-, Lactobacillus farciminis-, Sphaerochaeta globus-, an Azospirillum (e.g., strain B510)-, Gluconacetobacter diazotrophicus-, Neisseria cinerea-, Roseburia intestinalis-, Parvibaculum lavamentivorans-, Nitratifractor salsuginis (e.g., strain DSM 16511)-, Campylobacter lari (e.g., strain CF89-12)-, or Streptococcus thermophilus (e.g., strain LMD-9)-derived dCas9 molecule. In still other embodiments, the dCas9 molecule is a mutant of a wild-type Cas9 molecule, e.g., in which the Cas9 nuclease activity is inactivated. In some embodiments, the mutant Cas9 molecule includes a mutation that inactivates a Cas9 nuclease activity, e.g., a mutation in a DNA-cleavage domain of a Cas9 molecule. In some embodiments, the mutant Cas9 molecule includes a mutation that inactivates a Cas9 nuclease activity, e.g., a mutation in a RuvC domain and/or a mutation in a HNH domain.

In some aspects, the disclosure provides a method for diagnosing a RAN protein-associated disease by detecting in a sample obtained from a subject at least one RAN protein; determining that the at least one RAN protein is not transcribed from a C9orf72 locus of the subject; and diagnosing the subject as having a RAN protein-associated disease based on the presence of the at least one RAN protein that was not transcribed from the C9orf72 locus. In some embodiments, the method comprises determining the presence of repeat expansion at one or more specific loci. In some embodiments, determining the presence of repeat expansion at a specific locus is done using repeat prime PCR, long-range PCR, and/or Southern blot and primers that are specific for each locus. In some aspects, the disclosure provides a method of assisting in the diagnosis of a RAN protein-associated disease by performing an assay on a biological sample obtained from a subject to determine whether a RAN protein is present in the biological sample; and identifying the subject as being at risk for a RAN protein-associated disease if the RAN protein is present in the biological sample.

In some embodiments, the sample is central nervous system (CNS) tissue, blood, or cerebrospinal fluid (CSF). In some embodiments, the detecting comprises contacting (e.g., incubating) the sample with an anti-RAN antibody. In some embodiments, the anti-RAN protein antibody targets any one or more of poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and poly(GSKHREAE) (SEQ ID NO: 267). In some embodiments, the detecting comprises performing dCas9-based enrichment on the sample. In some embodiments, the detecting further comprises nucleic acid sequencing. In some embodiments, the sequencing is Next-Generation Sequencing (NGS).

In some aspects, the disclosure provides a method for increasing proteasome activity in a cell, the method comprising administering to the cell an anti-RAN protein antibody in an amount sufficient to reduce RAN protein aggregation in the cell. In some embodiments, an anti-RAN protein antibody targets one or more of the RAN proteins poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and poly(GSKHREAE) (SEQ ID NO: 267).

In some aspects, the disclosure provides a method for vaccinating a subject for a RAN protein disease, the method comprising administering to the subject a peptide antigen that targets one or more RAN proteins. In some embodiments, the peptide antigen targets (e.g., comprise an amino acid sequence encoding) one or more of the RAN proteins poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and poly(GSKHREAE) (SEQ ID NO: 267).

In some embodiments, the cell is a mammalian cell (e.g., a human cell, mouse cell, rat cell, cat cell, dog cell, guinea pig cell, pig cell, monkey cell, etc.). In some embodiments, the cell is a neuronal cell, astrocyte, or glial cell. In some embodiments, the cell contains a gene having a nucleic acid sequence comprising at least 35 repeats of a sequence set forth in any one of Tables 1, 2, and 3. In some embodiments, the anti-RAN protein antibody is a monoclonal antibody.

In some embodiments, administration of the anti-RAN protein antibody results in an increase in proteasome activity in the cell relative to proteasome activity in the cell prior to the administration. In some embodiments, an increase in proteasome activity is indicated by a decrease in P62 subunit levels or inclusions or activity in the cell. In some embodiments, increased proteasome activity can be detected by a diffused signal of proteasome subunits sequestrated by RAN protein, for example by poly(GA), when compared with a typically punctate signal. In some embodiments, an increase in proteasome activity can be measured in protein lysates or in cells using fluorescence-based methods. In some embodiments, improvement in the disease-mediated dysregulation of the extracellular proteasome system can be measured following administration of an anti-RAN antibody.

Also disclosed herein are antibodies and/or antigen-binding fragments that specifically bind to any one or more of polySer, poly(PR), poly(GR), poly(CP), poly(GP); poly(G), poly(A), poly(GA), poly(GD), poly(GE), poly(GQ), poly(GT), poly(L), poly(LP), poly(LPAC) (SEQ ID NO: 260), poly(LS), poly(P), poly(PA), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and/or poly(GSKHREAE) (SEQ ID NO: 267). For example, an antibody or antibody fragment can bind to only one type of RAN protein (e.g., polySer or polyGA), or an antibody or antibody fragment can bind to multiple RAN proteins (e.g., with different affinities). In some embodiments, the antibody or antigen-binding fragment thereof that specifically binds to a RAN protein, and the antibody or antigen-binding fragment comprises a heavy chain variable region (VH) comprising (i) a CDR1 region comprising an amino acid sequence selected from the group consisting of SEQ ID NOs: 117, 119, 121 and 123; (ii) a CDR2 region comprising an amino acid sequence selected from the group consisting of SEQ ID NOs: 125, 127, 129 and 131; and/or (iii) a CDR3 region comprising an amino acid sequence selected from the group consisting of SEQ ID NOs: 133, 135, 137 and 139.

In some embodiments, the antibody or antigen-binding fragment thereof specifically binds to a RAN protein, and the antibody or antigen-binding fragment comprises a light chain variable region (VL) comprising (i) a CDR1 region comprising an amino acid sequence selected from the group consisting of SEQ ID NOs: 118, 120, 122 and 124; (ii) a CDR2 region comprising an amino acid sequence selected from the group consisting of SEQ ID NOs: 126, 128, 130 and 132; and/or (iii) a CDR3 region comprising an amino acid sequence selected from the group consisting of SEQ ID NOs: 134, 136, 138 and 140.

In some embodiments, the antibody or antigen-binding fragment comprises a variable heavy chain amino acid sequence as set forth in SEQ ID NOs: 109, 111, 113 or 115. In some embodiments, the antibody or antigen-binding fragment comprises a variable light chain amino acid sequence as set forth in SEQ ID NOs: 110, 112, 114 or 116.

In some embodiments, the antibody binds polyGA. In some embodiments, the antibody binds polySer. In some embodiments, the antibody binds polyPR.

Also disclosed here are compositions comprising the antibody or antigen-binding fragments disclosed herein, and a pharmaceutically acceptable carrier and/or a pharmaceutically acceptable buffer. In some embodiments, the composition are for use in treating a repeat expansion disease. In some embodiments, the compositions are for use in treating a repeat expansion disease selected from the group consisting of: amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE). In some embodiments, the composition is for use in treating Alzheimer's disease.

Further disclosed herein are isolated nucleic acid molecules encoding the antibody or antigen-binding fragments disclosed herein. Also provided are cells transformed with the disclosed nucleic acids. In some embodiments, the cell is a mammalian cell. In some embodiments, the cell is a cell is a human cell.

Methods of treating a RAN protein-associated disease in a subject are also provided herein. In some embodiments, the method includes administering any one of the antibodies disclosed herein or a combination of two or more antibodies (e.g., 1, 2, 3, 4, 5, 6, 5-10, 10-15, or more antibodies) disclosed herein, wherein the subject has been characterized as having a RAN protein-associated disease by the detection of at least one RAN protein in a biological sample obtained from the subject.

### BRIEF DESCRIPTION OF DRAWINGS

FIGS. 1A-1C show screening for repeat expansions and RNAi foci associated with Alzheimer's disease (AD). FIG. 1A shows a schematic depicting analysis of samples for RAN protein expression using anti-RAN protein antibodies, an RNA foci screening, and dCas9 pull-down enrichment. FIG. 1B shows a schematic depicting dCas9 repeat expansion enrichment. FIG. 1C shows data validating enrichment of C9orf72 G4C2 repeats using sgRNA-dCas9 complexes.

FIGS. 2A-2C show poly(GR) and poly(PR) protein screening data. FIG. 2A shows dot blot screening data for RAN-protein-positive samples. Anti-poly(GR) antibody was used for the screen. FIG. 2B shows dot blot screening data for RAN-protein-positive samples. Anti-poly(Ser) antibody was used for the screen. FIG. 2C shows histological staining of samples for RAN protein (poly(GR) and poly(PR) proteins) and phosphorylated TDP-43 for RAN-positive (top), RAN negative (bottom), and healthy control brain tissue (middle).

FIGS. 3A-3C show immunofluorescence data indicating that RAN protein localization is distinct from typical AD proteins, such as 3R Tau. FIG. 3A shows staining of poly(PR) and poly(GR) is distinct from 3R Tau. FIG. 3B shows that anti-poly(PR) and anti-poly(GR) antibodies do not cross-react with 3R Tau antibody. FIG. 3C shows positive control cells expressing GR₆₀ construct (left) or PR₆₀ construct (right).

FIG. 4 shows Fluorescence In Situ Hybridization (FISH) screening of cells with GC-rich DNA probes. Staining of RNA foci was present in AD cases characterized by RAN protein translation (top) but not in RAN-negative cases (bottom).

FIGS. 5A-5B show effects of RAN protein translation on cell proteasome and autophagy. FIG. 5A shows sequestration of LC3B and 26S subunits by poly(GA) RAN protein in cells transfected with GFP-GA₆₀. FIG. 5B shows reduced proteasome activity in poly(GA) RAN protein expressing cells is rescued by treatment with anti-poly(GA) antibody.

FIG. 6 shows a schematic of molecular pathways controlling autophagy that are affected by expression of RAN proteins.

FIG. 7 shows dCas9-based repeat expansion enrichment and detection (dCas9READ). Favored binding of sgRNA-dCas9 complexes to repeat expansion allows the enrichment and the identification of the repeat and unique flanking sequence.

FIGS. 8A-8D show the enrichment of C9ALS/FTD and DM2 expansions using dCas9READ. FIG. 8A shows qPCR showing enrichment of G4C2 CCTG using dCas9READ.

FIG. 8B shows qPCR showing enrichment of G4C2 expansion mutations using dCas9READ.

FIG. 8C shows the identification of flanking sequences mapping to C9orf72 and CNBP (DM2) loci. FIG. 8D shows total reads showing enrichment of C9orf72 and CNBP from patient versus control DNA. Mean+/−SEM, unpaired two-tailed t-test, **p<0.01, ***p<0.001, ****p<0.0001.

FIGS. 9A-9G show positive RAN protein signal in AD. FIG. 9A shows antibodies used to screen for RAN proteins in AD. FIG. 9B shows an example of dot blot of α-GR screen and quantitation of α-GR signal, unpaired two-tailed t-test. FIG. 9C shows representative positive IHC staining of GR and PR compared to p-Tau, Aβ and pTDP43 in the CA1 region of AD autopsy brains. FIG. 9D shows quantification of PR signal in late-onset AD cases and controls, one-way ANOVA with Tukey analyses for multiple comparisons. FIG. 9E shows α-GR and α-PR staining in T98 cells expressing GR₆₀ and PR60 proteins. FIG. 9F shows α-PR staining in cells expressing 3-repeat tau protein with and without PR60 or GR₆₀. FIG. 9G shows α-GR staining in cells expressing 3-repeat tau protein with and without PR60 or GR₆₀. Mean+/−SEM, **p<0.01, ***p<0.001.

FIGS. 10A-C show RNA foci and accumulation detected in AD cases. FIG. 10A shows RNA foci detected by C4G2 and C4GT DNA probes in AD and quantification of foci in AD samples with positive dot blot signal for α-GR, α-GA, or α-GP. FIG. 10B shows dsRNA signal in the dentate gyrus (DG) region in AD postmortem tissue and quantification comparing cognitive healthy controls, SCA control and AD cases, one-way ANOVA with Turkey analyses for multiple comparisons. FIG. 10C shows dsRNA staining in a control experiment in which tissue was treated with RNAse A, unpaired two-tailed t-test. Data represent mean+/−SEM, *<0.05, **p<0.01.

FIG. 11 shows a Pathology-to-Genetics strategy. Patient tissue is screened using α-RAN antibodies. RAN repeat motifs are used to determine possible repeat motifs for RNA foci screening and sgRNAs for repeat identification using dCas9READ. Novel antibodies against RAN repeats and corresponding unique C-terminal regions will be used to confirm putative expansion mutations and to examine pathology.

FIGS. 12A-12B show CASP8 and ADARB2 expansion loci. FIG. 12A shows CASP8 repeat sequence confirmed by Sanger sequencing of long-range PCR products. FIG. 12B shows expanded and normal alleles at the ADARB2 locus in LOAD cases and non-AD controls. Each lane represents individual AD patients or control samples. Yellow asterisks indicate allele size reported in the reference genome. Red asterisks indicate expanded alleles.

FIGS. 13A-13E show immunofluorescence data validating generated anti-RAN antibodies in transfected cells expressing recombinant proteins. FIG. 13A shows the validation of an anti-polyER antibody. HEK293T cells were transfected with either 3×Flag-(ER)30 or control plasmids. FIG. 13B shows the validation of an anti-polyEG antibody. HEK293T cells were transfected with either 3×Flag-(EG)30 or control plasmids. FIG. 13C shows the validation of an anti-polyLS antibody. HEK293T cells were transfected with either 3×Flag-(LS)30 or control plasmids. FIG. 13D shows the validation of an anti-GAGAGG-ASF1 antibody. HEK293T cells were transfected with either CMV-3×Flag-ASF2 or control plasmids. FIG. 13E shows the validation of an anti-GAGAGG-ASF2 antibody. HEK293T cells were transfected with either CMV-3×Flag-ASF2 or control plasmids.

### DETAILED DESCRIPTION

In some aspects, the disclosure relates to methods and compositions that are useful for the diagnosis and/or treatment of subjects having, or at risk of developing, diseases (e.g., neurological diseases) associated with RAN protein expression, translation, and/or accumulation. In some embodiments, the disease associated with RAN protein expression, translation, and/or accumulation is selected from the group consisting of: amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE). In some embodiments, the neurological disease associated with RAN proteins is Alzheimer's Disease (AD).

In some aspects, the disclosure relates to methods for the diagnosis and/or treatment of subjects having or at risk of developing a disease (e.g., neurological disease) associated with RAN protein expression, translation, and/or accumulation. The disclosure is based, in part, on the identification of certain patients that are characterized by expression and accumulation of certain RAN proteins (e.g., poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and/or poly(GSKHREAE) (SEQ ID NO: 267).

Aspects of the disclosure relate to certain repeat-associated non-ATG (RAN) proteins (e.g., polySerine [polySer], poly(Proline-Arginine) [poly(PR)], and poly(Glycine-Arginine) [poly(GR)]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and/or poly(GSKHREAE) (SEQ ID NO: 267)) which are expressed from a genetic locus of a subject that is not C9orf72, and are detectable in biological samples of subjects having or suspected of having Alzheimer's disease (AD) or another disease (e.g., neurological disease) associated with RAN protein expression, translation, and/or accumulation. Biological samples can be any specimen derived or obtained from a subject having or suspected of having a disease (e.g., neurological disease) associated with RAN proteins expression, translation, and/or accumulation, for example AD. In some embodiments, the biological sample is blood, serum (e.g., plasma from which the clotting proteins have been removed) or cerebrospinal fluid (CSF). In some embodiments, a biological sample is a tissue sample, for example central nervous system (CNS) tissue, such as brain tissue or spinal cord tissue. The skilled artisan will recognize other biological samples, such as cells (e.g., brain cells, neuronal cells, skin cells, etc.) suitable for methods described by the disclosure.

A “subject having or suspected of having a disease (e.g., neurological diseases) associated with RAN protein expression, translation, and/or accumulation” generally refers to a subject exhibiting one or more signs and symptoms of a neurodegenerative disease, including but not limited to memory deficit (e.g., short term memory loss), confusion, deficiencies of executive functions (e.g., attention, planning, flexibility, abstract thinking, etc.), loss of speech, degeneration or loss of motor skills, etc., or a subject having or being identified as having one or more genetic mutations associated with RAN protein expression, translation, and/or accumulation.

A “subject having or suspected of having Alzheimer's disease” can be a subject exhibiting one or more signs and symptoms of AD, including but not limited to memory deficit (e.g., short term memory loss), confusion, deficiencies of executive functions (e.g., attention, planning, flexibility, abstract thinking, etc.), loss of speech, degeneration or loss of motor skills, etc., or a subject having or being identified as having one or more genetic mutations associated with AD, for example mutations in specific genes including apolipoprotein (APP), presenillin genes (PSEN1 and PSEN2), or tau protein. In some embodiments, a subject having or suspected of having AD is characterized by the accumulation of β-amyloid (Aβ) peptides and hyper-phosphorylated tau protein throughout brain tissue of the subject. In some embodiments, a subject has been diagnosed as having AD by a medical professional, according to the NINCDS-ADRDA Alzheimer's Criteria, as described by McKhann et al. (1984) “Clinical diagnosis of Alzheimer's disease: report of the NINCDS-ADRDA Work Group under the auspices of Department of Health and Human Services Task Force on Alzheimer's Disease”. Neurology. 34 (7): 939-44. A subject can be a mammal (e.g., human, mouse, rat, dog, cat, or pig). In some embodiments, a subject is a non-human animal, for example a mouse, rat, guinea pig, cat dog, horse, camel, etc. In some embodiments, the subject is a human.

### RAN Proteins

A “RAN protein (repeat-associated non-ATG translated protein)” is a polypeptide that is translated from sense or antisense RNA sequences bidirectionally transcribed from a repeat expansion mutation in the absence of an AUG initiation codon. RAN protein-encoding sequences can be found in the genome at multiple loci, including but not limited to open reading frame 72 of chromosome 9 (C9orf72), open reading frame 80 of chromosome 2 (C2orf80), LRP8, CASP8, CRNDE, EXOC6B, SV2B, PPML1, ADARB2, GREB1, and MSMO1. The protein associated with C9orf72 is currently poorly characterized but known to be abundant in neurons, especially in the cerebral cortex and motor neurons. C9orf72 protein is believed to localized in presynaptic termini. C9orf72 protein likely impacts transcription, translation and intra-cellular localization of RNA. C9orf72 gene contains a GGGGCC repeat. This hexanucleotide repeat occurs in variable repeat numbers, and small numbers of repeats are not associated with any pathology.

The protein associated with C2orf80 is an uncharacterized protein with expression known to be localized in brain tissue and to a lesser extent testicular tissue. The C2orf80 locus comprises a GAGAGG repeat motif that can produce novel RAN proteins including poly(GR), poly(GE), poly(RE), poly(SP), poly(LP) and poly(LS) dipeptide repeat motifs, depending on the reading frame in which translation is initiated. If translation is initiated in the reading frame yielding the C2orf80 poly(leucine-proline) RAN the C-terminus sequence PCSSPVHLIPDLFVVEFREWSEMDRVGKKGEREEGSLFFQLWALSCNVQSEEKI (SEQ ID NO: 203) is also likely to be translated yielding full length RAN protein with amino acid sequence (LP)ₙPCSSPVHLIPDLFVVEFREWSEMDRVGKKGEREEGSLFFQLWALSCNVQSEEKI (SEQ ID NO: 204) (where n is the number of LP repeats incorporated). If translation is initiated in the reading frame yielding the C2orf80 poly(serine-proline) RAN the C-terminus sequence LLLPCPSDS (SEQ ID NO: 205) is also likely to be translated yielding full length RAN protein with amino acid sequence (SP)ₙLLLPCPSDS (SEQ ID NO: 206) (where n is the number of SP repeats incorporated). If translation is initiated in the reading frame yielding the C2orf80 poly(serine-leucine) RAN the C-terminus sequence PAPPLSI (SEQ ID NO: 207) is also likely to be translated yielding full length RAN protein with amino acid sequence (SL)ₙPAPPLSI (SEQ ID NO: 208) (where n is the number of SL repeats incorporated). If translation is initiated in the reading frame yielding the C2orf80 poly(glycine-glutamate) RAN the C-terminus sequence GDFKQEKRKLLLLREGSRIETFGIQKLIQTFSSTCLFASTE (SEQ ID NO: 209) is also likely to be translated yielding full length RAN protein with amino acid sequence (GE)ₙGDFKQEKRKLLLLREGSRIETFGIQKLIQTFSSTCLFASTE (SEQ ID NO: 210) (where n is the number of GE repeats incorporated). If translation is initiated in the reading frame yielding the C2orf80 poly(glycine-arginine) RAN then it is unlikely that additional amino acids will be translated so the full length RAN protein would simply be (GR)ₙ (SEQ ID NO: 27) (where n is the number of GR repeats incorporated). If translation is initiated in the reading frame yielding the C2orf80 poly(arginine-glutamate) RAN the C-terminus sequence TLSRKKENYYC (SEQ ID NO: 212) is also likely to be translated yielding full length RAN protein with amino acid sequence (RE)ₙTLSRKKENYYC (SEQ ID NO: 213) (where n is the number of RE repeats incorporated).

LRP8 (Low-density lipoprotein receptor-related protein 8), amongst other known functions, serves as key component of the Reelin pathway governing neuronal layering of the forebrain during brain development. The LRP8 gene contains the repeat-expansion motif comprising a (sense•antisense) GGGGCA•TGCCCC (SEQ ID NO: 1) repeat motif which can encode RAN proteins containing poly(PR), poly(GR), poly(GD), poly(GT), poly(VP), and poly(SP) dipeptide repeat motifs from the sense and antisense transcripts depending on the reading frame initiated. If translation is initiated in the reading frame yielding the LRP8 poly(glycine-alanine) RAN the C-terminus sequence DSTSRKALGPLLSLAPSCQAPSIPKPCHNPMLQEVFLQPTPAHLPPS (SEQ ID NO: 214) is also likely to be translated yielding full length RAN protein with amino acid sequence (GA)ₙDSTSRKALGPLLSLAPSCQAPSIPKPCHNPMLQEVFLQPTPAHLPPS (SEQ ID NO: 215) (where n is the number of GA repeats incorporated). If translation is initiated in the reading frame yielding the LRP8 poly(glycine-glutamine) RAN the C-terminus sequence IPPPEKPLGPSSALPPPAKPPVSPSPATIPCSRRSSSSPPQPTSHPPDSDVLSPAKPCDLEEVI FLLCKWGMRTTCLTG (SEQ ID NO: 216) is also likely to be translated yielding full length RAN protein with amino acid sequence (GQ)ₙIPPPEKPLGPSSALPPPAKPPVSPSPATIPCSRRSSSSPPQPTSHPPDSDVLSPAKPCDL EEVIFLLCKWGMRTTCLTG (SEQ ID NO: 217) (where n is the number of GQ repeats incorporated). If translation is initiated in the reading frame yielding the LRP8 poly(glycine-arginine) RAN the C-terminus sequence FHLQKSPWAPPQPCPLLPSPQYPQALPQSHAPGGLPPAHPSPPPTLLTQMFCLLLSRVTW RKSFFSSVNGG (SEQ ID NO: 218) is also likely to be translated yielding full length RAN protein with amino acid sequence (GR)ₙFHLQKSPWAPPQPCPLLPSPQYPQALPQSHAPGGLPPAHPSPPPTLLTQMFCLLLSR VTWRKSFFSSVNGG (SEQ ID NO: 219) (where n is the number of GR repeats incorporated). If translation is initiated in the reading frame yielding the LRP8 poly(cysteine-proline) RAN the C-terminus sequence PSPS LPSPHAVSKWIPDTKTPTGVVPEVRPVNLGPRALPVPLKARVWVCS GAELKAAKL TGKVSPVIRDVQGYGWGRGDSHLW (SEQ ID NO: 220) is also likely to be translated yielding full length RAN protein with amino acid sequence (CP)ₙPSPSLPSPHAVSKWIPDTKTPTGVVPEVRPVNLGPRALPVPLKARVWVCSGAELKA AKLTGKVSPVIRDVQGYGWGRGDSHLW (SEQ ID NO: 221) (where n is the number of CP repeats incorporated). If translation is initiated in the reading frame yielding the LRP8 poly(proline-alanine) RAN the C-terminus sequence RLFPLPMLSPNGSLTPRLQLG (SEQ ID NO: 222) is also likely to be translated yielding full length RAN protein with amino acid sequence (PA)ₙRLFPLPMLSPNGSLTPRLQLG (SEQ ID NO: 223) (where n is the number of PA repeats incorporated). If translation is initiated in the reading frame yielding the LRP8 poly(leucine-proline) RAN the C-terminus sequence QPVSSLSPCCLQMDP (SEQ ID NO: 224) is also likely to be translated yielding full length RAN protein with amino acid sequence (LP)ₙQPVSSLSPCCLQMDP (SEQ ID NO: 225) (where n is the number of LP repeats incorporated).

CASP8 (capsase-8) is expressed in a wide variety of tissues, and serves as the most upstream protease in the cascade activation of caspases driving TNFRSF6/FAS mediated and/or TNFRSF1A induced cell death. The CASP8 gene comprises a GAGAGG•CCTCTC (SEQ ID NO: 2) repeat motif that can produce novel RAN proteins from sense and antisense transcripts including poly(GR), poly(GE), poly(RE), poly(SP), poly(LP), and poly(LS) dipeptide repeat motifs, depending on the reading frame in which translation is initiated. If translation is initiated in the reading frame yielding the CASP8 poly(leucine-serine) RAN the C-terminus sequence PSPSPSPSPRLPLPLMPS QSWTVLLPSRLTATSLPDSPASACRVPAIAGARRHA (SEQ ID NO: 226) is also likely to be translated yielding full length RAN protein with amino acid sequence (LS)ₙPSPSPSPSPRLPLPLMPSQSWTVLLPSRLTATSLPDSPASACRVPAIAGARRHA (SEQ ID NO: 227) (where n is the number of LS repeats incorporated). If translation is initiated in the reading frame yielding the CASP8 poly(leucine-proline) RAN the C-terminus sequence PVSLSLSLSPSPSPSHAEPKLDGTAAISAHCNLPA (SEQ ID NO: 228) is also likely to be translated yielding full length RAN protein with amino acid sequence (LP)ₙPVSLSLSLSPSPSPSHAEPKLDGTAAISAHCNLPA (SEQ ID NO: 229) (where n is the number of LP repeats incorporated). If translation is initiated in the reading frame yielding the CASP8 poly(proline-serine) RAN the C-terminus sequence PRLPLPLPLPVSLSLSCRAKAGRYCCHLGSLQPPCLILLPQPAECLRLQARAATPDWFSFF FWWRWGFAVLAGLVSSS (SEQ ID NO: 230) is also likely to be translated yielding full length RAN protein with amino acid sequence (PS)ₙPRLPLPLPLPVSLSLSCRAKAGRYCCHLGSLQPPCLILLPQPAECLRLQARAATPDW FSFFFWWRWGFAVLAGLVSSS (SEQ ID NO: 231) (where n is the number of PS repeats incorporated). If translation is initiated in the reading frame yielding the CASP8 poly(glycine-arginine) RAN the C-terminus sequence GVKFLSINVMPTVLSSCGL (SEQ ID NO: 232) is also likely to be translated yielding full length RAN protein with amino acid sequence (GR)ₙGVKFLSINVMPTVLSSCGL (SEQ ID NO: 233) (where n is the number of GR repeats incorporated). If translation is initiated in the reading frame yielding the CASP8 poly(arginine-glutamate) RAN the C-terminus sequence RGQILIYQCYAHCALQLWSVNYCGIT (SEQ ID NO: 234) is also likely to be translated yielding full length RAN protein with amino acid sequence (RE)ₙRGQILIYQCYAHCALQLWSVNYCGIT (SEQ ID NO: 235) (where n is the number of RE repeats incorporated). If translation is initiated in the reading frame yielding the CASP8 poly(glycine-glutamate) RAN the C-terminus sequence GSNSYLSMLCPLCSPAVVCELLWYNVTVQISLFRGFDHDL (SEQ ID NO: 236) is also likely to be translated yielding full length RAN protein with amino acid sequence (GE)ₙGSNSYLSMLCPLCSPAVVCELLWYNVTVQISLFRGFDHDL (SEQ ID NO: 259) (where n is the number of GE repeats incorporated).

The Colorectal Neoplasia Differentially Expressed (CRNDE) locus is believed to be transcribed into multiple transcript variants, some of which may function as non-coding RNAs. One transcript variant encodes a putative short protein localized to the nucleus. CRNDE expression is increased in proliferating tissues, such as colorectal adenomas and adenocarcinomas. The CRNDE gene includes a GGGGGC (G5C) repeat motif that, depending on the reading frame in which translation is initiated, can produce novel RAN proteins from sense and antisense transcripts including poly(Glycine) [polyGly] and poly(Proline) [polyPro] proteins, as well as the dipeptide repeats poly(GA), poly(GR), poly(PA) and poly(PR). Based on the nucleic acid sequence that is typically located 3′ of the repeat motif, if translation is initiated in the reading frame yielding the CRNDE polyGly RAN the C-terminus sequence RKRGTAGVAG (SEQ ID NO: 3) is also likely to be translated, yielding full length RAN protein with amino acid sequence (G)ₙRKRGTAGVAG (SEQ ID NO: 4) (where n is the number of glycines incorporated). Were translation to be initiated in the reading frame yielding the CRNDE polyPro RAN, the C-terminus sequence RGLFVGCFFNFFNPFSCTVFFLVSGAGETPARY (SEQ ID NO: 5) is also likely to be translated yielding full length RAN protein with amino acid sequence (P)ₙRGLFVGCFFNFFNPFSCTVFFLVSGAGETPARY (SEQ ID NO: 6) (where n is the number of prolines incorporated). If translation is initiated in the reading frame yielding the CRNDE poly(GA) RAN, the C-terminus sequence GVGGESAGLPEWQDDVMRMSV (SEQ ID NO: 7) is also likely to be translated yielding full length RAN protein with amino acid sequence (GA)ₙGVGGESAGLPEWQDDVMRMSV (SEQ ID NO: 8) (where n is the number of glycine-alanine repeats incorporated). If translation is initiated in the reading frame yielding the CRNDE poly(GR) RAN, the C-terminus sequence GWGEKARDCRSGRMM (SEQ ID NO: 9) is also likely to be translated yielding full length RAN protein with amino acid sequence (GR)ₙGWGEKARDCRSGRMM (SEQ ID NO: 10) (where n is the number of glycine-arginine repeats incorporated). If translation is initiated in the reading frame yielding the CRNDE poly(PR) RAN, the C-terminus sequence PVACLLVVFLIFLTPFLVLSSFWCQGLERLLQDIEAFRMYGSV (SEQ ID NO: 11) is also likely to be translated yielding full length RAN protein with amino acid sequence (PR)ₙPVACLLVVFLIFLTPFLVLSSFWCQGLERLLQDIEAFRMYGSV (SEQ ID NO: 12) (where n is the number of proline-arginine repeats incorporated). If translation is initiated in the reading frame yielding the CRNDE poly(PA) RAN, the C-terminus sequence PWLVCWLFF (SEQ ID NO: 13) is also likely to be translated yielding full length RAN protein with amino acid sequence (PA)ₙ PWLVCWLFF (SEQ ID NO: 14) (where n is the number of proline-alanine repeats incorporated).

EXOC6B (Exocyst complex component 6B) is one portion of an exocyst complex involved in the docking of exocytic vesicles with fusion sites on the plasma membrane. The EXOC6B gene includes a GGGGCA (G4CA) repeat motif that, depending on the reading frame in which translation is initiated, can produce novel RAN proteins from sense and antisense transcripts including the dipeptide repeats poly(glycine-alanine) [poly(GA)], poly(glycine-glutamine) [poly(GQ)], poly(glycine-arginine) [poly(GR)], poly(cysteine-proline) [poly(CP)], poly(proline-alanine) [poly(PA)], and poly(leucine-proline) [poly(LP)]. If translation is initiated in the reading frame yielding the EXOC6B poly(glycine-alanine) RAN the C-terminus sequence GGRRREEVVLIPHFWLPEKALWQRDAGASKLKVQSACTEGKN (SEQ ID NO: 15) is also likely to be translated yielding full length RAN protein with amino acid sequence (GA)ₙGGRRREEVVLIPHFWLPEKALWQRDAGASKLKVQSACTEGKN (SEQ ID NO: 16) (where n is the number of glycine-alanine repeats incorporated). If translation is initiated in the reading frame yielding the EXOC6B poly(glycine-glutamine) RAN the C-terminus sequence GAGGEKRWY (SEQ ID NO: 17) is also likely to be translated yielding full length RAN protein with amino acid sequence (GQₙ GAGGEKRWY (SEQ ID NO: 18) (where n is the number of glycine-glutamine repeats incorporated). If translation is initiated in the reading frame yielding the EXOC6B poly(glycine-arginine) RAN the C-terminus sequence GQEERRGGINSPFLASRESPLAKRCRC (SEQ ID NO: 19) is also likely to be translated yielding full length RAN protein with amino acid sequence (GR)ₙGQEERRGGINSPFLASRESPLAKRCRC (SEQ ID NO: 20) (where n is the number of glycine-arginine repeats incorporated). If translation is initiated in the reading frame yielding the EXOC6B poly(proline-cysteine) RAN the C-terminus sequence PSSPQLITHGSWCINTSASLPTRKEDGVEYL (SEQ ID NO: 22) is also likely to be translated yielding full length RAN protein with amino acid sequence (PC)ₙPSSPQLITHGSWCINTSASLPTRKEDGVEYL (SEQ ID NO: 23) (where n is the number of proline-cysteine repeats incorporated). If translation is initiated in the reading frame yielding the EXOC6B poly(proline-alanine) RAN the C-terminus sequence PVVLSS (SEQ ID NO: 24) is also likely to be translated yielding full length RAN protein with amino acid sequence (PA)ₙPVVLSS (SEQ ID NO: 25) (where n is the number of proline-alanine repeats incorporated). If translation is initiated in the reading frame yielding the EXOC6B poly(proline-lysine) RAN then it is unlikely that additional amino acids will be translated so the full length RAN protein would simply be (PL)ₙ (SEQ ID NO: 26) (where n is the number of proline-lysine repeats incorporated).

SV2B (Synaptic vesicle glycoprotein 2B) is believed to play a role in the control of secretion from neural and endocrine cells. In the former it is a component of the pathology of botulism serving as a receptor for C. botulinum neurotoxin. The SV2B gene also comprises the G4CA repeat motif and thus is capable of producing the RAN proteins described for the same motif in the EXOC6B gene above. If translation is initiated in the reading frame yielding the SV2B poly(glycine-arginine) RAN then it is unlikely that additional amino acids will be translated so the full length RAN protein would simply be (GR)ₙ (SEQ ID NO: 27) (where n is the number of glycine-arginine repeats incorporated). If translation is initiated in the reading frame yielding the SV2B poly(glycine-alanine) RAN the C-terminus sequence GDSNTTSAKSQDTASLQM (SEQ ID NO: 28) is also likely to be translated yielding full length RAN protein with amino acid sequence (GA)ₙGDSNTTSAKSQDTASLQM (SEQ ID NO: 29) (where n is the number of glycine-alanine repeats incorporated). If translation is initiated in the reading frame yielding the SV2B poly(glycine-glutamine) RAN the C-terminus sequence GTVTQHLPRVKTQPLCKCRQAMLRCV (SEQ ID NO: 30) is also likely to be translated yielding full length RAN protein with amino acid sequence (GQₙGTVTQHLPRVKTQPLCKCRQAMLRCV (SEQ ID NO: 31) (where n is the number of glycine-glutamine repeats incorporated). If translation is initiated in the reading frame yielding the SV2B poly(proline-alanine) RAN then it is unlikely that additional amino acids will be translated so the full length RAN protein would simply be (PA)ₙ (SEQ ID NO: 32) (where n is the number of proline-alanine repeats incorporated). If translation is initiated in the reading frame yielding the SV2B poly(proline-lysine) RAN the C-terminus sequence PSHNSLTLVSSLTLPLDTIGTDPQQSA (SEQ ID NO: 33) is also likely to be translated yielding full length RAN protein with amino acid sequence (PL)ₙPSHNSLTLVSSLTLPLDTIGTDPQQSA (SEQ ID NO: 34) (where n is the number of proline-lysine repeats incorporated). If translation is initiated in the reading frame yielding the SV2B poly(proline-cysteine) RAN the C-terminus sequence PHIIL is also likely to be translated yielding full length RAN protein with amino acid sequence (PC)ₙ PHIIL (SEQ ID NO: 35) (where n is the number of proline-cysteine repeats incorporated).

Methylsterol monooxygenase 1, the protein encoded by the MSMO1 locus, is an enzyme localized to the endoplasmic reticulum where it is part of a catalytic pathway removing methyl groups from 4,4-dimethylzymosterol, thus contributing to zymosterol biosynthesis, part of steroid biosynthesis. The MSMO1 gene also comprises the G5C repeat motif and thus is capable of producing the RAN proteins described for the same motif at the CRNDE locus above. If translation is initiated in the reading frame yielding the MSMO1 poly(proline-alanine) RAN the C-terminus sequence PGHSSSSTTIATTPGRSLPM (SEQ ID NO: 36) is also likely to be translated yielding full length RAN protein with amino acid sequence (PA)ₙPGHSSSSTTIATTPGRSLPM (SEQ ID NO: 37) (where n is the number of proline-alanine repeats incorporated). Were translation to be initiated in the reading frame yielding the MSMO1 polyPro RAN the C-terminus sequence GTHRPAQQLQQHLGVLCPCSEVKVLGAELSLDVQSF (SEQ ID NO: 38) is also likely to be translated yielding full length RAN protein with amino acid sequence (P)ₙGTHRPAQQLQQHLGVLCPCSEVKVLGAELSLDVQSF (SEQ ID NO: 39) (where n is the number of prolines incorporated). If translation is initiated in the reading frame yielding the MSMO1 poly(proline-arginine) RAN the C-terminus sequence ALIVQHNNCNNTWAFSAHVARSRSWEPNSPLMFNLFKSFPAFISHLQNDDNRI (SEQ ID NO: 40) is also likely to be translated yielding full length RAN protein with amino acid sequence (PR)ₙALIVQHNNCNNTWAFSAHVARSRSWEPNSPLMFNLFKSFPAFISHLQNDD NRI (SEQ ID NO: 41) (where n is the number of proline-arginine repeats incorporated). If translation is initiated in the reading frame yielding the MSMO1 poly(glycine-arginine) RAN the C-terminus sequence GLDAGLCSSKAQFTPSLNIKILCTGV (SEQ ID NO: 42) is also likely to be translated yielding full length RAN protein with amino acid sequence (GR)ₙGLDAGLCSSKAQFTPSLNIKILCTGV (SEQ ID NO: 43) (where n is the number of glycine-arginine repeats incorporated). If translation is initiated in the reading frame yielding the MSMO1 polyGly RAN, the C-terminus sequence LTQVFAALKLSLHHLSTLKYCVLGFNNKTVQM (SEQ ID NO: 44) is also likely to be translated yielding full length RAN protein with amino acid sequence (G)ₙLTQVFAALKLSLHHLSTLKYCVLGFNNKTVQM (SEQ ID NO: 45) (where n is the number of glycines incorporated). Finally, If translation is initiated in the reading frame yielding the MSMO1 poly(glycine-alanine) RAN, it is unlikely that additional amino acids will be translated so the full length RAN protein would simply be (GA)ₙ (SEQ ID NO: 46) (where n is the number of glycine-alanine repeats incorporated).

Protein phosphatase 1L, the protein encoded by the PPM1L locus, is a magnesium or manganese-requiring phosphatase, involved in signaling pathways. The protein downregulates apoptosis signal-regulating kinase 1, a protein involved in apoptosis following cytotoxic stresses. This protein is an endoplasmic reticulum transmembrane protein that also helps regulate ceramide transport from the ER to the Golgi apparatus. The PPM1L gene comprises the GGGGAA repeat motif. If translation is initiated in the reading frame yielding the PPM1L poly(phenylalanine-proline) RAN the C-terminus sequence PLPLPLPFPLPRSLPLPLPSPPLFDRVSLVTQSGVHWHNLGSLQPPPPRFR (SEQ ID NO: 237) is also likely to be translated yielding full length RAN protein with amino acid sequence (FP)ₙ LPLPLPFPLPRSLPLPLPSPPLFDRVSLVTQSGVHWHNLGSLQPPPPRFR (SEQ ID NO: 238) (where n is the number of phenylalanine-proline repeats incorporated). Were translation to be initiated in the reading frame yielding the PPM1L poly(serine-proline) RAN the C-terminus sequence FLSPSPFLSPSPFPFPFPFPFPFPVPFPFPSPPLPFLTESHWSPSLECTGTILAHCNLRLPGSG DC (SEQ ID NO: 239) is also likely to be translated yielding full length RAN protein with amino acid sequence (SP)ₙFLSPSPFLSPSPFPFPFPFPFPFPVPFPFPSPPLPFLTESHWSPSLECTGTILAHCNLRLP GSGDC (SEQ ID NO: 240) (where n is the number of serine-proline repeats incorporated). If translation is initiated in the reading frame yielding the PPM1L poly(leucine-proline) RAN the C-terminus sequence CPFPLPLSFPLPLSFPLPLSPSPSPSLSPSPFPSPSPPLPSPF (SEQ ID NO: 241) is also likely to be translated yielding full length RAN protein with amino acid sequence (LP)ₙ CPFPLPLSFPLPLSFPLPLSPSPSPSLSPSPFPSPSPPLPSPF (SEQ ID NO: 242) (where n is the number of leucine-proline repeats incorporated). If translation is initiated in the reading frame yielding the PPML1 poly(glycine-glutamate) RAN the C-terminus sequence GDREGKKVSSAEYISRLRSHHSKHYCSSDMLKQNSQTLLSLVTSKSK (SEQ ID NO: 243) is also likely to be translated yielding full length RAN protein with amino acid sequence (GE)ₙ GDREGKKVSSAEYISRLRSHHSKHYCSSDMLKQNSQTLLSLVTSKSK (SEQ ID NO: 244) (where n is the number of glycine-glutamate repeats incorporated). If translation is initiated in the reading frame yielding the PPML1 poly(glycine-lysine) RAN, the C-terminus sequence TGRERKFQALNIFQD (SEQ ID NO: 245) is also likely to be translated yielding full length RAN protein with amino acid sequence (GK)ₙ TGRERKFQALNIFQD (SEQ ID NO: 246) (where n is the number of glycine-lysine repeats incorporated). Finally, if translation is initiated in the reading frame yielding the PPML1 poly(glycine-arginine) RAN, the C-terminus sequence GQGGKESFKR (SEQ ID NO: 247) is also likely to be translated yielding full length RAN protein with amino acid sequence (GR)ₙ GQGGKESFKR (SEQ ID NO: 248) (where n is the number of glycine-arginine repeats incorporated).

ADARB2 (Adenosine Deaminase RNA Specific B2) encodes a member of the double-stranded RNA adenosine deaminase family of RNA-editing enzymes, and may play a regulatory role in RNA editing, but appears to lack editing activity itself, preventing the binding of other ADAR enzymes, decreasing the efficiency of these enzymes. The ADARB2 gene comprises a TTTACTCCCCTCTCCCTCCCGGTG (SEQ ID NO: 21) repeat motif. In some embodiments, an ADARB2 gene encodes a RAN protein comprising a poly(GRQRGVNT) (SEQ ID NO: 266) repeat, and/or a poly(GSKHREAE) (SEQ ID NO: 267) repeat. If translation is initiated in the reading frame yielding the ADARB2 poly(FTPLSLPV) (SEQ ID NO: 262) RAN the C-terminus sequence PLPPGAYSPLPPGVYSPLLPGVYSLCPGVYSPASWPSTFCRSCCFHTFCPMGDGLCSVGP (SEQ ID NO: 249) is also likely to be translated yielding full length RAN protein with amino acid sequence (FTPLSLPV)ₙPLPPGAYSPLPPGVYSPLLPGVYSLCPGVYSPASWPSTFCRSCCFHTFCPM GDGLCSVGP (SEQ ID NO: 250) (where n is the number of FTPLSLPV (SEQ ID NO: 268) repeats incorporated). If translation is initiated in the reading frame yielding the ADARB2 poly(LLPSPSRC) (SEQ ID NO: 263) RAN the C-terminus sequence AGLAVFTRSAPWVMDCVLWGPEITQATEQTFSPQEVLAASSSLPASVPALCPQPPSPTAP AASPRTLGKCIPSLGPGTGPVSHVAALDPPSPVLVPHAGQASGAPVCGPPQLVAQHQAC NQLLVNIGPVAFSDTNKSEGSW (SEQ ID NO: 251) is also likely to be translated yielding full length RAN protein with amino acid sequence (LLPSPSRC)ₙAGLAVFTRSAPWVMDCVLWGPEITQATEQTFSPQEVLAASSSLPASVPAL CPQPPSPTAPAASPRTLGKCIPSLGPGTGPVSHVAALDPPSPVLVPHAGQASGAPVCGPP QLVAQHQACNQLLVNIGPVAFSDTNKSEGSW (SEQ ID NO: 252) (where n is the number of LLPSPSRC (SEQ ID NO: 269) repeats incorporated). If translation is initiated in the reading frame yielding the ADARB2 poly(YSPLPPGV) (SEQ ID NO: 264) RAN the C-terminus sequence CLLPSPSRCLLPSASRCLLPSPSRCLLPSSSRCLLPSASRCLLPSASRCLLPVSWCLLPCFL AIYLLPVLLFSHVLPHG (SEQ ID NO: 253) is also likely to be translated yielding full length RAN protein with amino acid sequence (YSPLPPGV)ₙ CLLPSPSRCLLPSASRCLLPSPSRCLLPSSSRCLLPSASRCLLPSASRCLLPVSWCLLPCFL AIYLLPVLLFSHVLPHG (SEQ ID NO: 254) (where n is the number of YSPLPPGV (SEQ ID NO: 270) repeats incorporated). If translation is initiated in the reading frame yielding the ADARB2 poly(HREGEGSK) (SEQ ID NO: 255) RAN then it is unlikely that additional amino acids will be translated so the full length RAN protein would simply be (HREGEGSK)ₙ (SEQ ID NO: 255) (where n is the number of HREGEGSK (SEQ ID NO: 271) repeats incorporated). If translation is initiated in the reading frame yielding the ADARB2 poly(TGRERGVN) (SEQ ID NO: 265) RAN the C-terminus sequence GKHRRRKCRHVRSAPTPMGQRWGCSAPAS QLVGVLQQANHSPSERLGTLPPHLGHGW MQKESRFATVLHSHLCW (SEQ ID NO: 256) is also likely to be translated yielding full length RAN protein with amino acid sequence (TGRERGVN)ₙGKHRRRKCRHVRSAPTPMGQRWGCSAPASQLVGVLQQANHSPSERLG TLPPHLGHGWMQKESRFATVLHSHLCW (SEQ ID NO: 257) (where n is the number of TGRERGVN (SEQ ID NO: 272) repeats incorporated). If translation is initiated in the reading frame yielding the ARARB2 poly(PGGRGE) (SEQ ID NO: 258) RAN then it is unlikely that additional amino acids will be translated so the full length RAN protein would simply be (PGGRGE)ₙ (SEQ ID NO: 258) (where n is the number of PGGRGE (SEQ ID NO: 273) repeats incorporated).

GREB1 (growth regulating estrogen receptor binding 1) is an estrogen-responsive gene that is an early response gene in the estrogen receptor-regulated pathway. It is thought to play an important role in hormone-responsive tissues and cancer, and it encodes the GREB1 protein. The GREB1 gene comprises a GGGGCA repeat motif. If translation is initiated in the reading frame yielding the GREB1 poly(glycine-arginine) RAN, the C-terminus sequence DRMPSVGEGAEG (SEQ ID NO: 128) is also likely to be translated yielding full length RAN protein with amino acid sequence (GR)ₙ DRMPSVGEGAEG (SEQ ID NO: 136) (where n is the number of glycine-arginine repeats incorporated). If translation is initiated in the reading frame yielding the GREB1 poly(glycine-alanine) RAN, the C-terminus sequence GTGCLQWVKVQKGRSEEVGMEEGEEGGGEELRK (SEQ ID NO: 182) is also likely to be translated yielding full length RAN protein with amino acid sequence (GA)ₙ GTGCLQWVKVQKGRSEEVGMEEGEEGGGEELRK (SEQ ID NO: 185) (where n is the number of glycine-alanine repeats incorporated). If translation is initiated in the reading frame yielding the GREB1 poly(glycine-glutamine) RAN, the C-terminus sequence DAFSG (SEQ ID NO: 186) is also likely to be translated yielding full length RAN protein with amino acid sequence (GQ)ₙ DAFSG (SEQ ID NO: 200) (where n is the number of glycine-glutamine repeats incorporated). If translation is initiated in the reading frame yielding the GREB1 poly(proline-alanine) RAN, the C-terminus sequence WARGSLSSSRSPLTSLPWGLPQTQVSPRHTLHLCGASPDGP (SEQ ID NO: 211) is also likely to be translated yielding full length RAN protein with amino acid sequence (PA)ₙ WARGSLSSSRSPLTSLPWGLPQTQVSPRHTLHLCGASPDGP (SEQ ID NO: 274) (where n is the number of proline-alanine repeats incorporated). If translation is initiated in the reading frame yielding the GREB1 poly(leucine-proline) RAN, the C-terminus sequence GHVAHSLPPGLL (SEQ ID NO: 275) is also likely to be translated yielding full length RAN protein with amino acid sequence (LP). GHVAHSLPPGLL (SEQ ID NO: 276) (where n is the number of leucine-proline repeats incorporated). If translation is initiated in the reading frame yielding the GREB1 poly(cysteine-proline) RAN, the C-terminus sequence CLGTWLTLFLQVSFNITSLGTPTDSGLPATHTSPLWCFSRWSLTSHVSNICPSCWAGYS (SEQ ID NO: 277) is also likely to be translated yielding full length RAN protein with amino acid sequence (CP)ₙ CLGTWLTLFLQVSFNITSLGTPTDSGLPATHTSPLWCFSRWSLTSHVSNICPSCWAGYS (SEQ ID NO: 278) (where n is the number of cysteine-proline repeats incorporated).

Generally, RAN proteins comprise expansion repeats of a single amino acid, di-amino acid, tri-amino acid, or quad-amino acid (e.g., tetra-amino acid), termed poly amino acid repeats. For example, “AAAAAAAAAAAAAAAAAAAA” (poly-Alanine) (SEQ ID NO: 47), “LLLLLLLLLLLLLLLLLL” (poly-Leucine) (SEQ ID NO: 48), “SSSSSSSSSSSSSSSSSSSS” (poly-Serine) (SEQ ID NO: 49), or “CCCCCCCCCCCCCCCCCCCC” (poly-Cysteine) (SEQ ID NO: 50) are poly amino acid repeats that are each 20 amino acid residues in length. Examples of di-amino acid RAN proteins include GPGPGPGPGPGPGPGPGPGP (poly-GP) (SEQ ID NO: 51), GAGAGAGAGAGAGAGAGAGA (poly-GA) (SEQ ID NO: 52), GRGRGRGRGRGRGRGRGRGR (poly-GR) (SEQ ID NO: 53), PAPAPAPAPAPAPAPAPAPA (poly-PA) (SEQ ID NO: 54), and PRPRPRPRPRPRPRPRPRPR (poly-PR) (SEQ ID NO: 55). Examples of tetra-amino acid repeats include LPACLPACLPAC (e.g., poly-LPAC) (SEQ ID NO: 56) and QAGRQAGRQAGR (e.g., poly-QAGR) (SEQ ID NO: 57). RAN proteins can have a poly amino acid repeat of at least 20, at least 25, at least 30, at least 40, at least 50, at least 60, at least 70, at least 80, at least 90, at least 100, or at least 200 amino acid residues in length. In some embodiments, a RAN protein has a poly amino acid repeat more than 200 amino acid residues (e.g., 500, 1000, 5000, 10,000, etc.) in length.

Generally, RAN proteins are translated from abnormal repeat expansions (e.g., TCT repeats, hexanucleotide repeats, etc.) of DNA. The disclosure is based, in part, on the identification of microsatellite repeats in certain subjects having a RAN protein-associated disease that is characterized by expression of one or more (e.g., 2, 3, 4, 5, or more) RAN proteins, for example poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), and/or poly(PGGRGE) (SEQ ID NO: 258). In some embodiments, the disease status of a subject having or suspected of having a RAN protein-associated disease is classified by the number and/or type of microsatellite repeats present (e.g., detected) in the subject (e.g., in the genome of a subject or in a gene of the subject). In some embodiments, a subject having less than 10 repeat sequences does not exhibit signs or symptoms of a RAN protein-associated disease characterized by RAN protein translation. In some embodiments, a subject having between 10 and 40 repeats may or may not exhibit one or more signs or symptoms of a RAN protein-associated disease characterized by RAN protein translation. In some embodiments, a subject having more than 40 trinucleotide repeats exhibits one or more signs or symptoms of a RAN protein-associated disease characterized by RAN protein translation. In certain cases, a subject is identified as having a RAN protein-associated disease characterized by large (>100) number of repeats. Microsatellite repeat sequences encoding RAN proteins are generally known. In some embodiments, the RAN protein-associated disease is Alzheimer's disease.

In some embodiments, a subject having or suspected of having a RAN protein-associated disease has one or more microsatellite repeat sequences encoding a poly(PR) RAN protein. Examples of microsatellite repeat sequences encoding poly(PR) proteins include CCTCGT (SEQ ID NO: 58), CCCCGT (SEQ ID NO: 59), CCACGT (SEQ ID NO: 60), CCGCGT (SEQ ID NO: 61), CCTCGC (SEQ ID NO: 62), CCCCGC (SEQ ID NO: 63), CCACGC (SEQ ID NO: 64), CCGCGC (SEQ ID NO: 65), CCTCGA (SEQ ID NO: 66), CCCCGA (SEQ ID NO: 67), CCACGA (SEQ ID NO: 68), CCGCGA (SEQ ID NO: 69), CCTCGG (SEQ ID NO: 70), CCCCGG (SEQ ID NO: 71), CCACGG (SEQ ID NO: 72), CCGCGG (SEQ ID NO: 73), CCTAGA (SEQ ID NO: 74), CCCAGA (SEQ ID NO: 75), CCAAGA (SEQ ID NO: 76), CCGAGA (SEQ ID NO: 77), CCTAGG (SEQ ID NO: 78), CCCAGG (SEQ ID NO: 79), CCAAGG (SEQ ID NO: 80), and CCGAGG (SEQ ID NO: 81).

In some embodiments, a subject having or suspected of having a RAN protein-associated disease has one or more microsatellite repeat sequences encoding a poly(GR) RAN protein. Examples of microsatellite repeat sequences encoding poly(GR) proteins include GGTCGT (SEQ ID NO: 82), GGCCGT (SEQ ID NO: 83), GGACGT (SEQ ID NO: 84), GGGCGT (SEQ ID NO: 85), GGTCGC (SEQ ID NO: 86), GGCCGC (SEQ ID NO: 87), GGACGC (SEQ ID NO: 88), GGGCGC (SEQ ID NO: 89), GGTCGA (SEQ ID NO: 90), GGCCGA (SEQ ID NO: 91), GGACGA (SEQ ID NO: 92), GGGCGA (SEQ ID NO: 93), GGTCGG (SEQ ID NO: 94), GGCCGG (SEQ ID NO: 95), GGACGG (SEQ ID NO: 96), GGGCGG (SEQ ID NO: 97), GGTAGA (SEQ ID NO: 98), GGCAGA (SEQ ID NO: 99), GGAAGA (SEQ ID NO: 100), GGGAGA (SEQ ID NO: 101), GGTAGG (SEQ ID NO: 102), GGCAGG (SEQ ID NO: 103), GGAAGG (SEQ ID NO: 104), and GGGAGG (SEQ ID NO: 105).

Following the enumeration of the possible repeat motifs capable of generating RAN proteins poly(PR) and poly(GR) above one skilled in the art will understand how to derive the possible hexanucleotide repeats that could generate other RAN proteins, for example poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and/or poly(GSKHREAE) (SEQ ID NO: 267).

In some embodiments, a subject having or suspected of having a RAN protein-associated disease has one or more microsatellite repeat sequences encoding a polySer RAN protein. Examples of microsatellite repeat sequences encoding polySer proteins include TCT, TCC, TCA, TCG, AGT, and AGC.

In some aspects, the disclosure relates to the discovery that RAN protein (e.g., poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and/or poly(GSKHREAE) (SEQ ID NO: 267) aggregation patterns are length-dependent. For example, RAN proteins having poly amino acid repeats that are >20, >48, or >80 residues in length aggregate differently in the brain of a subject. Generally, the differential aggregation properties of RAN proteins having different lengths can be used to detect RAN proteins in a biological sample. Longer RAN proteins are found at higher levels in biological samples, such as blood, serum, or CSF. In some embodiments, RAN proteins having poly amino acid repeats >40, >50, >60, >70, or >80 amino acid residues in length are detectable in a biological sample.

### Methods of Detecting RAN Proteins

The disclosure is based, in part, on the discovery that certain biological sample processing methods (e.g., antibody-based capture, hybridization-based assays, dCas9-based enrichment, or combinations thereof) enable the reproducible detection of one or more RAN proteins in a biological sample. In some embodiments of methods described by the disclosure, a sample (e.g., a biological sample) is treated by an antibody-based capture process to isolate one or more RAN proteins within the sample. Typically, the antibody-based capture methods include contacting the sample with one or more (e.g., 2, 3, 4, 5, or more) anti-RAN protein antibodies. In some embodiments, the one or more anti-RAN antibodies are conjugated to a solid support (e.g., a scaffold, resin beads, etc.). In some embodiments, antibody-based capture methods comprise physically separating and/or isolating RAN proteins that have been bound by the anti-RAN antibody(s), for example eluting the RAN proteins by a chromatographic method such as affinity chromatography or ion-exchange chromatography.

A biological sample may be subjected to an antigen retrieval procedure prior to being contacted with an anti-RAN antibody. As used herein, “antigen retrieval” (also referred to as epitope retrieval, or antigen unmasking) refers to a process in which a biological sample (e.g., blood, serum, CSF, etc.) are treated under conditions which expose antigens (e.g., epitopes) that were previously inaccessible to detection agents (e.g., antibodies, aptamers, and other binding molecules) prior to the process. Generally, antigen retrieval methods comprise steps including but not limited to heating, pressure treatment, enzymatic digestion, treatment with reducing agents, treatment with oxidizing agents, treatment with crosslinking agents, treatment with denaturing agents (e.g., detergents, ethanol, acids), or changes in pH, or any combination of the foregoing. Several antigen retrieval methods are known in the art, including but not limited to protease-induced epitope retrieval (PIER) and heat-induced epitope retrieval (HIER). In some embodiments, antigen retrieval procedures reduce the background and increase the sensitivity of detection techniques (e.g., immunohistochemistry (IHC), immuno-blot (such as Western Blot), ELISA, etc.).

Detection of RAN proteins in a biological sample may be performed by Western blot. Western blots generally employ the use of a detection agent or probe to identify the presence of a protein or peptide. In some embodiments, detection of one or more RAN proteins is performed by immunoblot (e.g., dot blot, 2-D gel electrophoresis, Western Blot, etc.), immunohistochemistry (IHC), ELISA (e.g., RCA-based ELISA or rtPCR-based ELISA), label free immunoassays such as surface plasmon resonance bio layer interferometry, immunoquantitative PCR, mass spectrometry such as GC-MS, LC-MS, MALDI-TOF-MS, bead-based immunoassays, immunoprecipitation, immunostaining, or immunoelectrophoresis. In some embodiments, the detection agent is an antibody. In some embodiments, the antibody is an anti-RAN protein antibody, such as anti-polySer, anti-poly(GR), anti-poly(PR), anti-poly(CP), anti-poly(GP), anti-poly(G), anti-poly(A), anti-poly(GA), anti-poly(GD), anti-poly(GE), anti-poly(GQ), anti-poly(GT), anti-poly(L), anti-poly(LP), anti-poly(LPAC) (SEQ ID NO: 260), anti-poly(LS), anti-poly(P), anti-poly(PA), anti-poly(QAGR) (SEQ ID NO: 261), anti-poly(RE), anti-poly(SP), anti-poly(VP), anti-poly(FP), anti-poly(GK), anti-poly(FTPLSLPV) (SEQ ID NO: 262), anti-poly(LLPSPSRC) (SEQ ID NO: 263), anti-poly(YSPLPPGV) (SEQ ID NO: 264), anti-poly(HREGEGSK) (SEQ ID NO: 255), anti-poly(TGRERGVN) (SEQ ID NO: 265), anti-poly(PGGRGE) (SEQ ID NO: 258), anti-poly(GRQRGVNT) (SEQ ID NO: 266), and/or anti-poly(GSKHREAE) (SEQ ID NO: 267) (also referred to as α-polySer, α-poly(PR), α-poly(GR), etc.). In some embodiments, an anti-RAN protein antibody targets (e.g., specifically binds to) the amino acid repeat region (e.g., PRPRPRPRPR (SEQ ID NO: 106), GRGRGRGRGR (SEQ ID NO: 107), SSSSSSSSS (SEQ ID NO: 108), etc.) of a RAN protein. In some embodiments, an anti-RAN protein antibody targets (e.g., specifically binds to) an epitope comprising amino acids in the characteristic reading frame specific C-terminus translated 3′ of the repeated amino acids. In some embodiments, an anti-RAN protein antibody targets (e.g., specifically binds to) a epitope comprising amino acids bridging the C terminus of the amino acid repeat region and the N terminus of the characteristic reading-frame specific C-terminus translated 3′ of the repeated amino acids.

In some embodiments, an anti-RAN antibody targets (e.g., specifically binds to) any portion of a RAN protein that does not comprise the poly amino acid repeat, for example the C-terminus of a RAN protein (e.g., the C-terminus of a poly(GR), poly(PR), polySer, poly(CP), poly(GP), poly(G), poly(A), poly(GA), poly(GD), poly(GE), poly(GQ), poly(GT), poly(L), poly(LP), poly(LPAC) (SEQ ID NO: 260), poly(LS), poly(P), poly(PA), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258) protein), poly(GRQRGVNT) (SEQ ID NO: 266), and/or poly(GSKHREAE) (SEQ ID NO: 267). Examples of anti-RAN antibodies targeting RAN protein poly amino acid repeats are disclosed, for example, in International Application Publication No. WO 2014/159247, the entire content of which is incorporated herein by reference. Examples of anti-RAN antibodies targeting the C-terminus of RAN protein are disclosed, for example, in U.S. Publication No. 2013/0115603, the entire content of which is incorporated herein by reference. In some embodiments, a set (or combination) of anti-RAN antibodies (e.g., a combination of two or more anti-RAN antibodies selected from anti-polySer, anti-poly(GR), anti-poly(PR), anti-poly(CP), anti-poly(GP), anti-poly(G), anti-poly(A), anti-poly(GA), anti-poly(GD), anti-poly(GE), anti-poly(GQ), anti-poly(GT), anti-poly(L), anti-poly(LP), anti-poly(LPAC) (SEQ ID NO: 260), anti-poly(LS), anti-poly(P), anti-poly(PA), anti-poly(QAGR) (SEQ ID NO: 261), anti-poly(RE), anti-poly(SP), anti-poly(VP), anti-poly(FP), anti-poly(GK), anti-poly(FTPLSLPV) (SEQ ID NO: 262), anti-poly(LLPSPSRC) (SEQ ID NO: 263), anti-poly(YSPLPPGV) (SEQ ID NO: 264), anti-poly(HREGEGSK) (SEQ ID NO: 255), anti-poly(TGRERGVN) (SEQ ID NO: 265), anti-poly(PGGRGE) (SEQ ID NO: 258), anti-poly(GRQRGVNT) (SEQ ID NO: 266), and/or anti-poly(GSKHREAE) (SEQ ID NO: 267), is used to detect one or more RAN proteins in a biological sample.

An anti-RAN antibody can be a polyclonal antibody or a monoclonal antibody. Typically, polyclonal antibodies are produced by inoculation of a suitable mammal, such as a mouse, rabbit or goat. Larger mammals are often preferred as the amount of serum that can be collected is greater. An antigen is injected into the mammal. This induces the B-lymphocytes to produce IgG immunoglobulins specific for the antigen. This polyclonal IgG is purified from the mammal's serum. Monoclonal antibodies are generally produced by a single cell line (e.g., a hybridoma cell line). In some embodiments, an anti-RAN antibody is purified (e.g., isolated from serum). In some embodiments, the antigen is 12-20 amino acids. For antibodies against repeat motifs, an antigen is a repeat sequence. For antibodies against C-terminal sequence of a RAN protein, an antigen is a C-terminal specific sequence. In some embodiments, an antigen is a portion of a C-terminal sequence, for example, a fragment of the C-terminal sequences that is 3-5 or 5-10, or more amino acids in length, for example, 6, 7, 8, 9, 10, 11, 12, 13, 14 15, 16, 17, 18, 19, 20, 25, 30, 35, 40, or 50 amino acids in length (e.g., from one of the C-terminal sequences described in this application).

In some embodiments, the disclosure provides methods of producing an antibody, the method comprising administering to the subject a peptide antigen comprising a RAN protein repeat sequence, for example anti-polySer, anti-poly(GR), anti-poly(PR), anti-poly(CP), anti-poly(GP), anti-poly(G), anti-poly(A), anti-poly(GA), anti-poly(GD), anti-poly(GE), anti-poly(GQ), anti-poly(GT), anti-poly(L), anti-poly(LP), anti-poly(LPAC) (SEQ ID NO: 260), anti-poly(LS), anti-poly(P), anti-poly(PA), anti-poly(QAGR) (SEQ ID NO: 261), anti-poly(RE), anti-poly(SP), anti-poly(VP), anti-poly(FP), anti-poly(GK), anti-poly(FTPLSLPV) (SEQ ID NO: 262), anti-poly(LLPSPSRC) (SEQ ID NO: 263), anti-poly(YSPLPPGV) (SEQ ID NO: 264), anti-poly(HREGEGSK) (SEQ ID NO: 255), anti-poly(TGRERGVN) (SEQ ID NO: 265), anti-poly(PGGRGE) (SEQ ID NO: 258), anti-poly(GRQRGVNT) (SEQ ID NO: 266), and/or anti-poly(GSKHREAE) (SEQ ID NO: 267). In some embodiments, the subject is a mammal, for example a non-human primate, rodent (e.g., rat, hamster, guinea pig, etc.). In some embodiments, the subject is a human (e.g., a subject is injected with a peptide antigen for the purposes of eliciting a host antibody response against the peptide antigen, for example a RAN protein). In some embodiments, an antibody is produced by expressing in a cell (e.g., a B-cell, hybridoma cell, etc.) one or more RAN proteins or RAN protein repeat sequences.

Numerous methods may be used for obtaining anti-RAN antibodies. For example, antibodies can be produced using recombinant DNA methods. Monoclonal antibodies may also be produced by generation of hybridomas (see, e.g., Kohler and Milstein (1975) Nature, 256: 495-499) in accordance with known methods. Hybridomas formed in this manner are then screened using standard methods, such as enzyme-linked immunosorbent assay (ELISA; e.g., RCA-based ELISA or rtPCR-based ELISA) and surface plasmon resonance (e.g., OCTET or BIACORE) analysis, to identify one or more hybridomas that produce an antibody that specifically binds with a specified antigen. Any form of the specified antigen (e.g., a RAN protein) may be used as the immunogen, e.g., recombinant antigen, naturally occurring forms, any variants or fragments thereof. One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al. (1991) Nature, 352: 624-628; Marks et al. (1991) J. Mol. Biol., 222: 581-597WO92/18619; WO 91/17271; WO 92/20791; WO 92/15679; WO 93/01288; WO 92/01047; WO 92/09690; and WO 90/02809.

In addition to the use of display libraries, the specified antigen (e.g., one or more RAN proteins) can be used to immunize a non-human animal, e.g., a rodent, e.g., a mouse, hamster, or rat. In one embodiment, the non-human animal is a mouse.

In another embodiment, a monoclonal antibody is obtained from the non-human animal, and then modified, e.g., made chimeric, using recombinant DNA techniques known in the art. A variety of approaches for making chimeric antibodies have been described. See, e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

Antibodies can also be humanized by methods known in the art. For example, monoclonal antibodies with a desired binding specificity can be commercially humanized (Scotgene, Scotland; and Oxford Molecular, Palo Alto, Calif.). Fully humanized antibodies, such as those expressed in transgenic animals are within the scope of the invention (see, e.g., Green et al. (1994) Nature Genetics 7, 13; and U.S. Pat. Nos. 5,545,806 and 5,569,825).

For additional antibody production techniques, see, Antibodies: A Laboratory Manual, Second Edition. Edited by Edward A. Greenfield, Dana-Farber Cancer Institute, ©2014. The present disclosure is not necessarily limited to any particular source, method of production, or other special characteristics of an antibody.

In some embodiments, methods of detecting one or more RAN proteins in a biological sample are useful for monitoring the progress of a disease associated with RAN protein expression, translation, and/or accumulation. In some embodiments, the disease associated with RAN proteins is selected from the group consisting of: amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE). In a specific embodiment, the neurological disease associated with RAN proteins is Alzheimer's Disease (AD). For example, in some embodiments, biological samples are obtained from a subject prior to and after (e.g., 1 week, 2 weeks, 1 month, 6 months, or one year after) commencement of a therapeutic regimen and the amount of RAN proteins detected in the samples is compared. In some embodiments, if the level (e.g., amount) of RAN protein in the post-treatment sample is reduced compared to the pre-treatment level (e.g., amount) of RAN protein, the therapeutic regimen is successful. In some embodiments, the level of RAN proteins in biological samples (e.g., 1, 2, 3, 4, 5, 6, 7, 8, 9, 10, or more samples) of a subject are continuously monitored during a therapeutic regimen (e.g., measured on 1, 2, 3, 4, 5, 6, 7, 8, 9, 10, or more separate occasions).

In some embodiments, a detection agent is an aptamer (e.g., RNA aptamer, DNA aptamer, or peptide aptamer). In some embodiments, an aptamer specifically binds to a RAN protein (e.g., polySer, poly(PR), poly(GR), poly(CP), poly(GP), poly(G), poly(A), poly(GA), poly(GD), poly(GE), poly(GQ), poly(GT), poly(L), poly(LP), poly(LPAC) (SEQ ID NO: 260), poly(LS), poly(P), poly(PA), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and/or poly(GSKHREAE) (SEQ ID NO: 267)).

Aspects of the disclosure relate to nucleic acid hybridization-based methods for identifying the presence of RAN proteins or microsatellite repeat sequences encoding RAN proteins in a biological sample (e.g., a biological sample obtained from a subject). The disclosure is based, in part, on methods for detecting nucleic acid sequences encoding RAN proteins by detectable nucleic acid probes (e.g., fluorophore-conjugated DNA probes). Generally, a “detectable nucleic acid probe” refers to a nucleic acid sequence that specifically binds to (e.g., hybridizes with) a target sequence, and comprises a detectable moiety, for example a fluorescent moiety, radioactive moiety, chemiluminescent moiety, electroluminescent moiety, biotin, peptide tag (e.g., poly-His tag, FLAG-tag, etc.), etc. In some embodiments, the detectable nucleic acid probe comprises a region of complementarity (e.g., a nucleic acid sequence that is the complement of, and capable of hybridizing to) a nucleic acid sequence encoding one or more RAN proteins. A region of complementarity may range from about 2 nucleotides in length to about 100 nucleotides in length (e.g., any number of nucleotides between 2 and 100, inclusive). In some embodiments, a nucleic acid probe comprises a region of complementarity with a sequence set forth in any one of Tables 1, 2, and 3 or a region of complementarity with a repeat sequence comprising multiple repeats of a sequence set forth in any one of Tables 1, 2, and 3. In some embodiments, a detectable nucleic acid probe is a DNA probe. In some embodiments, the DNA probe is conjugated to a fluorophore.

A biological sample may also be contacted with a plurality of detectable nucleic acid probes. The number of nucleic acid probes in a plurality varies. In some embodiments, a plurality of nucleic acid probes comprises between 2 and 100 (2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100) nucleic acid probes. In some embodiments, a plurality comprises more than 100 probes. The nucleic acid probes may be the same or different sequences. In some embodiments, a plurality of detectable nucleic acid probes comprises probes which hybridize to nucleic acid sequences that encode a poly(GR) RAN protein (e.g., repeat sequences set forth in Table 1). In some embodiments, a plurality of detectable nucleic acid probes comprises probes which hybridize to nucleic acid sequences that encode a poly(PR) RAN protein (e.g., repeat sequences set forth in Table 2). In some embodiments, a plurality of detectable nucleic acid probes comprises probes which hybridize to nucleic acid sequences that encode a polySer RAN protein (e.g., repeat sequences set forth in Table 3). In some embodiments, a plurality of detectable nucleic acid probes comprises probes which hybridize to nucleic acid sequences that encode poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and/or poly(GSKHREAE) (SEQ ID NO: 267).

In some embodiments, detectable nucleic acid probes are useful for localization of RAN protein translation by Fluorescence In situ Hybridization (FISH).

Methods for detecting one or more RAN proteins may comprise an enrichment step. “Enrichment” refers to processes which increase the amount and/or concentration of a target nucleic acid in a sample relative to other nucleic acids in a sample. Generally, enrichment may occur by increasing the number of target nucleic acid sequences in a sample (e.g., by amplifying the target sequence, for example by polymerase chain reaction (PCR), etc.), or by decreasing the amount or concentration of non-target nucleic acid sequences in the sample (e.g., by separating or isolating the target nucleic acid sequence from non-target sequences).

In some embodiments, methods described herein comprise a step of enriching a biological sample for nucleic acid sequences (e.g., microsatellite repeat sequences) encoding RAN proteins. In some embodiments, the enrichment comprises contacting the biological sample with 1) a labeled (e.g., biotinylated) dCas9 protein, and 2) one or more single-stranded guide RNA (sgRNAs) that specifically bind to nucleic acid repeat sequences encoding RAN proteins. In some embodiments, the labeled dCas9 protein and the one or more sgRNAs are provided together as a single molecule (e.g., a dCas9-sgRNA complex). In some embodiments after the biological sample with the labeled dCas9 protein and the one or more sgRNAs, the nucleic acid sequences encoding one or more RAN proteins are isolated from the labeled dCas9 protein and the sgRNAs, for example by affinity chromatography, as described by Liu et al. (2017) Cell 170: 1028-1043.

In some embodiments, the detection of the one or more RAN proteins comprises Next-Generation Sequencing (NGS). In some embodiments, an enrichment step (e.g., dCas9-based enrichment) is performed on the sample, using guideRNAs. In some embodiments, the guideRNAs used in the enrichment target NGG protospacer adjacent motifs (PAM) containing repeats. In other embodiments, the guideRNAs used in the enrichment target non-NGG PAM containing repeats. In some embodiments, the non-NGG PAM containing repeats comprise CAG and CTG expansion repeats (e.g., GGGGCC in ALS/FTD and CCTG in DM2). In some embodiments, the guideRNAs used in the enrichment enrich non-NGG PAM containing repeat expansions that are longer (e.g., 1, 2, 3, 4, 5, 6, 7, 8, 9, 10, 11, 12, 13, 14, 15, 20, 25, 30, 35, 40, 45, 50, 55, 60, 70, 80, 90, 100 repeats longer) than the corresponding normal allele. In some embodiments, the guideRNAs used in the enrichment identify multiple repeat expansions simultaneously, including, in some embodiments, sequences with non-NGG PAMs.

### Therapeutic Methods

Methods of treating a disease associated with RAN protein expression, translation, and/or accumulation are also contemplated by the disclosure. In some embodiments, the disease associated with RAN proteins is selected from the group consisting of: amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE). In a specific embodiment, the neurological disease associated with RAN proteins is Alzheimer's Disease (AD). In some embodiments, a subject having been diagnosed with a disease associated with RAN proteins by a method described by the disclosure is administered a therapeutic useful for treating a disease associated with RAN proteins.

To “treat” a disease (e.g., AD) as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host cell, tissue or organ. A therapeutically acceptable amount of an anti-RAN protein antibody may be an amount that is capable of treating a disease, e.g., Alzheimer's disease, by reducing expression and/or aggregation of RAN proteins and/or appearance or number of RNA foci comprising RAN protein-encoding microsatellite repeat sequences. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

A therapeutic useful for treating a disease associated with RAN proteins can be a small molecule, protein, peptide, nucleic acid (e.g., an interfering nucleic acid), or gene therapy vector (e.g., viral vector encoding a therapeutic protein and/or an interfering nucleic acid). Therapeutics useful for treating a disease associated with RAN proteins may target (e.g., reduce expression, activity, accumulation, aggregation, etc.) of a RAN protein or nucleic acid encoding a RAN protein, and/or modulate the activity of another gene or gene product (e.g., protein) that interact with one or more RAN proteins. Examples of genes and gene products that interact with one or more RAN proteins include eukaryotic initiation factor 2 (eIF2), eukaryotic initiation factor 3 (eIF3), protein kinase R (PKR), p62, LC3 I subunit, LC3 II subunit, and Toll-like receptor 3 (TLR3). In some embodiments, a therapeutic agent inhibits expression or activity of one or more of eukaryotic initiation factor 2 (eIF2), eukaryotic initiation factor 3 (eIF3), protein kinase R (PKR), p62, LC3 I subunit, LC3 II subunit, and Toll-like receptor 3 (TLR3).

In some embodiments, the therapeutic agent is a small molecule. In some embodiments, the small molecule inhibits expression or activity of one or more RAN proteins. In some embodiments, a small molecule is an inhibitor of eIF3 (or an eIF3 subunit). Examples of small molecule inhibitors of eIF3 include but are not limited to mTOR inhibitors (e.g., rapamycin, PP242), S6 kinase (S6K) inhibitors, etc.

In some embodiments, the small molecule inhibits expression or activity of eukaryotic initiation factor 2A (eIF2A) or eIF2a. Examples of small molecule inhibitors of eIF2A include but are not limited to salubrinal, Sa1003, ISRIB, etc. In some embodiments, the small molecule in an inhibitor of TARBP2. Examples of TARBP2 inhibitors include anti-TARBP2 antibodies, interfering RNAs (e.g., dsRNA, siRNA, shRNA, miRNA, etc.) that target anti-TARBP2, peptide inhibitors of TARBP2, and small molecule inhibitors of TARBP2. In some embodiments, the small molecule is metformin, also known as N,N-dimethylbiguanide (IUPAC N,N-Dimethylimidodicarbonimidic diamide and CAS 657-24-9), or an alternate bioactive biguanide including chloroguanide [1-[amino-(4-chloroanilino)methylidene]-2-propan-2-yl-guanidine, CAS 500-92-5], Chlorproguanil [1-[Amino-(3,4-dichloroanilino)methylidene]-2-propan-2-ylguanidine, CAS 537-21-3], buformin [N-Butylimidodicarbonimidic diamide, CAS 692-13-7] or Phenformin [2-(N-phenethylcarbamimidoyl)guanidine, CAS 114-86-3] or a pharmaceutically acceptable salt, co-crystal, tautomer, stereoisomer, solvate, hydrate, polymorph, isotopically enriched derivative, or prodrug of any of the biguanides.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. Metformin may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R.0.5H₂O)), and polyhydrates (x is a number greater than 1, e.g., dihydrates (R.2H₂O) and hexahydrates (R.6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred. In some embodiments, the small molecule is buformin, or phenformin.

The therapeutic agent may be an anti-RAN protein antibody. In some embodiments, the anti-RAN protein antibody is an anti-poly-Serine, anti-poly(GR), anti-poly(PR), anti-poly(CP), anti-poly(GP), anti-poly(G), anti-poly(A), anti-poly(GA), anti-poly(GD), anti-poly(GE), anti-poly(GQ), anti-poly(GT), anti-poly(L), anti-poly(LP), anti-poly(LPAC) (SEQ ID NO: 260), anti-poly(LS), anti-poly(P), anti-poly(PA), anti-poly(QAGR) (SEQ ID NO: 261), anti-poly(RE), anti-poly(SP), anti-poly(VP), anti-poly(FP), anti-poly(GK), anti-poly(FTPLSLPV) (SEQ ID NO: 262), anti-poly(LLPSPSRC) (SEQ ID NO: 263), anti-poly(YSPLPPGV) (SEQ ID NO: 264), anti-poly(HREGEGSK) (SEQ ID NO: 255), anti-poly(TGRERGVN) (SEQ ID NO: 265), anti-poly(PGGRGE) (SEQ ID NO: 258), anti-poly(GRQRGVNT) (SEQ ID NO: 266), and/or anti-poly(GSKHREAE) (SEQ ID NO: 267) antibody (also referred to as α-polySer, α-poly(PR), α-poly(GR), etc.). An anti-RAN protein antibody may bind to an extracellular RAN protein, an intracellular RAN protein, or both extracellular and intracellular RAN proteins.

In some embodiments, an anti-RAN protein antibody targets (e.g., specifically binds to) the amino acid repeat region (e.g., PRPRPRPRPR (SEQ ID NO: 106), GRGRGRGRGR (SEQ ID NO: 107), SSSSSSSSS (SEQ ID NO: 108), etc.) of a RAN protein. Examples of anti-RAN antibodies targeting RAN protein poly amino acid repeats are disclosed, for example, in International Application Publication No. WO 2014/159247, the entire content of which is incorporated herein by reference. In some embodiments, an anti-RAN protein antibody targets (e.g., specifically binds to) the amino acid repeat region of one or more RAN proteins selected from the list poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and poly(GSKHREAE) (SEQ ID NO: 267).

In some embodiments, an anti-RAN antibody targets (e.g., specifically binds to) any portion of a RAN protein that does not comprise the poly amino acid repeat, for example the C-terminus of a RAN protein (e.g., the C-terminus of a poly(CP), poly(GP), poly(G), poly(A), poly(GA), poly(GD), poly(GE), poly(GQ), poly(GR), poly(GT), poly(L), poly(LP), poly(LPAC) (SEQ ID NO: 260), poly(LS), poly(P), poly(PA), poly(PR), poly(QAGR) (SEQ ID NO: 261), poly(RE), polySer, poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258) protein, poly(GRQRGVNT) (SEQ ID NO: 266), and/or poly(GSKHREAE) (SEQ ID NO: 267)). Examples of anti-RAN antibodies targeting the C-terminus of RAN protein are disclosed, for example, in U.S. Publication No. 2013/0115603, the entire content of which is incorporated herein by reference. In some embodiments, a set (or combination) of anti-RAN antibodies (e.g., a combination of two or more anti-RAN antibodies selected from poly(CP), poly(GP), poly(G), poly(A), poly(GA), poly(GD), poly(GE), poly(GQ), poly(GR), poly(GT), poly(L), poly(LP), poly(LPAC) (SEQ ID NO: 260), poly(LS), poly(P), poly(PA), poly(PR), poly(QAGR) (SEQ ID NO: 261), poly(RE), polySer, poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and poly(GSKHREAE) (SEQ ID NO: 267) etc.) are administered to a subject for the purpose of treating a disease associated with RAN proteins.

An anti-RAN antibody can be a polyclonal antibody or a monoclonal antibody. Typically, polyclonal antibodies are produced by inoculation of a suitable mammal, such as a mouse, rabbit or goat. Larger mammals are often preferred as the amount of serum that can be collected is greater. An antigen is injected into the mammal. This induces the B-lymphocytes to produce IgG immunoglobulins specific for the antigen. This polyclonal IgG is purified from the mammal's serum. Monoclonal antibodies are generally produced by a single cell line (e.g., a hybridoma cell line). In some embodiments, an anti-RAN antibody is purified (e.g., isolated from serum).

A therapeutic molecule may be an antisense oligonucleotide (ASO). In general, antisense oligonucleotides block the translation of a target protein by hybridizing to an mRNA sequence encoding the target protein, thereby inhibiting protein synthesis by ribosomal machinery. In some embodiments, the antisense oligonucleotide (ASO) targets a gene comprising a microsatellite repeat sequence. In some embodiments, the antisense oligonucleotide inhibits translation of one or more RAN proteins. One skilled in the art would understand how to construct an anti-sense oligonucleotide comprising a short (approximately 15 to 30 nucleotides) with a base sequence complementary to the RAN mRNA. One skilled in the art will understand that complementarity to the RAN mRNA can be established using canonical nucleotides comprising ribose, phosphate and one of the bases adenine, guanine, cytosine, and uracil linked with the phosphodiester linkages typifying naturally occurring nucleic acids OR some of the nucleotides could be modified by replacing the ribose with an alternate saccharide moiety such as 2′-deoxyribose, or 2′-O-(2-methoxyethyl)ribose, AND/OR some or all of the nucleotides could be modified by methylation, AND/OR some or all of the phosphodiester bonds between the nucleotides could be replaced with phosphorothioate linkages. Those skilled in the art will understand that modifications of several nucleotides at both the 3′ and 5′ ends of the antisense oligonucleotide to inhibit degradation by ubiquitous terminally active RNA nucleases will improve the stability and thus half-life of the antisense oligo. However, those skilled in the art will appreciate that it is also desirable that at least some part of the antisense oligo will, once complexed with the RAN mRNA promote the activity of Ribonuclease H to promote the enzymatic degradation of the RAN mRNA once it is complexed with the antisense oligo.

In some embodiments, the therapeutic agent is an inhibitory nucleic acid. In some embodiments, the inhibitory nucleic acid is an interfering RNA selected from the group consisting of dsRNA, siRNA, shRNA, miRNA, and ami-RNA. In some embodiments, the inhibitory nucleic acid is a nucleic acid aptamer (e.g., an RNA aptamer or DNA aptamer). Generally, an inhibitory RNA molecule can be unmodified or modified. In some embodiments, an inhibitory RNA molecule comprises one or more modified oligonucleotides, e.g., phosphorothioate-, 2′-O-methyl-, etc.-modified oligonucleotides, as such modifications have been recognized in the art as improving the stability of oligonucleotides in vivo.

In some embodiments, a therapeutic agent is an effective amount of a eukaryotic initiation factor 2 (eIF2) inhibiting agent or a Protein Kinase R (PKR) inhibiting agent (e.g., an inhibitor of eIF2 and/or PKR). In some embodiments, an inhibitor of eIF2 is an inhibitor of a serine/threonine kinase. Examples of serine/threonine kinases include but are not limited to protein kinase A (PKA), protein kinase C (PKC), Mos/Raf kinases, mitogen-activated protein kinases (MAPKs), protein kinase B (AKT kinase), etc. In some embodiments, an eIF2 inhibitor is a protein kinase R (PKR) inhibitor. Inhibitors of eIF2 and PKR are described, for example in International Application Publication No. WO 2018/195110, the entire content of which is incorporated herein by reference.

In some embodiments, the therapeutic agent is a protein kinase R (PKR) variant that functions in a dominant negative manner to inhibit phosphorylation of eIF2a. As used herein, “protein kinase R (PKR) variant” refers to a protein comprising an amino acid sequence that is at least 70%, at least 80%, at least 90%, at least 95%, at least 96%, at least 97%, at least 98%, or at least 99% identical to a wild-type protein kinase R (PKR) (e.g., GenBank Accession No. NP_002750.1), wherein the variant protein comprises at least one amino acid variation (also referred to sometimes as “mutation”) relative to the amino acid sequence of the wild-type PKR.

In some embodiments, the amino acid sequence of a PKR variant is at least 75%, at least 85%, at least 95%, at least 96%, at least 97%, at least 98%, at least 99%, at least 99.5%, at least 99.6%, at least 99.7%, at least 99.8%, or at least 99.9% identical to the amino acid sequence of wild-type PKR. In some embodiments, the amino acid sequence is about 95-99.9% identical to the amino acid sequence of wild-type PKR. In some embodiments, the protein comprises at least 1, at least 2, at least 3, at least 4, at least 5, at least 6, at least 7, at least 8, at least 9, at least 10, at least 11, at least 12, at least 13, at least 14, or at least 15 different amino acid sequence variations as compared to the sequence of amino acids set forth in the amino acid sequence of wild-type PKR. In some embodiments, a PKR variant comprises a mutation at position 296 (e.g., position 296 of a human wild-type PKR). In some embodiments, the mutation at position 296 is K296R.

An eIF2 inhibitor may be a direct inhibitor or an indirect inhibitor. Generally, a direct modulator functions by interacting with (e.g., interacting with or binding to) a gene encoding eIF2 (or eIF2α), or an eIF2 protein complex. Generally, an indirect modulator functions by interacting with a gene or protein that regulates the expression or activity of eIF2 or an eIF2α (e.g., does not directly interact with a gene or protein encoding eIF2 or an eiF2α).

In some embodiments, an inhibitor eIF2 or PKR is a selective inhibitor. A “selective inhibitor” refers to an inhibitor of eIF2 or PKR that preferentially inhibits activity or expression of one type of eIF2 subunit compared with other types of eIF2 subunits, or inhibits activity or expression of PKR preferentially compared to other kinases. In some embodiments, an inhibitor of eIF2 is a selective inhibitor of eIF2a. In some embodiments, an inhibitor of eIF2 is a selective inhibitor of eIF2A. In some embodiments, an inhibitor of eIF2 is a selective inhibitor of protein kinase R (PKR), such as a selective PKR inhibitor.

Examples of proteins that inhibit eiF2 (e.g., an eIF2 subunit) include but are not limited to polyclonal anti-eIF2 antibodies, monoclonal anti-eIF2 antibodies, etc. Examples of nucleic acid molecules that inhibit eiF2 (e.g., an eIF2 subunit) include but are not limited to dsRNA, siRNA, miRNA, etc. that target a gene encoding an eIF2 subunit (e.g., a gene encoding the mRNA set forth in GenBank Accession No. NM_004094.4). Examples of small molecule inhibitors of eIF2 include but are not limited to LY 364947, eIF-2a Inhibitor II Sal003, etc.

Examples of proteins that inhibit PKR include but are not limited to certain dominant negative PKR variants (e.g., K296R PKR mutant), TARBP2, etc. Examples of nucleic acid molecules that inhibit PKR include but are not limited to dsRNA, siRNA, miRNA, etc. that target a gene encoding a PKR. Examples of small molecule inhibitors of PKR include but are not limited to 6-amino-3-methyl-2-oxo-N-phenyl-2,3-dihydro-1H-benzo[d]imidazole-1-carboxamide, N-[2-(1H-indol-3-yl)ethyl]-4-(2-methyl-1H-indol-3-yl)pyrimidin-2-amine, metformin, buformin, phenformin, etc.

Examples of nucleic acid molecules that inhibit eIF2A include but are not limited to dsRNA, siRNA, miRNA, etc. that target a gene encoding a eIF2A (e.g., a gene encoding the mRNA set forth in GenBank Accession No. NM_032025.4). Examples of small molecule inhibitors of eIF2A include but are not limited to salubrinal, Sa1003, ISRIB, etc.

In some embodiments, the eIF2 inhibitor or PKR inhibitor is an interfering (e.g., inhibitory) nucleic acid. In some embodiments, the inhibitory nucleic acid is an interfering RNA selected from the group consisting of dsRNA, siRNA, shRNA, mi-RNA, and ami-RNA. In some embodiments, the inhibitory nucleic acid is an antisense nucleic acid (e.g., an antisense oligonucleotide (ASO) or a nucleic acid aptamer (e.g., an RNA aptamer). Generally, an inhibitory RNA molecule can be unmodified or modified. In some embodiments, an inhibitory RNA molecule comprises one or more modified oligonucleotides, e.g., phosphorothioate-, 2′-O-methyl-, etc.-modified oligonucleotides, as such modifications have been recognized in the art as improving the stability of oligonucleotides in vivo.

In some embodiments, the interfering RNA comprises a sequence that is complementary with between 5 and 50 continuous nucleotides (e.g., 5, 6, 7, 8, 9, 10, 11, 12, 13, 14, 15, 16, 17, 18, 19, 20, 21, 22, 23, 24, 25, about 30, about 35, about 40, or about 50 continuous nucleotides) of a nucleic acid sequence (such as an RNA sequence) encoding an eIF2 subunit or a nucleic acid sequence (such as an RNA sequence) encoding PKR.

In some embodiments, a therapeutic agent is an inhibitor of Eukaryotic initiation factor 3 (eIF3), which is a multiprotein complex that is involved with the initiation phase of eukaryotic protein translation. Generally, in humans eIF3 comprises 13 non-identical subunits (e.g., eIF3a-m). Mammalian eIF3, the largest most complex initiation factor, comprises up to 13 non-identical subunits. Typically, eIF3f is involved in many steps of translation initiation including stabilization of the ternary complex, mediating binding of mRNA to 40S subunit and facilitating dissociation of 40S and 60S ribosomal subunits. In some embodiments, therapeutic agents that inhibit expression or activity of an eIF3 subunit (e.g., eIF3f, eIF3m, eIF3h, or other eIF3 subunit) can be used to reduce or inhibit RAN translation in a cell or in a subject (e.g., a subject having Alzheimer's disease characterized by RAN protein translation). Inhibitors of eIF3 subunits are further described, for example in International Application Publication No. WO 2017/176813, the entire content of which is incorporated herein by reference.

An eIF3 inhibitor may be a direct inhibitor or an indirect inhibitor. Generally, a direct modulator functions by interacting with (e.g., interacting with or binding to) a gene encoding eIF3 (or an eIF3 subunit), or an eIF3 protein complex, or an eIF3 subunit. Generally, an indirect modulator functions by interacting with a gene or protein that regulates the expression or activity of eIF3 or an eIF3 subunit (e.g., does not directly interact with a gene or protein encoding eIF3 or an eiF3 subunit). In some embodiments, an inhibitor of eIF3 is a selective inhibitor. A “selective inhibitor” refers to a modulator of eIF3 that preferentially inhibits activity or expression of one type of eIF3 subunit compared with other types of eIF3 subunits. In some embodiments, an inhibitor of eIF3 is a selective inhibitor of eIF3f.

An eIF3 inhibitor can be a protein (e.g., antibody), nucleic acid, or small molecule. Examples of proteins that inhibit eiF3 (e.g., an eIF3 subunit) include but are not limited to polyclonal anti-eIF3 antibodies, monoclonal anti-eIF3 antibodies, Measles Virus N protein, Viral stress-inducible protein p56, etc. Examples of nucleic acid molecules that inhibit eiF3 (e.g., an eIF3 subunit) include but are not limited to dsRNA, siRNA, miRNA, amiRNA, etc. that target a gene encoding an eIF3 subunit. Examples of small molecule inhibitors of eIF3 include but are not limited to mTOR inhibitors (e.g., rapamycin, PP242), S6 kinase (S6K) inhibitors, etc.

In some embodiments, an interfering RNA comprises a sequence that is complementary with between 5 and 50 continuous nucleotides (e.g., 5, 6, 7, 8, 9, 10, 11, 12, 13, 14, 15, 16, 17, 18, 19, 20, 21, 22, 23, 24, 25, about 30, about 35, about 40, or about 50 continuous nucleotides) of a nucleic acid sequence (such as an RNA sequence) encoding an eIF3 subunit. Examples of nucleic acid sequences encoding eIF3 subunits include GenBank Accession No. NM_003750.2 (eIF3a), GenBank Accession No. NM_003751.3 (eIF3b), GenBank Accession No. NM_003752.4 (eIF3c), GenBank Accession No. NM_003753.3 (eIF3d), GenBank Accession No. NM_001568.2 (eIF3e), GenBank Accession No. NM_003754.2 (eIF3f), GenBank Accession No. NM_003755.4 (eIF3g), GenBank Accession No. NM_003756.2 (eIF3h), GenBank Accession No. NM_003757.3 (eIF3i), GenBank Accession No. NM_003758.3 (eIF3j), GenBank Accession No. NM_013234.3 (eIF3k), GenBank Accession No. NM_016091.3 (eIF31), GenBank Accession No. NM_006360.5 (eiF3m), etc. In some embodiments, the interfering RNA is a siRNA. In some embodiments, an eIF3f siRNA is administered (e.g., Dharmacon Cat #J-019535-08). In some embodiments, an eIF3m siRNA is administered (e.g., Dharmacon Cat #J-016219-12). In some embodiments, an eIF3h siRNA is administered (e.g., Dharmacon Cat #J-003883-07).

In some embodiments, eIF3f is a negative regulator of RAN translation and decreased levels of human eIF3f are associated with decreased accumulation of RAN protein in cells. In some embodiments, RAN translation (e.g., in cells expressing a RAN protein) is sensitive to eIF3f knockdown unlike translation from close cognate or AUG translation. In some embodiments, the translational machinery used for RAN translation is distinct from AUG and near AUG translation machinery in a cell.

In some embodiments, a therapeutic agent is an inhibitor of TLR3. An inhibitor of TLR3 can be a protein (e.g., antibody), nucleic acid, or small molecule. Examples of proteins that inhibit TLR3 include but are not limited to polyclonal anti-TLR3 antibodies, monoclonal anti-TLR3 antibodies, etc. Examples of nucleic acid molecules that inhibit TLR3 include but are not limited to dsRNA, siRNA, miRNA, amiRNA, etc. that target a gene encoding TLR3. Examples of small molecule inhibitors of TLR3 are described, for example in Cheng et al. (2011) J Am Chem Soc 133(11):3764-7.

In some embodiments, a therapeutic agent is an inhibitor of p62 protease. An inhibitor of p62 can be a protein (e.g., antibody), nucleic acid, or small molecule. Examples of proteins that inhibit p62 include but are not limited to polyclonal anti-p62 antibodies, monoclonal anti-p62 antibodies, etc. Examples of nucleic acid molecules that inhibit p62 include but are not limited to dsRNA, siRNA, miRNA, amiRNA, etc. that target a gene encoding p62. In some embodiments, a therapeutic agent is an agent that increases proteasome activity, for example as described in Leestemaker et al. (2017) Cell Chemical Biology 24, 725-736.

In some embodiments, a therapeutic agent comprises a peptide antigen that targets one or more RAN proteins (e.g., is a RAN protein vaccine that targets one or more RAN proteins). In some embodiments, the peptide antigen targets (e.g., comprises an amino acid sequence encoding) one or more of the RAN proteins poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and poly(GSKHREAE) (SEQ ID NO: 267).

In some embodiments, one or more therapeutic molecules are administered to a subject to treat a disease associated with RAN proteins characterized by an expansion of a nucleic acid repeat (e.g., associated with a repeat associated non-ATG translation). For example, in some embodiments, a subject is administered 2, 3, 4, 5, 6, 7, 8, 9, or 10 therapeutic agents (e.g., proteins, nucleic acids, small molecules, etc., or any combination thereof).

### Monoclonal Antibodies

Various aspects of the disclosure relate to antibodies and antigen-binding fragments that specifically bind to RAN proteins, and methods of making and using the same. In some embodiments, the antibody or antigen binding fragment specifically binds one or more of poly(glycine-alanine) [poly(GA)], poly(proline-arginine) [poly(PR)], poly(glycine-arginine) [poly(GR)], poly-Serine (polySer), poly(glycine-proline) [poly(GP)], poly-Leucine (polyLeu), poly-Alanine (polyAla), poly(leucine-proline-alanine-cysteine) [poly(LPAC)] (SEQ ID NO: 260), and poly(glutamine-alanine-glycine-arginine) [poly(QAGR)] (SEQ ID NO: 261). In some embodiments, the antibody or antigen-binding fragment specifically binds poly(GA). In some embodiments, the antibody or antigen-binding fragment specifically binds polySer. In some embodiments, the antibody or antigen-binding fragment specifically binds poly(PR). In some embodiments, the antibody or antigen-binding fragment specifically binds poly(GR). In some embodiments, the antibody or antigen-binding fragment specifically binds polyLeu. In some embodiments, the antibody or antigen-binding fragment specifically binds polyAla. In some embodiments, the antibody or antigen-binding fragment specifically binds poly(LPAC) (SEQ ID NO: 260). In some embodiments, the antibody or antigen-binding fragment specifically binds poly(QAGR) (SEQ ID NO: 261). In some embodiments, the antibody is a monoclonal antibody. In some embodiments, the antibody or antigen-binding fragment specifically binds poly(CP). In some embodiments, the antibody or antigen-binding fragment specifically binds poly(GP). In some embodiments, the antibody or antigen-binding fragment specifically binds poly(G). In some embodiments, the antibody or antigen-binding fragment specifically binds poly(GD). In some embodiments, the antibody or antigen-binding fragment specifically binds poly(GE). In some embodiments, the antibody or antigen-binding fragment specifically binds poly(GQ). In some embodiments, the antibody or antigen-binding fragment specifically binds poly(GT). In some embodiments, the antibody or antigen-binding fragment specifically binds poly(LP). In some embodiments, the antibody or antigen-binding fragment specifically binds poly(LS). In some embodiments, the antibody or antigen-binding fragment specifically binds poly(P). In some embodiments, the antibody or antigen-binding fragment specifically binds poly(PA). In some embodiments, the antibody or antigen-binding fragment specifically binds poly(RE). In some embodiments, the antibody or antigen-binding fragment specifically binds poly(SP). In some embodiments, the antibody or antigen-binding fragment specifically binds poly(VP). In some embodiments, the antibody or antigen-binding fragment specifically binds poly(FP). In some embodiments, the antibody or antigen-binding fragment specifically binds poly(GK). In some embodiments, the antibody or antigen-binding fragment specifically binds poly(FTPLSLPV) (SEQ ID NO: 262). In some embodiments, the antibody or antigen-binding fragment specifically binds poly(LLPSPSRC) (SEQ ID NO: 263). In some embodiments, the antibody or antigen-binding fragment specifically binds poly(YSPLPPGV) (SEQ ID NO: 264). In some embodiments, the antibody or antigen-binding fragment specifically binds poly(HREGEGSK) (SEQ ID NO: 255). In some embodiments, the antibody or antigen-binding fragment specifically binds poly(TGRERGVN) (SEQ ID NO: 265). In some embodiments, the antibody or antigen-binding fragment specifically binds poly(PGGRGE) (SEQ ID NO: 258). In some embodiments, the antibody or antigen-binding fragment specifically binds poly(GRQRGVNT) (SEQ ID NO: 266). In some embodiments, the antibody or antigen-binding fragment specifically binds poly(GSKHREAE) (SEQ ID NO: 267).

An antibody, as used herein, broadly refers to an immunoglobulin molecule or any functional mutant, variant, or derivation thereof. It is desired that functional mutants, variants, and derivations thereof, as well as antigen-binding fragments, retain the essential epitope binding features of an Ig molecule. Antibodies are capable of specific binding to a target through at least one antigen recognition site, located in the variable region of the immunoglobulin molecule. Generally, an intact or full-length antibody comprises two heavy chains and two light chains. Each heavy chain contains a heavy chain variable region (VH) and a first, second and third constant regions (CH1, CH2 and CH3). Each light chain contains a light chain variable region (VL) and a constant region (CL). The VH and VL regions can be further subdivided into regions of hypervariability, termed complementarity determining regions (CDR), interspersed with regions that are more conserved, termed framework regions (FR). CDR constituents on the heavy chain are referred to as CDRH1, CDRH2, and CDRH3, while CDR constituents on the light chain are referred to as CDRL1, CDRL2, and CDRL3.

The CDRs typically refer to the Kabat CDRs, as described in Sequences of Proteins of Immunological Interest, US Department of Health and Human Services (1991), eds. Kabat et al. Another standard for characterizing the antigen binding site is to refer to the hypervariable loops as described by Chothia. See, e.g., Chothia, D. et al. (1992) J. Mol. Biol. 227:799-817; and Tomlinson et al. (1995) EMBO J. 14:4628-4638. Still another standard is the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg). Embodiments described with respect to Kabat CDRs can alternatively be implemented using similar described relationships with respect to Chothia hypervariable loops or to the AbM-defined loops, or combinations of any of these methods.

Each VH and VL is composed of three CDRs and four FRs, arranged from amino-terminus to carboxy-terminus in the following order: FR1, CDR1, FR2, CDR2, FR3, CDR3, FR4. A full-length antibody can be an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The term “antigen-binding fragment” refers to any derivative of an antibody which is less than full-length, and that can bind specifically to a target. Preferably, antigen-binding fragments provided herein retain the ability to specifically bind to RAN protein. An antigen-binding fragment may comprise the heavy chain variable region (VH), the light chain variable region (VL), or both. Each of the VH and VL typically contains three complementarity determining regions CDR1, CDR2, and CDR3.

Examples of antigen binding fragments include, but are not limited to, Fab, Fab′, F(ab′)2, scFv, Fv, dsFv, diabody, affibodies, and Fd fragments. Antigen binding fragments may be produced by any appropriate means. For instance, an antigen binding fragment may be enzymatically or chemically produced by fragmentation of an intact antibody or it may be recombinantly produced from a gene encoding the partial antibody sequence. Alternatively, an antigen binding fragment may be wholly or partially synthetically produced. An antigen binding fragment may optionally be a single chain antibody fragment. Alternatively, a fragment may comprise multiple chains which are linked together, for instance, by disulfide linkages. An antigen binding fragment may also optionally be a multimolecular complex. A functional antigen binding fragment will typically comprise at least about 50 amino acids and more typically will comprise at least about 200 amino acids.

Single-chain Fvs (scFvs) are recombinant antigen binding fragments consisting of only the variable light chain (VL) and variable heavy chain (VH) covalently connected to one another by a polypeptide linker. Either VL or VH may be the NH2-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility. ScFvs are encompassed within the term “antigen-binding fragment.”

Diabodies are dimeric scFvs. The components of diabodies typically have shorter peptide linkers than most scFvs, and they show a preference for associating as dimers (see, e.g., Holliger, P., et al. (1993) Proc. Natl. Acad. Sci. USA 90:6444-6448; Poljak, R. J., et al. (1994) Structure 2: 1121-1123). Diabodies are also encompassed within the term “antigen-binding fragment.”

A Fv fragment is an antigen binding fragment which consists of one VH and one VL domain held together by noncovalent interactions. Although the two domains of the Fv fragment, VL and VH, can be coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. The term dsFv is used herein to refer to an Fv with an engineered intermolecular disulfide bond to stabilize the VH-VL pair. dsFvs are also encompassed within the term “antigen-binding fragment.”

A F(ab′)2 fragment is an antigen binding fragment essentially equivalent to that obtained from immunoglobulins (typically IgG) by digestion with an enzyme pepsin at pH 4.0-4.5. The fragment may be recombinantly produced. F(ab′)2 are also encompassed within the term “antigen-binding fragment.”

A Fab fragment is an antigen binding fragment essentially equivalent to that obtained by reduction of the disulfide bridge or bridges joining the two heavy chain pieces in the F(ab′)2 fragment. The Fab′ fragment may be recombinantly produced. Fab′ are also encompassed within the term “antigen-binding fragment.”

A Fab fragment is an antigen binding fragment essentially equivalent to that obtained by digestion of immunoglobulins (typically IgG) with the enzyme papain. The Fab fragment may be recombinantly produced. The heavy chain segment of the Fab fragment is the Fd piece. Fab fragments are also encompassed within the term “antigen-binding fragment.”

An affibody is a small protein comprising a three-helix bundle that functions as an antigen binding molecule (e.g., an antibody mimetic). Generally, affibodies are approximately 58 amino acids in length and have a molar mass of approximately 6 kDa. Affibody molecules with unique binding properties are acquired by randomization of 13 amino acids located in two alpha-helices involved in the binding activity of the parent protein domain. Specific affibody molecules binding a desired target protein can be isolated from pools (libraries) containing billions of different variants, using methods such as phage display. Affibodies are also encompassed within the term “antigen-binding fragment.”

The term “human antibody” refers to antibodies having variable and constant regions corresponding substantially to, or derived from, antibodies obtained from human subjects, e.g., encoded by human germline immunoglobulin sequences or variants thereof. Human antibodies may include one or more amino acid residues not encoded by human germline immunoglobulin sequences (e.g., mutations introduced by random or site-specific mutagenesis in vitro or by somatic mutation in vivo). Such mutations may present in one or more of the CDRs, particularly CDR3, or in one or more of the framework regions. In some embodiments, the human antibodies may have at least one, two, three, four, five, or more positions replaced with an amino acid residue that is not encoded by the human germline immunoglobulin sequence. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29: 128-145; Hoogenboom H., and Chames P. (2000) Immunology Today 21:371-378), antibodies isolated from an animal (e.g., a mouse) that is transgenic for human immunoglobulin genes (see, e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions as defined above. In certain embodiments, however, such recombinant human antibodies may be subjected to in vitro mutagenesis (or, when an animal transgenic for human Ig sequences is used, in vivo somatic mutagenesis) and thus the amino acid sequences of the VH and VL regions of the recombinant antibodies may be sequences that, while derived from and related to human germline VH and VL sequences, may not naturally exist within the human antibody germline repertoire in vivo.

In some embodiments, the antibody or antigen binding fragment specifically binds an amino acid sequence as set forth in any one or more of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, and 56.

In some embodiments, the antibody or antigen-binding fragment comprises a heavy chain constant region comprising an amino acid sequence represented by SEQ ID NOs: 195. In some embodiments, the antibody or antigen-binding fragment comprises a heavy chain constant region comprising a nucleic acid sequence represented by SEQ ID NOs: 197. In some embodiments, the anti-RAN antibodies and antigen binding fragments of the disclosure comprise a light chain constant region comprising an amino acid sequence represented by SEQ ID NOs: 196. In some embodiments, the anti-RAN antibodies and antigen binding fragments of the disclosure comprise a light chain constant region comprising a nucleic acid sequence represented by SEQ ID NO: 198.

In some embodiments, the anti-RAN antibodies or antigen binding fragments may or may not include the framework region of the antibodies, for example the framework region amino acid sequences as set forth in SEQ ID NOs: 155-186. In some embodiments, anti-RAN antibodies are murine antibodies. In some embodiments, anti-RAN antibodies are chimeric or humanized antibodies.

In some embodiments, the antibody or antigen binding fragment comprises a VH sequence as set forth in SEQ ID NO. 109, 111, 113 or 115. In some embodiments, the antibody or antigen binding fragment comprises a VL sequence as set forth in SEQ ID NO. 110, 112, 114 or 116.

In some embodiments, the antibody or antigen binding fragment comprises a VH sequence as set forth in SEQ ID NO. 109 and a VL sequence as set forth in SEQ ID NO. 110.

In some embodiments, the antibody or antigen binding fragment comprises a VH sequence as set forth in SEQ ID NO. 111 and a VL sequence as set forth in SEQ ID NO. 112.

In some embodiments, the antibody or antigen binding fragment comprises a VH sequence as set forth in SEQ ID NO. 113 and a VL sequence as set forth in SEQ ID NO. 114.

In some embodiments, the antibody or antigen binding fragment comprises a VH sequence as set forth in SEQ ID NO. 115 and a VL sequence as set forth in SEQ ID NO. 116.

In some embodiments, antibody or antigen-binding fragment comprises six complementarity determining regions (CDRs): CDRH1, CDRH2, CDRH3, CDRL1, CDRL2, and CDRL3, wherein CDRH1 comprises a sequence as set forth in SEQ ID NO: 117, CDRH2 comprises a sequence as set forth in SEQ ID NO: 125, CDRH3 comprises a sequence as set forth in SEQ ID NO: 133, CDRL1 comprises a sequence as set forth in SEQ ID NO: 118, CDRL2 comprises a sequence as set forth in SEQ ID NO: 126, and CDRL3 comprises a sequence as set forth in SEQ ID NO: 134.

In some embodiments, antibody or antigen-binding fragment comprises six complementarity determining regions (CDRs): CDRH1, CDRH2, CDRH3, CDRL1, CDRL2, and CDRL3, wherein CDRH1 comprises a sequence as set forth in SEQ ID NO: 119, CDRH2 comprises a sequence as set forth in SEQ ID NO: 127, CDRH3 comprises a sequence as set forth in SEQ ID NO: 135, CDRL1 comprises a sequence as set forth in SEQ ID NO: 120, CDRL2 comprises a sequence as set forth in SEQ ID NO: 128, and CDRL3 comprises a sequence as set forth in SEQ ID NO: 136.

In some embodiments, antibody or antigen-binding fragment comprises six complementarity determining regions (CDRs): CDRH1, CDRH2, CDRH3, CDRL1, CDRL2, and CDRL3, wherein CDRH1 comprises a sequence as set forth in SEQ ID NO: 121, CDRH2 comprises a sequence as set forth in SEQ ID NO: 129, CDRH3 comprises a sequence as set forth in SEQ ID NO: 137, CDRL1 comprises a sequence as set forth in SEQ ID NO: 122, CDRL2 comprises a sequence as set forth in SEQ ID NO: 130, and CDRL3 comprises a sequence as set forth in SEQ ID NO: 138.

In some embodiments, antibody or antigen-binding fragment comprises six complementarity determining regions (CDRs): CDRH1, CDRH2, CDRH3, CDRL1, CDRL2, and CDRL3, wherein CDRH1 comprises a sequence as set forth in SEQ ID NO: 123, CDRH2 comprises a sequence as set forth in SEQ ID NO: 131, CDRH3 comprises a sequence as set forth in SEQ ID NO: 139, CDRL1 comprises a sequence as set forth in SEQ ID NO: 124, CDRL2 comprises a sequence as set forth in SEQ ID NO: 132, and CDRL3 comprises a sequence as set forth in SEQ ID NO: 140.

It should be appreciated that, in some embodiments, the disclosure contemplates variants (e.g., homologs) of amino acid and nucleic acid sequences for the heavy chain variable region and light chain variable region of the antibodies. “Homology” refers to the percent identity between two polynucleotides or two polypeptide moieties. The term “substantial homology”, when referring to a nucleic acid, or fragment thereof, indicates that, when optimally aligned with appropriate nucleotide insertions or deletions with another nucleic acid (or its complementary strand), there is nucleotide sequence identity in about 90 to 100% of the aligned sequences. For example, in some embodiments, nucleic acid sequences sharing substantial homology are at least 90%, at least 91%, at least 92% at least 93%, at least 94%, at least 95%, at least 96% at least 97%, at least 98% at least 99% sequence identity. When referring to a polypeptide, or fragment thereof, the term “substantial homology” indicates that, when optimally aligned with appropriate gaps, insertions or deletions with another polypeptide, there is nucleotide sequence identity in about 90 to 100% of the aligned sequences. The term “highly conserved” means at least 80% identity, preferably at least 90% identity, and more preferably, over 97% identity. For example, in some embodiments, highly conserved proteins share at least 85%, at least 90%, at least 91%, at least 92% at least 93%, at least 94%, at least 95%, at least 96% at least 97%, at least 98% at least 99% identity. In some cases, highly conserved may refer to 100% identity. Identity is readily determined by one of skill in the art by, for example, the use of algorithms and computer programs known by those of skill in the art.

In some embodiments, RAN antibodies of the disclosure can bind to a RAN protein with high affinity, e.g., with a Kd less than 10⁻⁷ M, 10⁻⁸M, 10⁻⁹M, 10⁻¹⁰ M, 10⁻¹¹ M or lower. For example, anti-RAN antibodies or antigen binding fragments can bind to a RAN protein with an affinity between 5 pM and 500 nM, e.g., between 50 pM and 100 nM, e.g., between 500 pM and 50 nM. The disclosure also includes antibodies or antigen binding fragments that compete with any of the antibodies described herein for binding to RAN proteins and that have an affinity of 50 nM or lower (e.g., 20 nM or lower, 10 nM or lower, 500 pM or lower, 50 pM or lower, or 5 pM or lower). The affinity and binding kinetics of the anti-RAN protein antibody can be tested using any method known in the art including but not limited to biosensor technology (e.g., OCTET or BIACORE).

In some embodiments, anti-RAN antibodies of the present disclosure include the VH, VL, and CDR, amino acid sequences shown in Table 4 below.

TABLE 4
Amino Acid Sequences of Anti-RAN Antibodies

| Clone | region | Variable Region | CDR1 | CDR2 | CDR3 |
|---|---|---|---|---|---|
| 27B11.A7 | VH | EVQLQESGGGSVQPGGSLK | GFAFSNYG | INTSDGDST | ARVGGNY |
| | | LSCAASGFAFSNYGMSWVR | (SEQ ID NO: | (SEQ ID | DFAMDY |
| | | QTPDKRLELVTTINSDGDST | 117) | NO: 125) | (SEQ ID |
| | | FYPDSVKGRFTISRDNAKN | | | NO: 133) |
| | | ALYLQMSSLKSDDTAMYY | | | |
| | | CARVGGNYDFAMDYWGQ | | | |
| | | GTSVIVSS (SEQ ID NO: 109) | | | |
| 27B11.A7 | VL | DIVMSQFPSSLAVSAGDKV | QSLLNSRTR | WTS (SEQ | KQSYNNP |
| | | TMSCKSSQSLLNSRTRKNY | KNY (SEQ | ID NO: | WT (SEQ ID |
| | | LAWYQQKPGQSPKLLIYWT | ID NO: 118) | 126) | NO: 134) |
| | | STRESGVPDRFTGSRSGTDF | | | |
| | | TLTISSVQAEDLAVYYCKQS | | | |
| | | YNNPWTFGGGTKLEIK | | | |
| | | (SEQ ID NO: 110) | | | |
| 23H2.D1.B5 | VH | EVQLQESGGGSVQPGGALQ | GFTFSSHG | INTSNGGST | ARVGDND |
| | | LSCAASGFTFSSHGMSWVR | (SEQ ID NO: | (SEQ ID | DFAMGY |
| | | QTPDKRLEMVATINSNGGS | 119) | NO: 127) | (SEQ ID |
| | | TYYPDSVKGRFIISRDNAKN | | | NO: 135) |
| | | TLYLQMSSLKSEDTAMYYC | | | |
| | | ARVGDNDDFAMGYWGQG | | | |
| | | TSVTVSS (SEQ ID NO: 111) | | | |
| 23H2.D1.B5 | VL | DIVMSQSPSSLAVSEGEKVT | QSLFNSRTR | WTS (SEQ | KQSYNNP |
| | | LTCKSSQSLFNSRTRKNYLA | KNY (SEQ | ID NO: | WT (SEQ ID |
| | | WYQQKPGQPPKLLIYWTST | ID NO: 120) | 126) | NO: 134) |
| | | RESGVPDRFTGSGYGTDFTL | | | |
| | | TISSVQAEDLAVYYCKQSY | | | |
| | | NNPWTFGGGTKLEIK (SEQ | | | |
| | | ID NO: 112) | | | |
| 16A3.C8 | VH | EVQLQESGSEVVRPGASVK | GYTFTSYW | VYPGSGL | TRSAYSW |
| | | LSCKASGYTFTSYWLHWV | (SEQ ID NO: | T (SEQ ID | YDYGMDC |
| | | KQRPGQGLEWIGNVYPGSG | 121) | NO: 129) | (SEQ ID |
| | | LTGYDEKFRTKATVTVDTS | | | NO: 137) |
| | | SSTAYMQLSSLTTEDSAVY | | | |
| | | YCTRSAYSWYDYGMDCW | | | |
| | | GQGTSVTVST (SEQ ID NO: | | | |
| | | 113) | | | |
| 16A3.C8 | VL | QIVLTQSPEILSASPGEKVT | SSVNY (SEQ | DTS (SEQ | HQWSSNPP |
| | | MTCNATSSVNYMHWYQQK | ID NO: 122) | ID NO: | T (SEQ ID |
| | | SGTSPKRWIYDTSKLASGVP | | 130) | NO: 138) |
| | | ARFSGSGSGTSYSLTISSME | | | |
| | | AEDAAAYYCHQWSSNPPTF | | | |
| | | GSGTKLEIK (SEQ ID NO: | | | |
| | | 114) | | | |
| HL23 | VH | EVQLQQSGAELVRSGASVK | DFYIQ (SEQ | WIDPENG | GDYDSHY |
| 62- | | LSCTASGFNIRDFYIQWVKQ | ID NO: 123) | DTEYAPK | YSMDY |
| 2G4 | | RPEQGLEWIGWIDPENGDT | | FQG (SEQ | (SEQ ID |
| | | EYAPKFQGKATMTADTSSN | | ID NO: | NO: 139) |
| | | TAYLQLSSLTSEDTAVYYC | | 131) | |
| | | NAGDYDSHYYSMDYWGQ | GACTTCTA | TGGATTG | GGGGACT |
| | | GTSVTVSS (SEQ ID NO: 115) | TATTCAG | ATCCTGA | ATGATTCC |
| | | | (SEQ ID NO: | GAATGGT | CATTACTA |
| | | | 141) | GATACTG | TTCTATGG |
| | | | | AATATGC | ACTAC |
| | | | | CCCGAAA | (SEQ ID |
| | | | | TTCCAGG | NO: 145) |
| | | | | GC (SEQ | |
| | | | | ID NO: 143) | |
| HL23 | VL | DVLMTQTPLSLPVSLGDQA | RSSRSIVHS | KVSNRFS | YQVSHVP |
| 62- | | SISCRSSRSIVHSNGNTYLE | NGNTYLE | (SEQ ID | WT (SEQ ID |
| 2G4 | | WYLQKPGQSPKLLIYKVSN | (SEQ ID NO: | NO: 132) | NO: 140) |
| | | RFSGVPDRFSGSGSGTDFTL | 124) | | |
| | | KISRVEADDLGVYYCYQVS | AGATCTAG | AAAGTTT | TATCAAGT |
| | | HVPWTFGGGTKLEIK (SEQ | TCGGAGCA | CCAACCG | TTCACATG |
| | | ID NO: 116) | TTGTACAT | ATTTTCT | TTCCGTGG |
| | | | AGTAATGG | (SEQ ID | ACG (SEQ |
| | | | AAACACCT | NO: 144) | ID NO: 146) |
| | | | ATTTAGAA | | |
| | | | (SEQ ID NO: | | |
| | | | 142) | | |

TABLE 5
Nucleic Acid Sequences of Anti-RAN Antibodies

| Clone | region | Variable Region |
|---|---|---|
| 27B11.A7 | VH | GAGGTGCAGCTGCAGGAGTCTGGGGGAGGCTCAGTGC |
| | | AGCCTGGAGGGTCCCTGAAACTCTCCTGCGCAGCCTC |
| | | TGGATTCGCTTTCAGTAACTATGGCATGTCTTGGGTTC |
| | | GCCAGACTCCAGACAAGAGGCTGGAGTTGGTCACAAC |
| | | CATTAATAGTGATGGTGATAGTACCTTTTATCCAGACA |
| | | GTGTGAAGGGCCGATTCACCATCTCCAGAGACAATGC |
| | | CAAGAACGCCCTGTACCTGCAAATGAGCAGTCTGAAG |
| | | TCAGACGACACAGCCATGTATTACTGTGCAAGAGTGG |
| | | GAGGTAACTACGACTTTGCTATGGACTACTGGGGTCA |
| | | GGGAACCTCAGTCATCGTCTCCTCAG (SEQ ID NO: 147) |
| 27B11.A7 | VL | GACATTGTGATGTCACAGTTTCCATCCTCCCTGGCTGT |
| | | GTCAGCAGGAGATAAGGTCACTATGAGCTGCAAATCC |
| | | AGTCAGAGTCTGCTCAACAGTAGGACCCGAAAGAACT |
| | | ACTTGGCTTGGTACCAGCAGAAACCAGGGCAGTCTCC |
| | | TAAACTACTGATCTACTGGACATCCACTCGGGAATCT |
| | | GGGGTCCCTGATCGCTTCACAGGCAGTCGATCTGGGA |
| | | CAGATTTCACTCTCACCATCAGCAGTGTGCAGGCTGA |
| | | AGACCTGGCAGTTTATTACTGCAAGCAATCTTATAATA |
| | | ATCCGTGGACGTTCGGTGGAGGCACCAAGCTGGAAAT |
| | | AAAAC (SEQ ID NO: 148) |
| 23H2.D1.B5 | VH | GAGGTGCAGCTGCAGGAGTCTGGGGGAGGCTCAGTGC |
| | | AGCCTGGAGGGGCCCTGCAACTCTCCTGTGCAGCCTC |
| | | TGGATTCACTTTCAGTAGTCATGGCATGTCTTGGGTTC |
| | | GCCAGACTCCAGACAAGAGGCTGGAAATGGTCGCAAC |
| | | CATTAATAGTAATGGTGGGAGTACCTATTACCCAGAC |
| | | AGTGTGAAGGGCCGATTCATCATCTCCAGAGACAATG |
| | | CCAAAAACACCCTGTACCTGCAAATGAGCAGTCTGAA |
| | | GTCTGAGGACACAGCCATGTATTACTGTGCAAGAGTG |
| | | GGAGATAACGACGACTTTGCTATGGGCTACTGGGGTC |
| | | AAGGAACCTCAGTCACCGTCTCCTCAG (SEQ ID NO: |
| | | 149) |
| 23H2.D1.B5 | VL | GACATTGTGATGTCACAGTCTCCATCCTCCCTGGCTGT |
| | | GTCAGAAGGAGAGAAGGTCACTTTAACCTGCAAATCC |
| | | AGTCAGAGTTTGTTCAACAGTAGAACCCGAAAGAACT |
| | | ACTTGGCTTGGTACCAGCAGAAACCAGGGCAGCCTCC |
| | | TAAACTGTTGATCTACTGGACATCCACTAGGGAATCT |
| | | GGGGTCCCTGATCGCTTCACAGGCAGTGGATATGGGA |
| | | CAGATTTCACTCTCACCATCAGCAGTGTGCAGGCTGA |
| | | AGACCTGGCAGTTTATTACTGCAAACAATCTTATAATA |
| | | ATCCGTGGACGTTCGGTGGAGGCACCAAGTTGGAAAT |
| | | AAAAC (SEQ ID NO: 150) |
| 16A3.C8 | VH | GAGGTGCAGCTGCAGGAGTCTGGGTCTGAGGTGGTGA |
| | | GGCCTGGAGCTTCAGTGAAGCTGTCCTGCAAGGCTTC |
| | | TGGCTACACATTCACCAGCTACTGGCTGCACTGGGTG |
| | | AAGCAGAGGCCTGGACAAGGCCTTGAGTGGATTGGAA |
| | | ATGTTTATCCTGGTAGTGGTCTTACTGGCTACGATGAA |
| | | AAATTCAGGACCAAGGCCACAGTGACTGTAGACACAT |
| | | CCTCCAGCACAGCCTACATGCAACTCAGCAGCCTGAC |
| | | AACTGAGGACTCTGCGGTCTATTACTGTACAAGATCG |
| | | GCCTACTCTTGGTACGACTATGGAATGGACTGCTGGG |
| | | GTCAAGGAACCTCAGTCACAGTCTCTACAG (SEQ ID |
| | | NO: 151) |
| 16A3.C8 | VL | CAAATTGTTCTCACCCAGTCTCCAGAAATCTTGTCTGC |
| | | ATCTCCAGGGGAGAAGGTCACCATGACCTGCAATGCC |
| | | ACCTCAAGTGTAAATTATATGCACTGGTACCAGCAGA |
| | | AGTCAGGCACCTCCCCCAAAAGATGGATTTATGACAC |
| | | ATCCAAACTGGCTTCTGGAGTCCCTGCTCGCTTCAGTG |
| | | GCAGTGGGTCTGGGACCTCTTATTCTCTCACAATCAGC |
| | | AGCATGGAGGCTGAAGATGCTGCCGCTTATTACTGCC |
| | | ACCAGTGGAGTAGTAACCCACCCACGTTCGGCTCGGG |
| | | GACAAAGCTGGAAATCAAAC (SEQ ID NO: 152) |
| HL2362- | VH | GAGGTTCAGCTGCAGCAGTCTGGGGCAGAACTTGTGA |
| 2G4 | | GGTCAGGGGCCTCAGTCAAGTTGTCCTGCACAGCTTCT |
| | | GGCTTCAACATTAGAGACTTCTATATTCAGTGGGTGA |
| | | AACAGAGGCCTGAACAGGGCCTGGAGTGGATTGGATG |
| | | GATTGATCCTGAGAATGGTGATACTGAATATGCCCCG |
| | | AAATTCCAGGGCAAGGCCACTATGACTGCAGACACAT |
| | | CCTCCAACACAGCCTACCTGCAGCTCAGCAGCCTGAC |
| | | ATCTGAGGACACTGCCGTCTATTACTGTAATGCAGGG |
| | | GACTATGATTCCCATTACTATTCTATGGACTACTGGGG |
| | | TCAAGGAACCTCTGTCACCGTCTCCTCA (SEQ ID NO: |
| | | 153) |
| HL2362- | VL | GATGTTTTGATGACCCAAACTCCACTCTCCCTGCCTGT |
| 2G4 | | CAGTCTTGGAGATCAAGCCTCCATCTCTTGCAGATCTA |
| | | GTCGGAGCATTGTACATAGTAATGGAAACACCTATTT |
| | | AGAATGGTACCTGCAGAAACCAGGCCAGTCTCCAAAG |
| | | CTCCTGATCTACAAAGTTTCCAACCGATTTTCTGGGGT |
| | | CCCAGACAGGTTCAGTGGCAGTGGATCAGGGACAGAT |
| | | TTCACACTCAAGATCAGCAGAGTGGAGGCTGATGATC |
| | | TGGGAGTTTATTACTGCTATCAAGTTTCACATGTTCCG |
| | | TGGACGTTCGGTGGAGGCACCAAGCTGGAAATCAAA |
| | | (SEQ ID NO: 154) |

TABLE 6
Nucleic Acid and Amino Acid Framework Sequences of Anti-RAN Antibodies

| Clone | region | FR1 | FR2 | FR3 | FR4 |
|---|---|---|---|---|---|
| 27B11.A7 | VH | EVQLQESGGGS | MSWVRQTPD | FYPDSVKG | WGQGTSVIVS |
| | | VQPGGSLKLSC | KRLELVTT | RFTISRDNA | S (SEQ ID NO: |
| | | AAS (SEQ ID | (SEQ ID NO: | KNALYLQM | 179) |
| | | NO: 155) | 163) | SSLKSDDTA | |
| | | | | MYYC (SEQ | |
| | | | | ID NO: 171) | |
| 27B11.A7 | VL | DIVMSQFPSSL | LAWYQQKPG | TRESGVPDR | FGGGTKLEIK |
| | | AVSAGDKVTM | QSPKLLIY | FTGSRSGTD | (SEQ ID NO: |
| | | SCKSS (SEQ ID | (SEQ ID NO: | FTLTISSVQ | 180) |
| | | NO: 156) | 164) | AEDLAVYY | |
| | | | | C (SEQ ID | |
| | | | | NO: 172) | |
| 23H2.D1.B5 | VH | EVQLQESGGGS | MSWVRQTPD | YYPDSVKG | WGQGTSVTVS |
| | | VQPGGALQLSC | KRLEMVAT | RFIISRDNA | S (SEQ ID NO: |
| | | AAS (SEQ ID | (SEQ ID NO: | KNTLYLQM | 181) |
| | | NO: 157) | 165) | SSLKSEDTA | |
| | | | | MYYC (SEQ | |
| | | | | ID NO: 173) | |
| 23H2.D1.B5 | VL | DIVMSQSPSSL | LAWYQQKPG | TRESGVPDR | FGGGTKLEIK |
| | | AVSEGEKVTLT | QPPKLLIY | FTGSGYGT | (SEQ ID NO: |
| | | CKSS (SEQ ID | (SEQ ID NO: | DFTLTISSV | 180) |
| | | NO: 158) | 166) | QAEDLAVY | |
| | | | | YC (SEQ ID | |
| | | | | NO: 174) | |
| 16A3.C8 | VH | EVQLQESGSEV | LHWVKQRPG | GYDEKFRT | WGQGTSVTVS |
| | | VRPGASVKLSC | QGLEWIGN | KATVTVDT | T (SEQ ID NO: |
| | | KAS (SEQ ID | (SEQ ID NO: | SSSTAYMQ | 183) |
| | | NO: 159) | 167) | LSSLTTEDS | |
| | | | | AVYYC | |
| | | | | (SEQ ID NO: | |
| | | | | 175) | |
| 16A3.C8 | VL | QIVLTQSPEILS | MHWYQQKSG | KLASGVPA | FGSGTKLEIK |
| | | ASPGEKVTMT | TSPKRWIY | RFSGSGSGT | (SEQ ID NO: |
| | | CNAT (SEQ ID | (SEQ ID NO: | SYSLTISSM | 184) |
| | | NO: 160) | 168) | EAEDAAAY | |
| | | | | YC (SEQ ID | |
| | | | | NO: 176) | |
| HL2362- | VH | GAGGTTCAGC | TGGGTGAAAC | AAGGCCAC | TGGGGTCAAG |
| 2G4 | | TGCAGCAGTC | AGAGGCCTG | TATGACTG | GAACCTCTGT |
| | | TGGGGCAGAA | AACAGGGCCT | CAGACACA | CACCGTCTCC |
| | | CTTGTGAGGT | GGAGTGGATT | TCCTCCAA | TCA (SEQ ID |
| | | CAGGGGCCTC | GGA (SEQ ID | CACAGCCT | NO: 193) |
| | | AGTCAAGTTG | NO: 189) | ACCTGCAG | |
| | | TCCTGCACAG | | CTCAGCAG | |
| | | CTTCTGGCTTC | | CCTGACAT | |
| | | AACATTAGA | | CTGAGGAC | |
| | | (SEQ ID NO: | | ACTGCCGT | |
| | | 187) | | CTATTACT | |
| | | | | GTAATGCA | |
| | | | | (SEQ ID NO: | |
| | | | | 191) | |
| | | EVQLQQSGAEL | WVKQRPEQG | KATMTADT | WGQGTSVTVS |
| | | VRSGASVKLSC | LEWIG (SEQ | SSNTAYLQL | S (SEQ ID NO: |
| | | TASGFNIR | ID NO: 169) | SSLTSEDTA | 181) |
| | | (SEQ ID NO: | | VYYCNA | |
| | | 161) | | (SEQ ID NO: | |
| | | | | 177) | |
| HL2362- | VL | GATGTTTTGAT | TGGTACCTGC | GGGGTCCC | TTCGGTGGAG |
| 2G4 | | GACCCAAACT | AGAAACCAG | AGACAGGT | GCACCAAGCT |
| | | CCACTCTCCCT | GCCAGTCTCC | TCAGTGGC | GGAAATCAA |
| | | GCCTGTCAGT | AAAGCTCCTG | AGTGGATC | A (SEQ ID NO: |
| | | CTTGGAGATC | ATCTAC (SEQ | AGGGACAG | 194) |
| | | AAGCCTCCAT | ID NO: 190) | ATTTCACA | |
| | | CTCTTGC (SEQ | | CTCAAGAT | |
| | | ID NO: 188) | | CAGCAGAG | |
| | | | | TGGAGGCT | |
| | | | | GATGATCT | |
| | | | | GGGAGTTT | |
| | | | | ATTACTGC | |
| | | | | (SEQ ID NO: | |
| | | | | 192) | |
| | | DVLMTQTPLSL | WYLQKPGQSP | GVPDRFSGS | FGGGTKLEIK |
| | | PVSLGDQASIS | KLLIY (SEQ ID | GSGTDFTLK | (SEQ ID NO: |
| | | C (SEQ ID NO: | NO: 170) | ISRVEADDL | 180) |
| | | 162) | | GVYYC | |
| | | | | (SEQ ID NO: | |
| | | | | 178) | |

TABLE 7
Constant Region Sequences

| Clone | region | Amino acid sequence | Nucleic acid sequence |
|---|---|---|---|
| HL2362- | Heavy chain | AKTTAPSVYPLAPVCGDTT | GCCAAAACAACAGCCCC |
| 2G4 | constant region | GSSVTLGCLVKGYFPEPVT | ATCGGTCTATCCACTGGC |
| | | LTWNSGSLSSGVHTFPAVL | CCCTGTGTGTGGAGATAC |
| | | QSDLYTLSSSVTVTSSTWP | AACTGGCTCCTCGGTGAC |
| | | SQSITCNVAHPASSTKVDK | TCTAGGATGCCTGGTCAA |
| | | KIEPRGPTIKPCPPCKCPAP | GGGTTATTTCCCTGAGCC |
| | | NLLGGPSVFIFPPKIKDVLM | AGTGACCTTGACCTGGA |
| | | ISLSPIVTCVVVDVSEDDPD | ACTCTGGATCCCTGTCCA |
| | | VQISWFVNNVEVHTAQTQ | GTGGTGTGCACACCTTCC |
| | | THREDYNSTLRVVSALPIQ | CAGCTGTCCTGCAGTCTG |
| | | HQDWMSGKEFKCKVNNK | ACCTCTACACCCTCAGCA |
| | | DLPAPIERTISKPKGSVRAP | GCTCAGTGACTGTAACCT |
| | | QVYVLPPPEEEMTKKQVT | CGAGCACCTGGCCCAGC |
| | | LTCMVTDFMPEDIYVEWT | CAGTCCATCACCTGCAAT |
| | | NNGKTELNYKNTEPVLDS | GTGGCCCACCCGGCAAG |
| | | DGSYFMYSKLRVEKKNW | CAGCACCAAGGTGGACA |
| | | VERNSYSCSVVHEGLHNH | AGAAAATTGAGCCCAGA |
| | | HTTKSFSRTPGK (SEQ ID | GGGCCCACAATCAAGCC |
| | | NO: 195) | CTGTCCTCCATGCAAATG |
| | | | CCCAGCACCTAACCTCTT |
| | | | GGGTGGACCATCCGTCTT |
| | | | CATCTTCCCTCCAAAGAT |
| | | | CAAGGATGTACTCATGA |
| | | | TCTCCCTGAGCCCCATAG |
| | | | TCACATGTGTGGTGGTGG |
| | | | ATGTGAGCGAGGATGAC |
| | | | CCAGATGTCCAGATCAG |
| | | | CTGGTTTGTGAACAACGT |
| | | | GGAAGTACACACAGCTC |
| | | | AGACACAAACCCATAGA |
| | | | GAGGATTACAACAGTAC |
| | | | TCTCCGGGTGGTCAGTGC |
| | | | CCTCCCCATCCAGCACCA |
| | | | GGACTGGATGAGTGGCA |
| | | | AGGAGTTCAAATGCAAG |
| | | | GTCAACAACAAAGACCT |
| | | | CCCAGCGCCCATCGAGA |
| | | | GAACCATCTCAAAACCC |
| | | | AAAGGGTCAGTAAGAGC |
| | | | TCCACAGGTATATGTCTT |
| | | | GCCTCCACCAGAAGAAG |
| | | | AGATGACTAAGAAACAG |
| | | | GTCACTCTGACCTGCATG |
| | | | GTCACAGACTTCATGCCT |
| | | | GAAGACATTTACGTGGA |
| | | | GTGGACCAACAACGGGA |
| | | | AAACAGAGCTAAACTAC |
| | | | AAGAACACTGAACCAGT |
| | | | CCTGGACTCTGATGGTTC |
| | | | TTACTTCATGTACAGCAA |
| | | | GCTGAGAGTGGAAAAGA |
| | | | AGAACTGGGTGGAAAGA |
| | | | AATAGCTACTCCTGTTCA |
| | | | GTGGTCCACGAGGGTCT |
| | | | GCACAATCACCACACGA |
| | | | CTAAGAGCTTCTCCCGGA |
| | | | CTCCGGGTAAA (SEQ ID |
| | | | NO: 197) |
| HL2362- | Light chain | RADAAPTVSIFPPSSEQLTS | CGGGCTGATGCTGCACC |
| 2G4 | constant region | GGASVVCFLNNFYPKDINV | AACTGTATCCATCTTCCC |
| | | KWKIDGSERQNGVLNSWT | ACCATCCAGTGAGCAGT |
| | | DQDSKDSTYSMSSTLTLTK | TAACATCTGGAGGTGCCT |
| | | DEYERHNSYTCEATHKTST | CAGTCGTGTGCTTCTTGA |
| | | SPIVKSFNRNEC (SEQ ID | ACAACTTCTACCCCAAA |
| | | NO: 196) | GACATCAATGTCAAGTG |
| | | | GAAGATTGATGGCAGTG |
| | | | AACGACAAAATGGCGTC |
| | | | CTGAACAGTTGGACTGA |
| | | | TCAGGACAGCAAAGACA |
| | | | GCACCTACAGCATGAGC |
| | | | AGCACCCTCACGTTGACC |
| | | | AAGGACGAGTATGAACG |
| | | | ACATAACAGCTATACCT |
| | | | GTGAGGCCACTCACAAG |
| | | | ACATCAACTTCACCCATT |
| | | | GTCAAGAGCTTCAACAG |
| | | | GAATGAGTGT (SEQ ID |
| | | | NO: 198) |

In some embodiments, antibody clone 27B11.A7 binds to polyGA. In some embodiments, clone 27B11.A7 is an IgG1 antibody. In some embodiments, antibody clone 23H2.D1.B5 binds to polyGA. In some embodiments, antibody clone 23H2.D1.B5 is an IgG3 antibody. In some embodiments, antibody clone 16A3.C8 binds to polySer. In some embodiments, antibody clone 16A3.C8 is an IgG1 antibody. In some embodiments, antibody clone HL2362-2G4 binds to polyPR. In some embodiments, antibody clone HL2362-2G4 is IgG2A kappa antibody.

Anti-RAN antibodies may be used to treat, or assist in the treatment of, one or more symptoms of a disease associated with RAN proteins. In some embodiments, the disease associated with RAN proteins is selected from the group consisting of: amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE). In a specific embodiment, the neurological disease associated with RAN proteins is Alzheimer's Disease (AD).

In some embodiments, the anti-RAN antibodies may be used to treat, or assist in the treatment of, one or more symptoms of a disease associated with RAN proteins, for example by administering a therapeutically effective amount of one or more anti-RAN antibodies to a subject diagnosed as having one or more symptoms of a disease associated with RAN proteins (e.g., the early stages of Alzheimer's disease) or being at risk of developing a disease associated with RAN proteins (e.g., based on one or more assays described in this application). In some embodiments, one or more of the anti-RAN antibody or antigen binding fragments disclosed herein are administered to a subject, wherein the subject has been characterized as having a disease associated with RAN proteins by the detection of at least one RAN protein in a biological sample obtained from the subject.

### Anti-RAN Antibody Production

Typically, polyclonal antibodies are produced by inoculation of a suitable mammal, such as a mouse, rabbit or goat. An antigen is injected into the mammal. This induces the B-lymphocytes to produce IgG immunoglobulins specific for the antigen. This polyclonal IgG is purified from the mammal's serum. Monoclonal antibodies are generally produced by a single cell line (e.g., a hybridoma cell line). In some embodiments, an anti-RAN antibody is purified (e.g., isolated from serum).

Exemplary anti-RAN antibodies disclosed herein were produced using the antigens set forth in Table 8. In some embodiments, an antigen comprises a RAN protein repeat sequence selected from poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 260); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 261); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), poly(PGGRGE) (SEQ ID NO: 258), poly(GRQRGVNT) (SEQ ID NO: 266), and poly(GSKHREAE) (SEQ ID NO: 267).

TABLE 8
Antigens for producing RAN antibodies

| Antibody | Antigen |
|---|---|
| 27B11.A7 | poly(GA)₃₀ (SEQ ID NO: 199) |
| 23H2.D1.B5 | poly(GA)₃₀ (SEQ ID NO: 199) |
| 16A3.C8 | H2N-SSSSSSSSSS(dPEG4)CKK-amide |
| | (SEQ ID NO: 201) |
| HL2362-2G4 | AC-RPRPRPRPRPRPRPRPC-amide (SEQ |
| | ID NO: 202) |

Numerous methods may be used for obtaining anti-RAN antibodies. For example, antibodies can be produced using recombinant DNA methods. Monoclonal antibodies may also be produced by generation of hybridomas (see, e.g., Kohler and Milstein (1975) Nature, 256: 495-499) in accordance with known methods. Hybridomas formed in this manner are then screened using standard methods, such as enzyme-linked immunosorbent assay (ELISA; e.g., RCA-based ELISA or rtPCR-based ELISA) and surface plasmon resonance (e.g., OCTET or BIACORE) analysis, to identify one or more hybridomas that produce an antibody that specifically binds with a specified antigen. Any form of the specified antigen (e.g., a RAN protein) may be used as the immunogen, e.g., recombinant antigen, naturally occurring forms, any variants or fragments thereof. One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228: 1315-1317; Clackson et al. (1991) Nature, 352: 624-628; Marks et al. (1991) J. Mol. Biol., 222: 581-597WO92/18619; WO 91/17271; WO 92/20791; WO 92/15679; WO 93/01288; WO 92/01047; WO 92/09690; and WO 90/02809.

In another embodiment, a monoclonal antibody is obtained from the non-human animal, and then modified, e.g., made chimeric, using recombinant DNA techniques known in the art. A variety of approaches for making chimeric antibodies have been described. See, e.g., Morrison et al., Proc. Natl. Acad. Sci. U.S.A. 81:6851, 1985; Takeda et al., Nature 314:452, 1985, Cabilly at al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

Antibodies can also be humanized by methods known in the art. For example, monoclonal antibodies with a desired binding specificity can be commercially humanized (Scotgene, Scotland; and Oxford Molecular, Palo Alto, Calif.). Fully humanized antibodies, such as those expressed in transgenic animals are within the scope of the invention (see, e.g., Green et al. (1994) Nature Genetics 7, 13; and U.S. Pat. Nos. 5,545,806 and 5,569,825). For additional antibody production techniques, see, Antibodies: A Laboratory Manual, Second Edition. Edited by Edward A. Greenfield, Dana-Farber Cancer Institute, ©2014. The present disclosure is not necessarily limited to any particular source, method of production, or other special characteristics of an antibody.

Some aspects of the present disclosure relate to isolated cells (e.g., host cells) transformed with a polynucleotide or vector. Host cells may be a prokaryotic or eukaryotic cell. The polynucleotide or vector which is present in the host cell may either be integrated into the genome of the host cell or it may be maintained extrachromosomally. The host cell can be any prokaryotic or eukaryotic cell, such as a bacterial, insect, fungal, plant, animal or human cell. In some embodiments, fungal cells are, for example, those of the genus Saccharomyces, in particular those of the species S. cerevisiae. The term “prokaryotic” includes all bacteria which can be transformed or transfected with a DNA or RNA molecules for the expression of an antibody or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” includes yeast, higher plants, insects and vertebrate cells, e.g., mammalian cells, such as NSO and CHO cells. Depending upon the host employed in a recombinant production procedure, the antibodies or immunoglobulin chains encoded by the polynucleotide may be glycosylated or may be non-glycosylated. Antibodies or the corresponding immunoglobulin chains may also include an initial methionine amino acid residue.

In some embodiments, once a vector has been incorporated into an appropriate host, the host may be maintained under conditions suitable for high level expression of the nucleotide sequences, and, as desired, the collection and purification of the immunoglobulin light chains, heavy chains, light/heavy chain dimers or intact antibodies, antigen binding fragments or other immunoglobulin forms may follow; see, Beychok, Cells of Immunoglobulin Synthesis, Academic Press, N.Y., (1979). Thus, polynucleotides or vectors are introduced into the cells which in turn produce the antibody or antigen binding fragments. Furthermore, transgenic animals, preferably mammals, comprising the aforementioned host cells may be used for the large scale production of the antibody or antibody fragments.

The transformed host cells can be grown in fermenters and cultured according to techniques known in the art to achieve optimal cell growth. Once expressed, the whole antibodies, their dimers, individual light and heavy chains, other immunoglobulin forms, or antigen binding fragments, can be purified according to standard procedures of the art, including ammonium sulfate precipitation, affinity columns, column chromatography, gel electrophoresis and the like; see, Scopes, “Protein Purification”, Springer Verlag, N.Y. (1982). The antibody or antigen binding fragments can then be isolated from the growth medium, cellular lysates, or cellular membrane fractions. The isolation and purification of the, e.g., microbially expressed antibodies or antigen binding fragments may be by any conventional means such as, for example, preparative chromatographic separations and immunological separations such as those involving the use of monoclonal or polyclonal antibodies directed, e.g., against the constant region of the antibody.

Aspects of the disclosure relate to a hybridoma, which provides an indefinitely prolonged source of monoclonal antibodies. As used herein, “hybridoma cell” refers to an immortalized cell derived from the fusion of B lymphoblasts with a myeloma fusion partner. For preparing monoclonal antibody-producing cells (e.g., hybridoma cells), an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Kochler and Milstein (Nature 256:495 (1975)). As a fusion promoter, for example, polyethylene glycol (PEG) or Sendai virus (HVJ) is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

As an alternative to obtaining immunoglobulins directly from the culture of hybridomas, immortalized hybridoma cells can be used as a source of rearranged heavy chain and light chain loci for subsequent expression and/or genetic manipulation. Rearranged antibody genes can be reverse transcribed from appropriate mRNAs to produce cDNA. If desired, the heavy chain constant region can be exchanged for that of a different isotype or eliminated altogether. The variable regions can be linked to encode single chain Fv regions. Multiple Fv regions can be linked to confer binding ability to more than one target or chimeric heavy and light chain combinations can be employed. Any appropriate method may be used for cloning of antibody variable regions and generation of recombinant antibodies.

In some embodiments, an appropriate nucleic acid that encodes variable regions of a heavy and/or light chain is obtained and inserted into an expression vectors which can be transfected into standard recombinant host cells. A variety of such host cells may be used. In some embodiments, mammalian host cells may be advantageous for efficient processing and production. Typical mammalian cell lines useful for this purpose include CHO cells, 293 cells, or NSO cells. The production of the antibody or antigen binding fragment may be undertaken by culturing a modified recombinant host under culture conditions appropriate for the growth of the host cells and the expression of the coding sequences. The antibodies or antigen binding fragments may be recovered by isolating them from the culture. The expression systems may be designed to include signal peptides so that the resulting antibodies are secreted into the medium; however, intracellular production is also possible.

The disclosure also includes a polynucleotide encoding at least a variable region of an immunoglobulin chain of the antibodies described herein. In some embodiments, the variable region encoded by the polynucleotide comprises at least one complementarity determining region (CDR) of the VH and/or VL of the variable region of the antibody produced by any one of the above described hybridomas.

Polynucleotides encoding antibody or antigen binding fragments may be, e.g., DNA, cDNA, RNA or synthetically produced DNA or RNA or a recombinantly produced chimeric nucleic acid molecule comprising any of those polynucleotides either alone or in combination. In some embodiments, a polynucleotide is part of a vector. Such vectors may comprise further genes such as marker genes which allow for the selection of the vector in a suitable host cell and under suitable conditions.

In some embodiments, a polynucleotide is operatively linked to expression control sequences allowing expression in prokaryotic or eukaryotic cells. Expression of the polynucleotide comprises transcription of the polynucleotide into a translatable mRNA. Regulatory elements ensuring expression in eukaryotic cells, preferably mammalian cells, are well known to those skilled in the art. They may include regulatory sequences that facilitate initiation of transcription and optionally poly-A signals that facilitate termination of transcription and stabilization of the transcript. Additional regulatory elements may include transcriptional as well as translational enhancers, and/or naturally associated or heterologous promoter regions. Possible regulatory elements permitting expression in prokaryotic host cells include, e.g., the PL, Lac, Trp or Tac promoter in E. coli, and examples of regulatory elements permitting expression in eukaryotic host cells are the AOX1 or GAL1 promoter in yeast or the CMV-promoter, SV40-promoter, RSV-promoter (Rous sarcoma virus), CMV-enhancer, SV40-enhancer or a globin intron in mammalian and other animal cells.

Beside elements which are responsible for the initiation of transcription such regulatory elements may also include transcription termination signals, such as the SV40-poly-A site or the tk-poly-A site, downstream of the polynucleotide. Furthermore, depending on the expression system employed, leader sequences capable of directing the polypeptide to a cellular compartment or secreting it into the medium may be added to the coding sequence of the polynucleotide and are well known in the art. The leader sequence(s) is (are) assembled in appropriate phase with translation, initiation and termination sequences, and preferably, a leader sequence capable of directing secretion of translated protein, or a portion thereof, into, for example, the extracellular medium. Optionally, a heterologous polynucleotide sequence can be used that encode a fusion protein including a C- or N-terminal identification peptide imparting desired characteristics, e.g., stabilization or simplified purification of expressed recombinant product.

In some embodiments, polynucleotides encoding at least the variable domain of the light and/or heavy chain may encode the variable domains of both immunoglobulin chains or only one. Likewise, polynucleotides may be under the control of the same promoter or may be separately controlled for expression. Furthermore, some aspects relate to vectors, particularly plasmids, cosmids, viruses and bacteriophages used conventionally in genetic engineering that comprise a polynucleotide encoding a variable domain of an immunoglobulin chain of an antibody or antigen binding fragment; optionally in combination with a polynucleotide that encodes the variable domain of the other immunoglobulin chain of the antibody.

In some embodiments, expression control sequences are provided as eukaryotic promoter systems in vectors capable of transforming or transfecting eukaryotic host cells, but control sequences for prokaryotic hosts may also be used. Expression vectors derived from viruses such as retroviruses, vaccinia virus, adeno-associated virus, herpes viruses, or bovine papilloma virus, may be used for delivery of the polynucleotides or vector into targeted cell population (e.g., to engineer a cell to express an antibody or antigen binding fragment). A variety of appropriate methods can be used to construct recombinant viral vectors. In some embodiments, polynucleotides and vectors can be reconstituted into liposomes for delivery to target cells. The vectors containing the polynucleotides (e.g., the heavy and/or light variable domain(s) of the immunoglobulin chains encoding sequences and expression control sequences) can be transferred into the host cell by suitable methods, which vary depending on the type of cellular host.

### Modifications

Some aspects of the disclosure relate to antibody-drug conjugates targeted against one or more RAN proteins. As used herein, “antibody drug conjugate” refers to molecules comprising an antibody, or antigen binding fragment thereof, linked to a targeted molecule (e.g., a biologically active molecule, such as a therapeutic molecule, and/or a detectable label). Accordingly, in some embodiments, antibodies or antigen binding fragments of the disclosure may be modified with a detectable label, including, but not limited to, an enzyme, prosthetic group, fluorescent material, luminescent material, bioluminescent material, radioactive material, positron emitting metal, nonradioactive paramagnetic metal ion, and affinity label for detection and isolation of one or more RAN proteins. The detectable substance may be coupled or conjugated either directly to the polypeptides of the disclosure or indirectly, through an intermediate (such as, for example, a linker known in the art) using techniques known in the art. Non-limiting examples of suitable enzymes include horseradish peroxidase, alkaline phosphatase, β-galactosidase, glucose oxidase, or acetylcholinesterase; non-limiting examples of suitable prosthetic group complexes include streptavidin/biotin and avidin/biotin; non-limiting examples of suitable fluorescent materials include biotin, umbelliferone, fluorescein, fluorescein isothiocyanate, rhodamine, dichlorotriazinylamine fluorescein, dansyl chloride, or phycoerythrin; an example of a luminescent material includes luminol; non-limiting examples of bioluminescent materials include luciferase, luciferin, and aequorin; and examples of suitable radioactive material include a radioactive metal ion, e.g., alpha-emitters or other radioisotopes such as, for example, iodine (¹³¹I, ¹²⁵I, ¹²³I, ¹²¹I) carbon (¹⁴C), sulfur (³⁵S), tritium (³H), indium (¹¹⁵mIn, ¹¹³mIn, ¹¹²In, ¹¹¹In), and technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe), flourine (¹⁸F), ¹⁵³Sm, Lu, ¹⁵⁹Gd, ¹⁴⁹Pm, ¹⁴⁰La, ¹⁷⁵Yb, ¹⁶⁶Ho, ⁹⁰Y, ⁴⁷Sc, ⁸⁶R, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, and tin (¹¹³Sn, ¹¹⁷Sn). The detectable substance may be coupled or conjugated either directly to the anti-RAN antibodies or antigen-binding fragments of the disclosure or indirectly, through an intermediate (such as, for example, a linker known in the art) using techniques known in the art. Anti-RAN antibodies conjugated to a detectable substance may be used for diagnostic assays as described herein.

In some embodiments, antibodies or antigen binding fragments of the disclosure may be modified with a therapeutic moiety (e.g., therapeutic agent). In some embodiments, the antibody is coupled to the targeted agent via a linker. As used herein, the term “linker” refers to a molecule or sequence, such as an amino acid sequence, that attaches, as in a bridge, one molecule or sequence to another molecule or sequence. “Linked,” “conjugated,” or “coupled” means attached or bound by covalent bonds, or non-covalent bonds, or other bonds, such as van der Waals forces. Antibodies described by the disclosure can be linked to the targeted agent (e.g., therapeutic moiety or detectable moiety) directly, e.g., as a fusion protein with protein or peptide detectable moieties (with or without an optional linking sequence, e.g., a flexible linker sequence) or via a chemical coupling moiety. A number of such coupling moieties are known in the art, e.g., a peptide linker or a chemical linker, e.g., as described in International Patent Application Publication No. WO 2009/036092. In some embodiments, the linker is a flexible amino acid sequence. Examples of flexible amino acid sequences include glycine and serine rich linkers, which comprise a stretch of two or more glycine residues. In some embodiments, the linker is a photolinker. Examples of photolinkers include ketyl-reactive benzophenone (BP), anthraquinone (AQ), nitrene-reactive nitrophenyl azide (NPA), and carbene-reactive phenyl-(trifluoromethyl)diazirine (PTD).

### Pharmaceutical Compositions

In some aspects, the disclosure relates to pharmaceutical compositions comprising anti-RAN antibodies or antigen binding fragments. In some embodiments, the composition comprises an anti-RAN antibody and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical compositions can be prepared as described below. The active ingredients may be admixed or compounded with any conventional, pharmaceutically acceptable carrier or excipient. The compositions may be sterile.

Typically, pharmaceutical compositions are formulated for delivering an effective amount of an agent (e.g., an anti-RAN antibody). In general, an “effective amount” of an active agent refers to an amount sufficient to elicit the desired biological response (e.g., ameliorating one or more symptoms of Alzheimer's disease). An effective amount of an agent may vary depending on such factors as the desired biological endpoint, the pharmacokinetics of the compound, the disease being treated (e.g., Alzheimer's disease, repeat expansion diseases), the mode of administration, and the patient.

A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. See, for example, REMINGTON'S PHARMACEUTICAL SCIENCES, 18th Ed. (1990).

It will be understood by those skilled in the art that any mode of administration, vehicle or carrier conventionally employed and which is inert with respect to the active agent may be utilized for preparing and administering the pharmaceutical compositions of the present disclosure. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the disclosure, will experience no difficulty in determining suitable and appropriate vehicles, excipients and carriers or in compounding the active ingredients therewith to form the pharmaceutical compositions of the disclosure.

An effective amount, also referred to as a therapeutically effective amount, of a compound (e.g., an anti-RAN antibody) is an amount sufficient to ameliorate at least one adverse effect associated with a disease associated with RAN proteins, such as, e.g., memory loss, cognitive impairment, loss of coordination, speech impairment, etc. In some embodiments, the neurological disease associated with RAN proteins is selected from the group consisting of: amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE). In a specific embodiment, the neurological disease associated with RAN proteins is Alzheimer's Disease (AD). The therapeutically effective amount to be included in pharmaceutical compositions depends, in each case, upon several factors, e.g., the type, size and condition of the patient to be treated, the intended mode of administration, the capacity of the patient to incorporate the intended dosage form, etc. Generally, an amount of active agent is included in each dosage form to provide from about 0.1 to about 250 mg/kg, and preferably from about 0.1 to about 100 mg/kg. One of ordinary skill in the art would be able to determine empirically an appropriate therapeutically effective amount.

Combined with the teachings provided herein, by choosing among the various active compounds and weighing factors such as potency, relative bioavailability, patient body weight, severity of adverse side-effects and selected mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic agent being administered, the size of the subject, or the severity of the disease or condition. One of ordinary skill in the art can empirically determine the effective amount of a particular nucleic acid and/or other therapeutic agent without necessitating undue experimentation.

In some cases, compounds of the disclosure are prepared in a colloidal dispersion system. Colloidal dispersion systems include lipid-based systems including oil-in-water emulsions, micelles, mixed micelles, and liposomes. In some embodiments, a colloidal system of the disclosure is a liposome. Liposomes are artificial membrane vessels which are useful as a delivery vector in vivo or in vitro. It has been shown that large unilamellar vesicles (LUVs), which range in size from 0.2-4.0 μm can encapsulate large macromolecules.

Liposomes may be targeted to a particular tissue by coupling the liposome to a specific ligand such as a monoclonal antibody, sugar, glycolipid, or protein. Ligands which may be useful for targeting a liposome to, for example, an smooth muscle cell include, but are not limited to: intact or fragments of molecules which interact with smooth muscle cell specific receptors and molecules, such as antibodies, which interact with the cell surface markers of cancer cells. Such ligands may easily be identified by binding assays well known to those of skill in the art. In still other embodiments, the liposome may be targeted to a tissue by coupling it to an antibody known in the art.

Compounds described by the disclosure may be administered alone (e.g., in saline or buffer) or using any delivery vehicle known in the art. For instance the following delivery vehicles have been described: cochleates; Emulsomes; ISCOMs; liposomes; live bacterial vectors (e.g., Salmonella, Escherichia coli, Bacillus Calmette-Guerin, Shigella, Lactobacillus); live viral vectors (e.g., Vaccinia, adenovirus, Herpes simplex); microspheres; nucleic acid vaccines; polymers (e.g., carboxymethylcellulose, chitosan); polymer rings; proteosomes; sodium fluoride; transgenic plants; virosomes; and, virus-like particles.

The formulations of the disclosure are administered in pharmaceutically acceptable solutions, which may routinely contain pharmaceutically acceptable concentrations of salt, buffering agents, preservatives, compatible carriers, adjuvants, and optionally other therapeutic ingredients.

The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present disclosure, and with each other, in a manner such that there is no interaction which would substantially impair the desired pharmaceutical efficiency.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In addition to the formulations described herein, the compounds may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see, Langer R (1990) Science 249:1527-1533, which is incorporated herein by reference.

The compounds may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories.

### Administration

A therapeutic agent may be delivered by any suitable modality known in the art. In some embodiments, a therapeutic agent (e.g., a protein, antibody, interfering nucleic acid, etc.) is delivered to a subject by a vector, such as a viral vector (e.g., adenovirus vector, recombinant adeno-associated virus vector (rAAV vector), lentiviral vector, etc.) or a plasmid-based vector. In some embodiments, a therapeutic agent is delivered to a subject (e.g., a subject having Alzheimer's disease characterized by expression of one or more RAN proteins) in a recombinant adeno-associated virus (rAAV) particle.

In some embodiments, a recombinant rAAV particle comprises a nucleic acid vector, such as a single-stranded (ss) or self-complementary (sc) AAV nucleic acid vector. In some embodiments, the nucleic acid vector comprises a transgene encoding an therapeutic agent as described herein (e.g., a protein, antibody, interfering nucleic acid, etc.), and one or more regions comprising inverted terminal repeat (ITR) sequences (e.g., wild-type ITR sequences or engineered ITR sequences) flanking the expression construct. In some embodiments, the nucleic acid is encapsidated by a viral capsid. In some embodiments, the transgene is operably linked to a promoter, for example a constitutive promoter or an inducible promoter. In some embodiments, the promoter is a tissue-specific (e.g., CNS-specific) promoter. In some embodiments, a rAAV particle comprises a viral capsid that has a tropism for CNS tissue, for example AAV9 capsid protein or AAV.PHPB capsid protein.

Aspects of the disclosure relate to the delivery of a therapeutically effective amount of a therapeutic agent to a subject. In some embodiments, a therapeutically effective amount is an amount effective in reducing repeat expansions in the subject. In some embodiments, a therapeutically effective amount is an amount effective in reducing the transcription of RNAs that produce RAN proteins in a subject. In certain embodiments, a therapeutically effective amount is an amount effective in reducing the translation of RAN proteins in a subject. In some embodiments, a therapeutically effective amount is an amount effective for treating Alzheimer's disease associated with repeat expansions. “Reducing” expression of a repeat sequence or RAN protein translation refers to a decrease in the amount or level of repeat sequence expression or RAN protein translation in a subject after administration of a therapeutic agent (and relative to the amount or level in the subject prior to the administration).

In certain embodiments, the effective amount is an amount effective in reducing the level of RAN proteins by at least 10%, at least 20%, at least 30%, at least 40%, at least 50%, at least 60%, at least 70%, at least 80%, at least 90%, at least 95%, or at least 98% (e.g., the level of RAN proteins relative to the level of RAN proteins in a cell or subject that has not been administered a therapeutic agent). In certain embodiments, the effective amount is an amount effective in reducing the translation of RAN proteins by at least 10%, at least 20%, at least 30%, at least 40%, at least 50%, at least 60%, at least 70%, at least 80%, at least 90%, at least 95%, or at least 98% (e.g., the level of RAN proteins relative the level of RAN proteins in a cell or subject that has not been administered a therapeutic agent).

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof. The exemplary liquid dosage forms in certain embodiments are formulated for ease of swallowing, or for administration via feeding tube.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Therapeutic agents described herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

A therapeutic agent can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a therapeutic agent required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of a compound described herein. In certain embodiments, when multiple doses are administered to a subject or applied to a biological sample, tissue, or cell, the frequency of administering the multiple doses to the subject or applying the multiple doses to the biological sample, tissue, or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample, tissue, or cell is one dose per day. In certain embodiments, the frequency of administering the multiple doses to the subject or applying the multiple doses to the biological sample, tissue, or cell is two doses per day. In certain embodiments, the frequency of administering the multiple doses to the subject or applying the multiple doses to the biological sample, tissue, or cell is three doses per day. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, eight months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Routes of administration include but are not limited to oral, parenteral, intravenous, intramuscular, intraperitoneal, intranasal, sublingual, intratracheal, inhalation, subcutaneous, ocular, vaginal, and rectal. Systemic routes include oral and parenteral. Several types of devices are regularly used for administration by inhalation. These types of devices include metered dose inhalers (MDI), breath-actuated MDI, dry powder inhaler (DPI), spacer/holding chambers in combination with MDI, and nebulizers.

In some embodiments, a treatment for a disease associated with RAN protein expression is administered to the central nervous system (CNS) of a subject in need thereof. As used herein, the “central nervous system (CNS)” refers to all cells and tissues of the brain and spinal cord of a subject, including but not limited to neuronal cells, glial cells, astrocytes, cerebrospinal fluid, etc. Modalities of administering a therapeutic agent to the CNS of a subject include direct injection into the brain (e.g., intracerebral injection, intraventricular injection, intraparenchymal injection, etc.), direct injection into the spinal cord of a subject (e.g., intrathecal injection, lumbar injection, etc.), or any combination thereof.

In some embodiments, a treatment as described by the disclosure is systemically administered to a subject, for example by intravenous injection. Systemically administered therapeutic molecules can be modified, in some embodiments, in order to improve delivery of the molecules to the CNS of a subject. Examples of modifications that improve CNS delivery of therapeutic molecules include but are not limited to co-administration or conjugation to blood brain barrier-targeting agents (e.g., transferrin, melanotransferrin, low-density lipoprotein (LDL), angiopeps, RVG peptide, etc., as disclosed by Georgieva et al. Pharmaceuticals 6(4): 557-583 (2014)), coadministration with BBB disrupting agents (e.g., bradykinins), and physical disruption of the BBB prior to administration (e.g., by MRI-Guided Focused Ultrasound), etc.

The following Examples are intended to illustrate the benefits of the present invention and to describe particular embodiments, but are not intended to exemplify the full scope of the invention. Accordingly, it will be understood that the Examples are not meant to limit the scope of the invention.

### EXAMPLES

Microsatellite repeat expansions cause more than forty inherited neurodegenerative and neuromuscular diseases. Repeat tract lengths are typically <30 in the general population, ˜30-40 in pre-mutation carriers, and can range from ˜40 to thousands of repeats in affected individuals, depending on the disease. Expansion mutations often undergo bidirectional transcription to generate sense and antisense transcripts that can form RNA foci. Repeat expansion RNAs can be translated by repeat-associated non-AUG translation (RAN) to produce polymeric proteins. These repeat expansions are generally difficult to detect by high-throughput sequencing.

### Example 1

Alzheimer's disease (AD) is a progressive dementia affecting approximately 10% of the general population 65 years of age or older. While mutations in specific genes including apolipoprotein (APP), and presenillin genes (PSEN1 and PSEN2) have been observed in a subset of AD cases, the causes of the vast majority of AD cases are currently unknown. AD is characterized by the accumulation of β-amyloid (Aβ) peptides and hyper-phosphorylated tau protein throughout patient autopsy brains. However, the weak correlation of Aβ deposition and cognitive decline, and the limited efficacy to date of approaches to target Aβ and tau indicate that other factors play an important role in AD.

This example describes identification of repeat expansions which contribute to Alzheimer's disease, dementia and other neurodegenerative diseases. These putative expansions may contribute to disease either from single unidentified repeat expansion mutations or from the combined effects of multiple genes, each with smaller pre-mutation lengths. Data indicates that repeat RNAs and/or RAN proteins produced from these expansion mutations contribute to disease by disrupting protein homeostasis, proteasome function and autophagy.

### Identification of RAN Protein Translation in AD Patient Samples

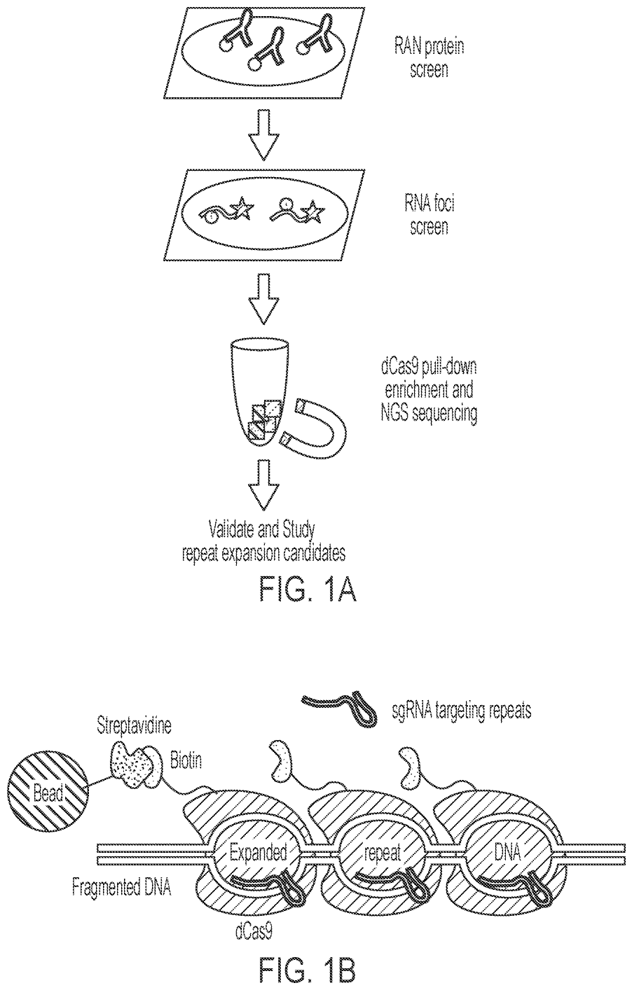

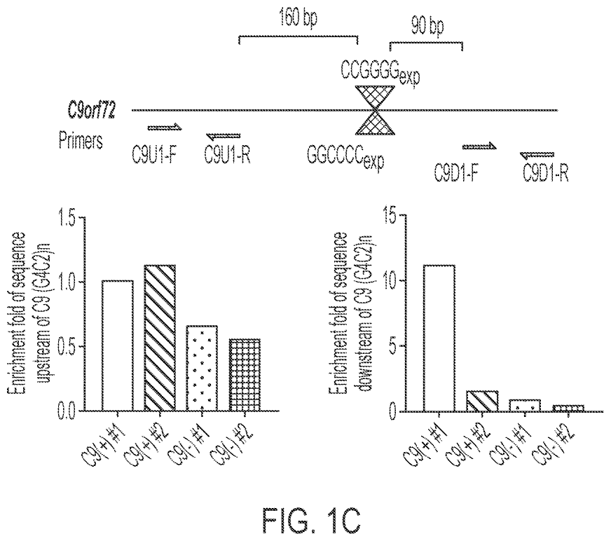

An antibody-based screening tool was developed in order to screen AD patient samples for RAN protein expression. FIGS. 1A and 1B show schematics of the screening protocol. First, a sample is contacted with a panel of antibodies that bind to a variety of repetitive peptide motifs to determine if RAN protein aggregates are present in the sample (FIG. 1A). This antibody-based approach identified positive RAN staining in 21/120 human AD autopsy brains that were examined. Briefly, both soluble and insoluble fractions of protein lysates extracted from frozen AD autopsy brain tissue were contacted with antibodies against various RAN proteins (e.g., α-polySer, α-polyGP, α-polyGA, α-polyGR, α-polyPR). Positive signal from these antibodies was found by an initial dot-blot screen of insoluble proteins from 21 of 120 AD cases compared to age-matched healthy controls.

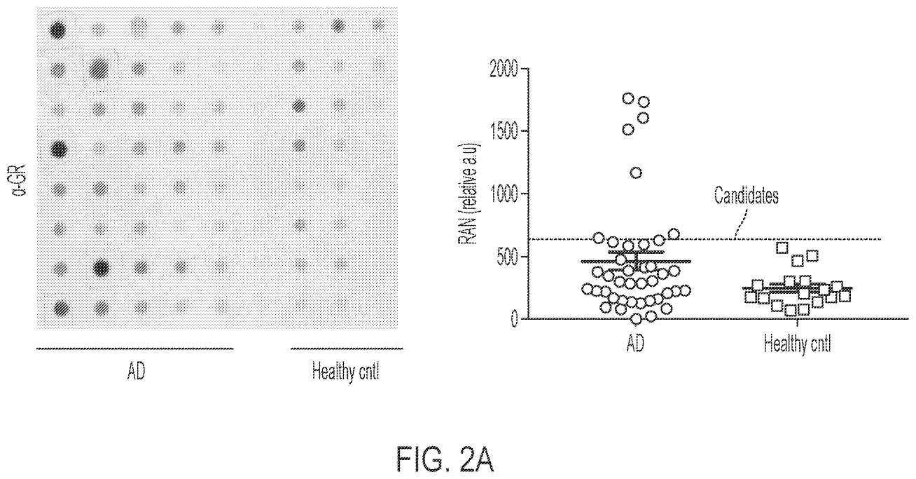

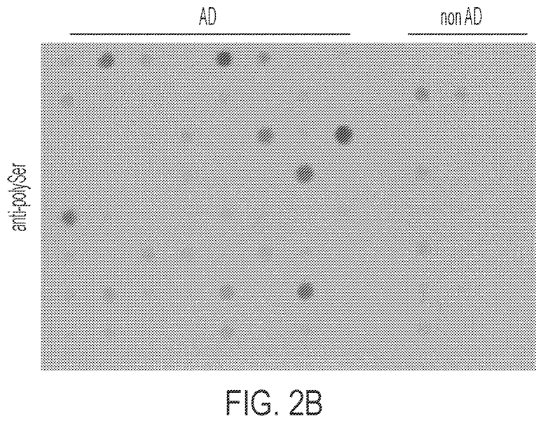

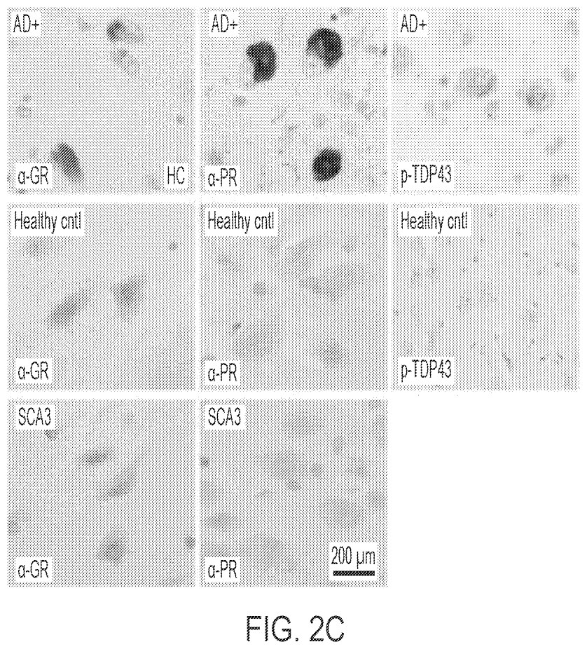

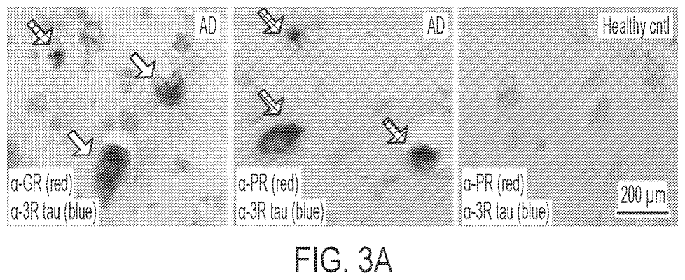

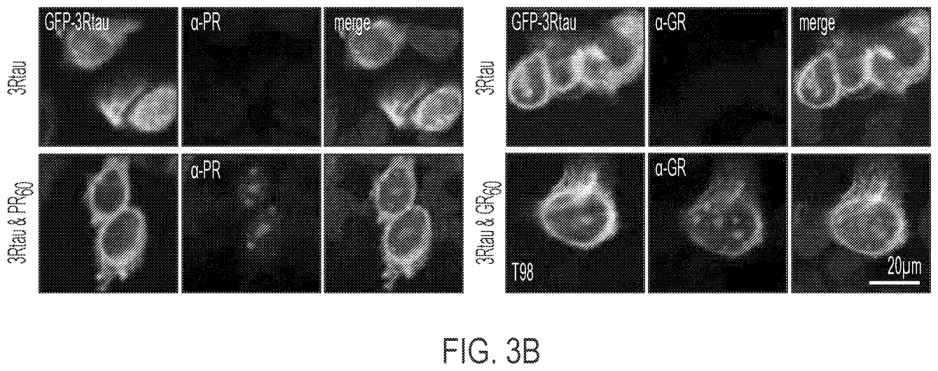

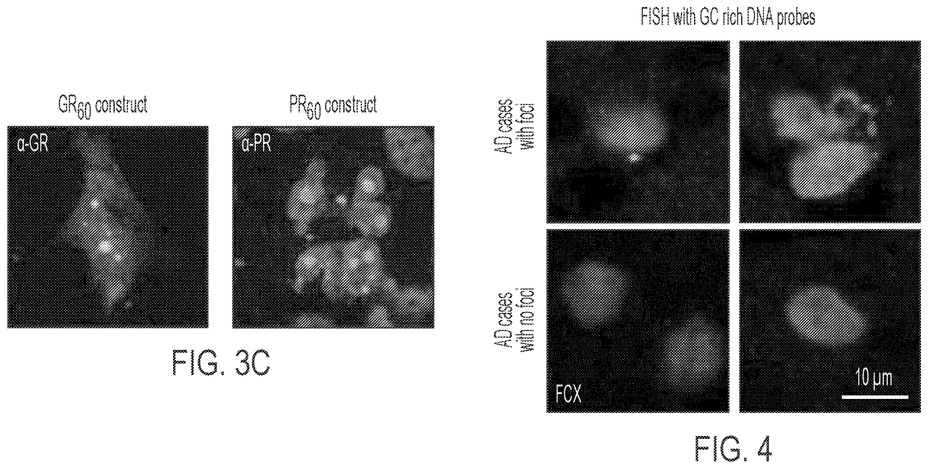

Dot blots and quantification of signals are shown for a subset of samples positive for α-polyGR and polySer staining (FIGS. 2A and 2B). Immunohistochemical (IHC) staining of hippocampal and frontal cortex sections from 20 candidate cases and ˜15 healthy and disease controls showed robust RAN positive signals in AD cases with no similar staining in age-matched healthy or disease controls. Example staining with α-polyGR and α-polyPR is shown in FIG. 2C. The α-polyGR and α-polyPR staining was not similar to staining for phosphorylated TDP43 (e.g., FIG. 2C). Additionally, double IHC staining for RAN-GR proteins and tau(3R) in AD hippocampus showed distinct patterns with overlapping and non-overlapping signals, denoted by arrows (FIG. 3A). Additional control IF experiments on cells transiently expressing 3Rtau and/or GR or PR RAN proteins show α-polyGR and α-polyPR recognize their corresponding tagged RAN proteins (FIG. 3C) but do not cross react with 3Rtau (FIG. 3B). Taken together, these data indicate that RAN staining is present in a subset of AD cases and these proteins accumulate in patterns that are distinct from pTDP43 and 3Rtau.

To further characterize these candidate RAN positive AD cases, the distribution of RAN staining was examined by IHC in various regions of AD autopsy brains. RAN protein distribution was compared with addition forms of tau protein (e.g., 4-repeat (4R) tau, phosphorylated tau) and Aβ. Additionally, RAN staining and distribution were compared with Braak scores to examine if and how RAN protein accumulation correlates with disease stage. Additionally, all RAN positive candidate cases were screened to eliminate cases with co-morbid known repeat expansions. All poly(GR) and poly(PR) RAN positive samples have been shown to be negative for the C9ORF72 expansion mutation.

Next, a RNA foci screening approach was used to identify the candidate RAN protein-encoding repeat expansion motifs, which were combined with biotin-tagged nuclease-deficient Cas9 (dCas9) to pull down the candidate repeat expansion mutations and corresponding flanking sequences from genomic DNA isolated from AD tissues samples positive for both RAN protein aggregates and RNA foci (FIG. 1B). Sequencing of the upstream and downstream regions flanking the repeat was used to identify the specific location of the repeat expansion.

The repeat motifs of the putative AD RAN proteins were used to identify all possible DNA sequences that could encode the RAN proteins. For example, all possible repeat motifs encoding GR, PR, and polySer are shown below in Tables 1, 2 and 3. Those skilled in the art will appreciate how to construct analogous tabulations of all possible nucleic acid sequences encoding the other RAN repeats identified herein including poly(CP), poly(GP), poly(G), poly(GA), poly(GD), poly(GE), poly(GQ), poly(GT), poly(L), poly(LP), poly(LPAC) (SEQ ID NO: 260), poly(LS), poly(P), poly(PA), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), or poly(PGGRGE) (SEQ ID NO: 258) using the standard vertebrate DNA translation code.

TABLE 1
All possible nucleic acid sequences encoding GR

| | | | |
|---|---|---|---|
| GGTCGT (SEQ ID | GGCCGT (SEQ ID | GGACGT (SEQ ID | GGGCGT (SEQ ID |
| NO: 82) | NO: 83) | NO: 84) | NO: 85) |
| GGTCGC (SEQ ID | GGCCGC (SEQ ID | GGACGC (SEQ ID | GGGCGC (SEQ ID |
| NO: 86) | NO: 87) | NO: 88) | NO: 89) |
| GGTCGA (SEQ ID | GGCCGA (SEQ ID | GGACGA (SEQ ID | GGGCGA (SEQ ID |
| NO: 90) | NO: 91) | NO: 92) | NO: 93) |
| GGTCGG (SEQ ID | GGCCGG (SEQ ID | GGACGG (SEQ ID | GGGCGG (SEQ ID |
| NO: 94) | NO: 95) | NO: 96) | NO: 97) |
| GGTAGA (SEQ ID | GGCAGA (SEQ ID | GGAAGA (SEQ ID | GGGAGA (SEQ ID |
| NO: 98) | NO: 99) | NO: 100) | NO: 101) |
| GGTAGG (SEQ ID | GGCAGG (SEQ ID | GGAAGG (SEQ ID | GGGAGG (SEQ ID |
| NO: 102) | NO: 103) | NO: 104) | NO: 105) |

TABLE 2
All possible nucleic acid sequences encoding PR

| | | | |
|---|---|---|---|
| CCTCGT (SEQ ID | CCCCGT (SEQ ID | CCACGT (SEQ ID | CCGCGT (SEQ ID |
| NO: 58) | NO: 59) | NO: 60) | NO: 61) |
| CCTCGC (SEQ ID | CCCCGC (SEQ ID | CCACGC (SEQ ID | CCGCGC (SEQ ID |
| NO: 62) | NO: 63) | NO: 64) | NO: 65) |
| CCTCGA (SEQ ID | CCCCGA (SEQ ID | CCACGA (SEQ ID | CCGCGA (SEQ ID |
| NO: 66) | NO: 67) | NO: 68) | NO: 69) |
| CCTCGG (SEQ ID | CCCCGG (SEQ ID | CCACGG (SEQ ID | CCGCGG (SEQ ID |
| NO: 70) | NO: 71) | NO: 72) | NO: 73) |
| CCTAGA (SEQ ID | CCCAGA (SEQ ID | CCAAGA (SEQ ID | CCGAGA (SEQ ID |
| NO: 74) | NO: 75) | NO: 76) | NO: 77) |
| CCTAGG (SEQ ID | CCCAGG (SEQ ID | CCAAGG (SEQ ID | CCGAGG (SEQ ID |
| NO: 78) | NO: 79) | NO: 80) | NO: 81) |

TABLE 3
All possible nucleic acid sequences
encoding polySer

| | | |
|---|---|---|
| TCT | TCC | TCA |
| TCG | AGT | AGC |

The sequences were used to design fluorophore-conjugated DNA probes that target all possible repeat motifs that could encode a given candidate RAN AD protein. Fluorescence in situ hybridization (FISH) screening of frozen AD brains using the RAN protein nucleic acid probes showed punctate RNA foci, a hallmark of repeat expansion diseases (FIG. 4). No similar RNA foci were found in controls or RAN-negative AD cases. Detection of RNA foci and RAN protein staining in candidate AD brain tissues demonstrates the presence of one or more novel AD repeat expansion mutations.

To identify the specific locus containing the repeat expansion mutations in RAN and RNA foci positive candidate AD cases, a pull-down assay is used to enrich the specific repeat expansion mutation and the corresponding flanking sequences using a biotin-tagged nuclease-deficient Cas9 (dCas9) approach (FIG. 1B). This dCas9-based enrichment tool pulls down and enriches specific DNA sequences by taking advantage of the rapid kinetics and high stability of single guide RNA/dCas9 (sgRNA-dCas9) complexes without the need to denature target DNA. Expanded repeats provide multiple binding sites for sgRNAs, thus increasing the probability of interaction between sgRNA-dCas9 complexes and expanded repeats compared to shorter repeat tracts (FIG. 1B).

Data indicates enrichment of the C9ORF72 G4C2 expansion mutation can be detected using this method. PCR data shows an enrichment for 5′ and 3′ sequences flanking the C9ORF72 G4C2 repeat in some of the C9(+) compared to C9(−) cases (FIG. 1C). For candidate AD cases, genomic repeat expansion containing DNA samples is enriched using a set of sgRNAs that target putative RAN expansion mutations that are predicted by IHC and RNA FISH experiments. Enriched and unenriched samples are then sequenced using next-generation sequencing techniques to identify repeat expansion loci that produce RAN proteins in AD.

### Example 2

### Molecular Pathways Affected by RAN Protein Translation

This example provides data indicating that RAN protein translation alters protein homeostasis in neurons and glia of subjects having AD and other CNS disorders. Repeat expansion patient iPSC-derived neurons and glia are used to study disruption of proteasome and autophagy pathways via proteasome activity, autophagic flux, proteasome and autophagy markers (e.g., p62, LC3, proteasome subunits) and transcriptomic analysis. To determine if repeat expansions accelerate AD disease progression, short and long repeats encoding RAN proteins are differentially expressed in AD iPSC-derived cells, and proteasome, autophagy function, β-amyloid and tau pathology are measured.

Proteasome activity in iPSC-derived cells is measured using fluorescent-based assays in which the rate of peptide cleavage by proteasome complexes in protein lysates is determined. Proteasome activity in live cells can also be studied by infecting cells with a GFP expression-vector, and monitoring GFP signal over time. Reduced proteasome function is indicated by reduced fluorescence signal of 7-methylcoumain or high GFP signal in live cells compared with healthy control cells.

To measure autophagy activity, autophagy flux is monitored using a dye that stains autophagosomes. A small molecule-based dye, DALGreen, can also be used to monitor late-phase autophagy. The subcellular location and levels of proteasome and autophagy makers are assayed using immunofluorescence and western blot (e.g., p62, LC3 I/II, proteasome subunits). Increased levels and/or accumulation of p62 has been observed to be linked with autophagy and proteasome inhibition, while the ratio of LC3 I/II has been observed to be associated with autophagy activity, and accumulation of proteasome subunits has been observed to be associated with proteasome stalling and reduced ubiquitin-proteasome activity.

Proteasome and autophagy activity is measured in certain induced cells (e.g., iNeurons, iAstrocytes, iMGL cells, etc.) to examine the cell-type variations. Proteasome and autophagy function are tracked over time, as cells differentiate and mature to provide information regarding when these pathways are affected. Since stress is a risk for neurodegenerative diseases and has been observed to be associated with the increased expression of RAN proteins in C9 ALS/FTD, how proteasome and autophagy are further affected under various stress conditions is tested. RNAseq experiments are conducted to understand the global effects in cells linked with changes in proteasome and autophagy systems.

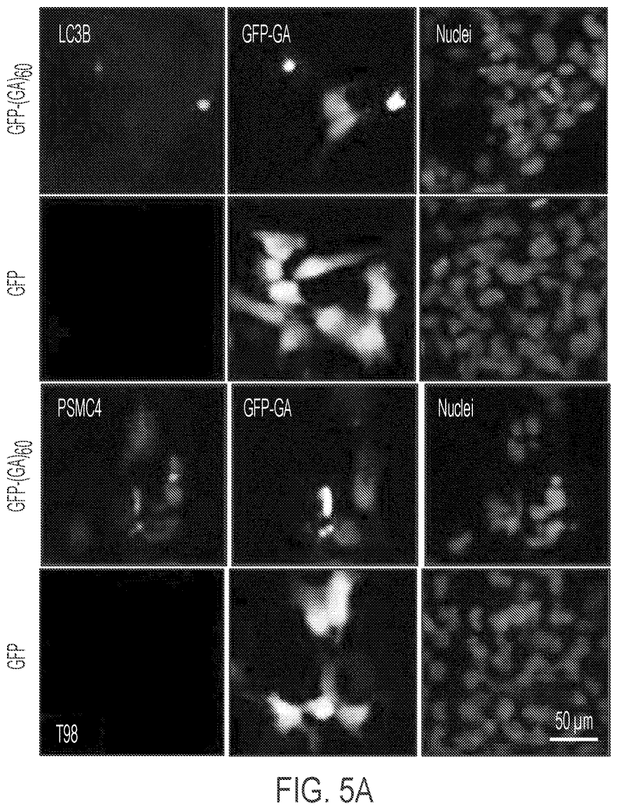

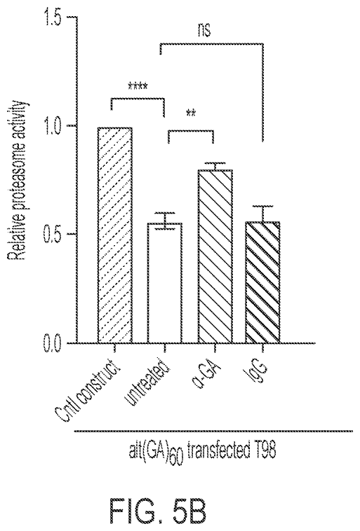

Data indicate that C9 poly(GA) RAN protein aggregates co-localize with autophagy markers LC3B and the 26S proteasome subunit in cultured glioblastoma cells (FIG. 5A). Expression of polyGA RAN proteins was observed to result in a reduction of proteasome activity in HEK293T cells, and these abnormalities are rescued targeting poly(GA) proteins using an α-polyGA antibody (FIG. 5B). These results indicate that GA RAN protein aggregates sequester key proteins involved in proteasome and autophagy systems thereby disrupting the function of these systems.

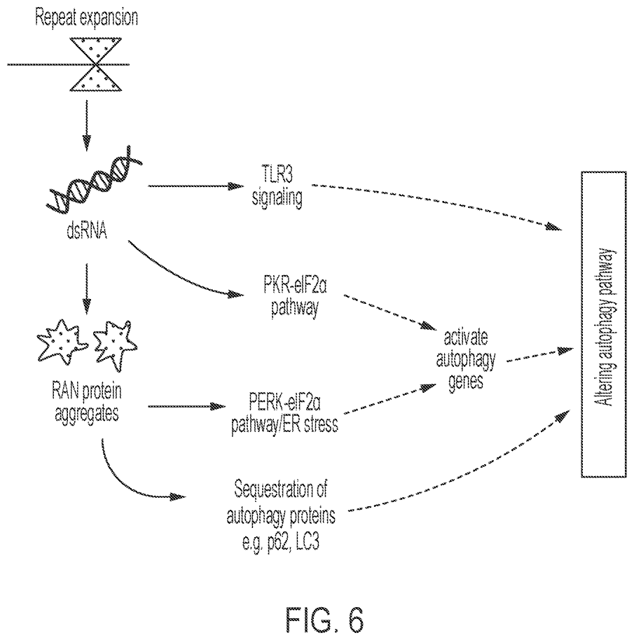

Toll-like receptor 3 (TLR3), a member of the toll-like receptor family that can activate immunological autophagy, has been observed to target double-stranded RNA (dsRNA). Increased eIF2a phosphorylation may result from the expansion RNAs themselves triggering the dsRNA-mediated PKR response or from RAN aggregates triggering ER stress. Monitoring PKR, eIF2a, TLR3, and autophagic flux in the presence of repeat RNA and RAN proteins provides mechanistic insight into autophagy dysfunction and disease progression in repeat expansion disorders (FIG. 6). TLR3-mediated autophagy may also be investigated by knocking down or knocking out TLR3 using siRNA and CRISPR techniques.

RAN protein translation, in some embodiments, leads to dysfunction of proteasome and autophagy pathways, such as decreased activity or improper complex formation, in cells. While proteasome and autophagy dysfunction can be detected in specific cell types, co-culturing neurons with glial cells may accelerate the disruption. Since dsRNA is known to be recognized by TLR3 and to activate eIF2α-PKR pathway, autophagy may be altered early in AD, possibly creating negative feedback that worsens disease over time. Based on the findings that poly(GA) aggregates sequester proteasome and autophagy markers, RAN proteins may inhibit proteasome and autophagy complexes by sequestration into RAN protein aggregates. Additionally, ER stress response due to the accumulation of RAN proteins may, in some embodiments, result in autophagy activation through eIF2α-PERK pathway.

### Example 3

This example describes methods that allow for the isolation of repeat expansion mutations and the identification of locus-specific unique flanking sequences from single DNA samples. This in turn enables direct testing of whether or not a microsatellite repeat expansions contribute to disease in larger groups of patients. Methods described herein, which utilize deactivated clustered regularly interspaced short palindromic repeat associated protein 9 (dCas9), have been observed to pull down microsatellite expansion mutations with repeat motifs containing AGG, TGG, CGG or GGG (NGG) sequences in the protospacer adjacent motif (PAM). This method is referred to herein as Cas9-based repeat enrichment and detection (dCas9READ).

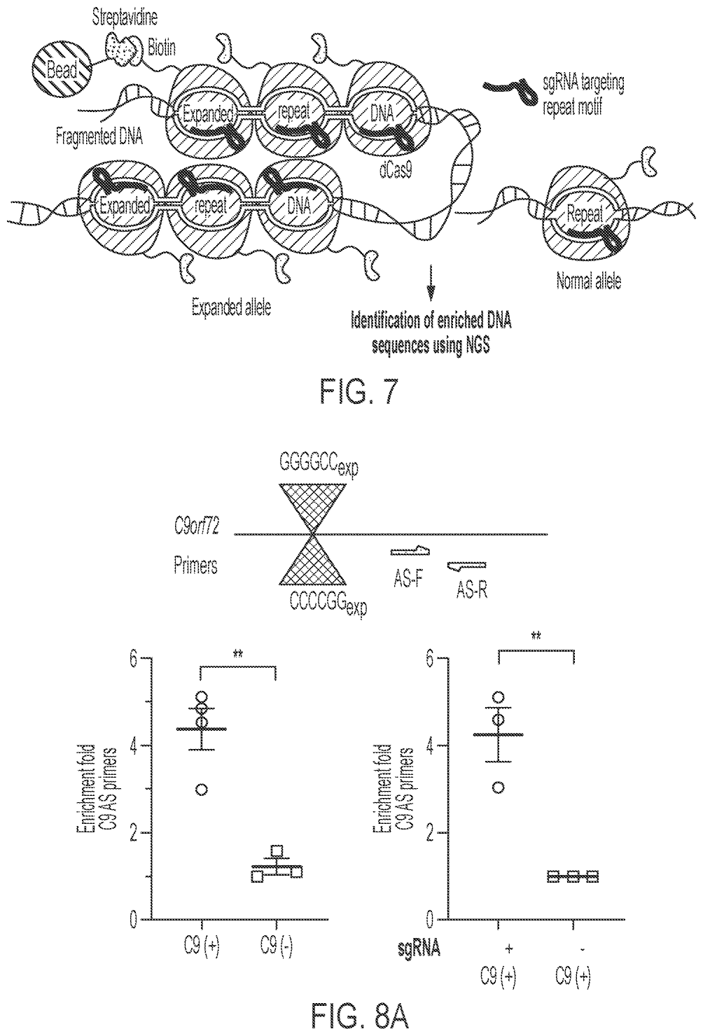

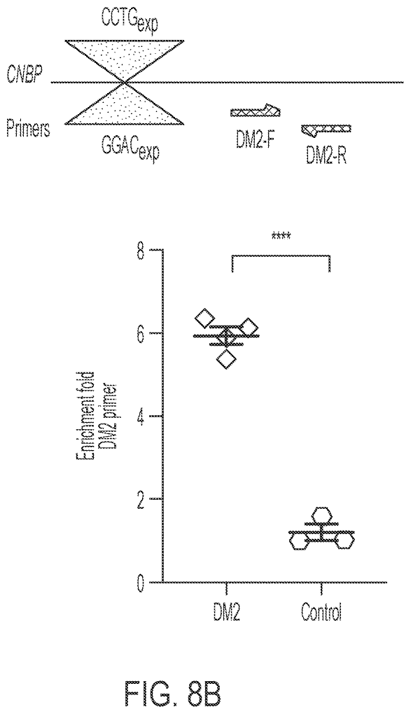

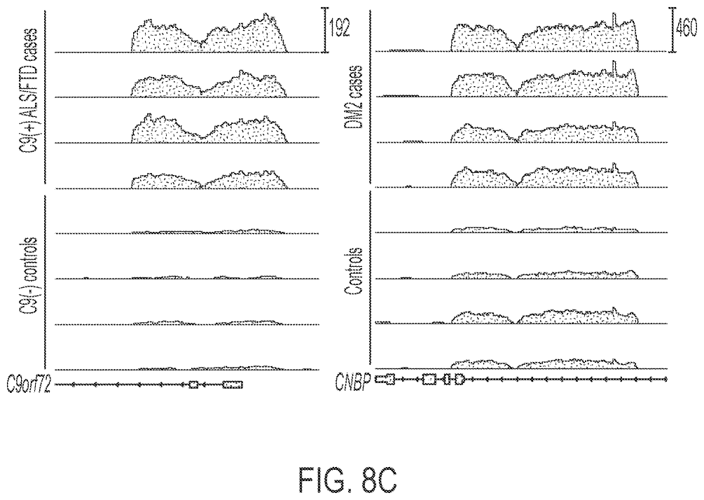

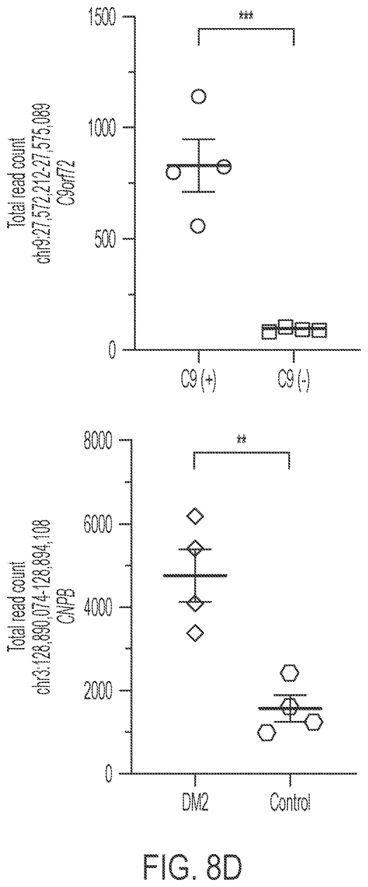

Repeat expansion mutations are difficult to detect using conventional sequencing techniques. Shown herein is a novel assay (dCas9READ) that utilizes sgRNAs and dCas9 to enrich and detect repeat expansions containing NGG protospacer adjacent motifs (PAM) (e.g., GGGGCC in ALS/FTD and CCTG in DM2) and their unique flanking sequences (FIG. 8), as well as non-NGG PAMs. Non-NGG PAM containing repeats include CAG and CTG repeats. The assay as disclosed herein identifies multiple repeat expansions simultaneously, including sequences with non-NGG PAMs, and allow the identification of repeat expansions that are 40-50 repeats longer than the corresponding normal allele. In contrast to the very long non-coding expansions found in DM1, DM2, and ALS/FTD, CAG expansions in the Spinocerebellar ataxias and Huntington's Disease are often only slightly longer (10-100 repeats) than the normal repeat range. Thus, these novel repeat pull-down techniques accelerate the identification of novel expansion mutations, and thus aid is the diagnosis and eventual treatment of RAN protein-associated diseases. The basis of dCas9READ works on the principle that repeat expansion mutations provide more binding sites for single guide RNA (sgRNA)-dCas9 complexes to assemble compared to shorter repeats (FIG. 7). After biotin-streptavidin pull-downs of expansion-containing DNA, next-generation sequencing (NGS) of the expansion enriched genomic DNA (gDNA) was used to identify both the repeat expansion and the corresponding flanking sequences. Identification of the unique flanking sequences and candidate repeat expansion mutations allow PCR and Southern blot testing of specific repeats as putative disease-causing mutations. Compared to conventional pull-down methods, dCas9READ offers rapid binding kinetics and high stability of sgRNA-dCas9-DNA complexes without the need to denature the target DNA.

The dCas9READ protocol was performed using human genomic DNA from C9orf72 ALS/FTD and myotonic dystrophy type 2 (DM2) patients. Using Streptococcus pyogenes dCas9 (spdCas9), it is shown that dCas9READ successfully enriched both C9ALS/FTD G4C2 and the DM2 CCTG repeat expansion DNA 4-6 fold compared to expansion-negative controls (FIG. 8A and FIG. 8B). This enrichment combined with bioinformatics allowed for clear identification of these expansion mutations and their corresponding unique flanking DNA sequences.

An additional control experiment performed without the G4C2 sgRNA showed no similar enrichment of C9 ALS/FTD locus, demonstrating that the pulldown specificity is determined by the repeat containing guide RNA (FIG. 8A). Next-generation sequencing of enriched GGGGCC and CCTG repeats from C9 and DM2 DNA samples showed significant increases in total read counts in samples with expanded C9orf72 and DM2 repeats compared to negative controls (FIG. 8C and FIG. 8D). Importantly, the NGS data reports the enrichment ranking as ratio of total reads of 2-4 kb regions around the selected repeat regions (e.g., G4C2) in enriched vs. unenriched samples showed the C9orf72 locus had the top enrichment score. These data demonstrate dCas9READ can identify repeat expansion loci and the unique flanking sequences surrounding individual repeat expansions without prior knowledge of the location of the expansion mutation in human genome.

### Example 4

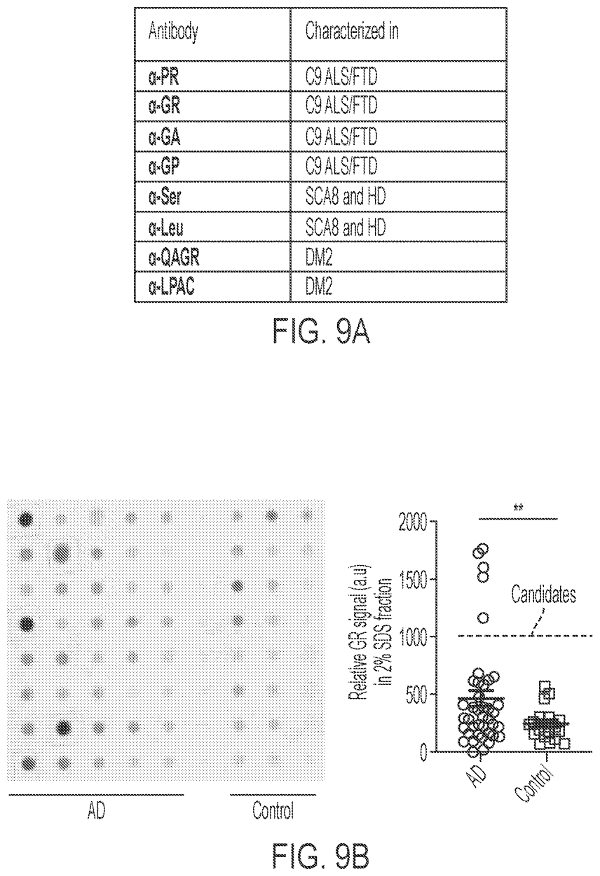

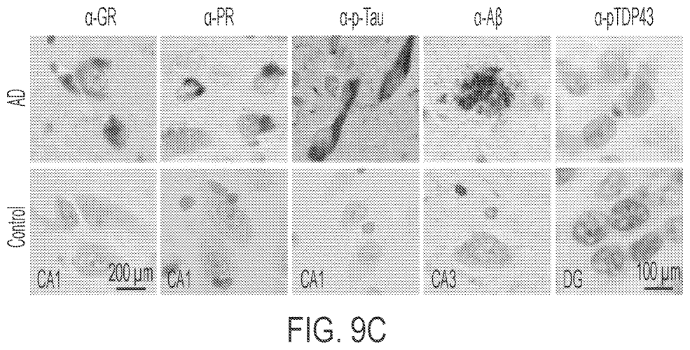

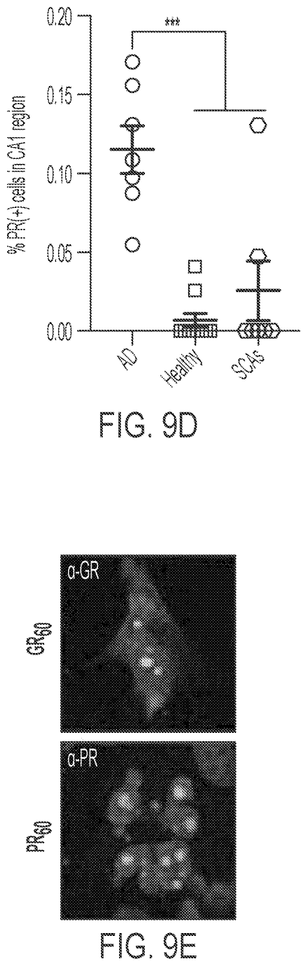

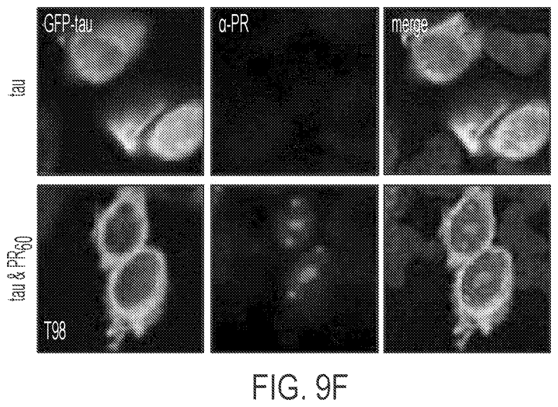

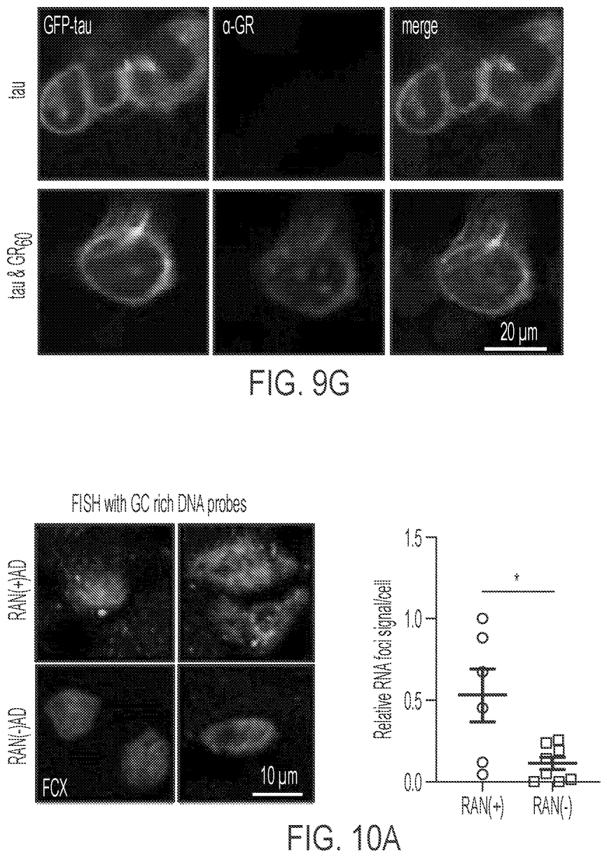

This example describes application of dCas9READ to isolate repeat expansion mutations directly from the genomic DNA of patients with neurodegenerative diseases of unknown genetic etiology. Identifying the flanking sequences at repeat expansion loci allows direct testing of the role of specific expansion mutations in disease. A subset of anti-RAN protein antibodies was used to identify RAN proteins accumulation in AD brains (FIG. 9A). For these experiments, fixed and frozen brain tissue were screened from 120 AD cases with onset of clinical features after the age of 60 (late onset) and 30 age-matched controls. Initial immunoblotting screens showed higher signal in AD vs. control cases using antibodies against glycine-arginine (α-GR) (i.e., anti-GR), proline-arginine (α-PR) (i.e., anti-PR), glycine-alanine (α-GA) (i.e., anti-GA), glycine-proline (α-GP) (i.e., anti-GP), and serine (α-Ser) (i.e., anti-Ser) antibodies. Initial dot-blot screening of insoluble proteins showed positive signals from at least one of these antibodies were found at higher frequency in AD cases (21 of 120 or 17.5%) compared to age-matched controls that did not have neurologic disease (1 versus 30 or 3.3%). A representative dot blot image showing α-GR staining and signal quantification is shown in FIG. 9B.

Immunohistochemical (IHC) studies performed on hippocampal sections from AD cases that were positive for α-GR or α-PR staining by dot blot showed robust perinuclear RAN-positive staining for GR or PR aggregates (FIG. 9C) with no or minimal staining in age-matched healthy or disease controls (e.g., FIG. 9D). The intracellular GR and PR aggregates are not similar to the extracellular Aβ plaques or intracellular p-tau tangle or pTDP43 staining typically found in AD (FIG. 9C). Double-label IHC of p-Tau and PR or GR further demonstrate that the patterns of intracellular accumulation of these RAN proteins and p-tau are distinct. Control IF experiments on cells overexpressing GFP-tau and/or GR₆₀ or PR60 proteins show α-GR and α-PR antibodies recognize their intracellular RAN protein targets (FIG. 9C) but not GFP-tau (FIGS. 9E-9G). These data demonstrate RAN protein staining is present in 17.5% of AD cases in this initial cohort and that RAN proteins accumulate in patterns that distinct from staining typically associated with AD, including Aβ plaques, p-tau tangles, and p-TDP43.

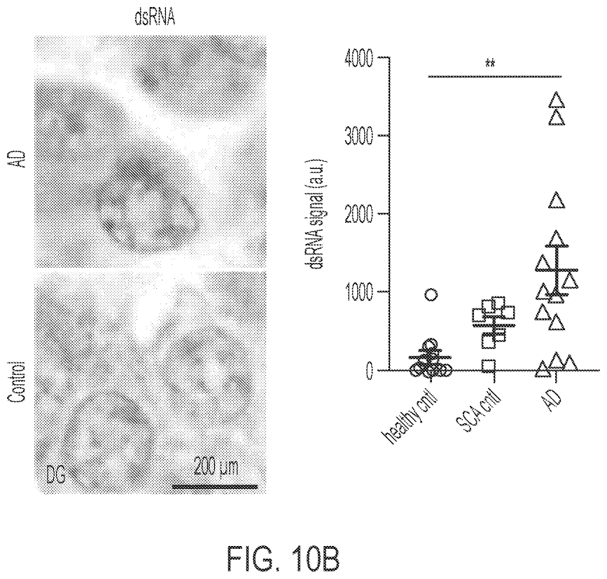

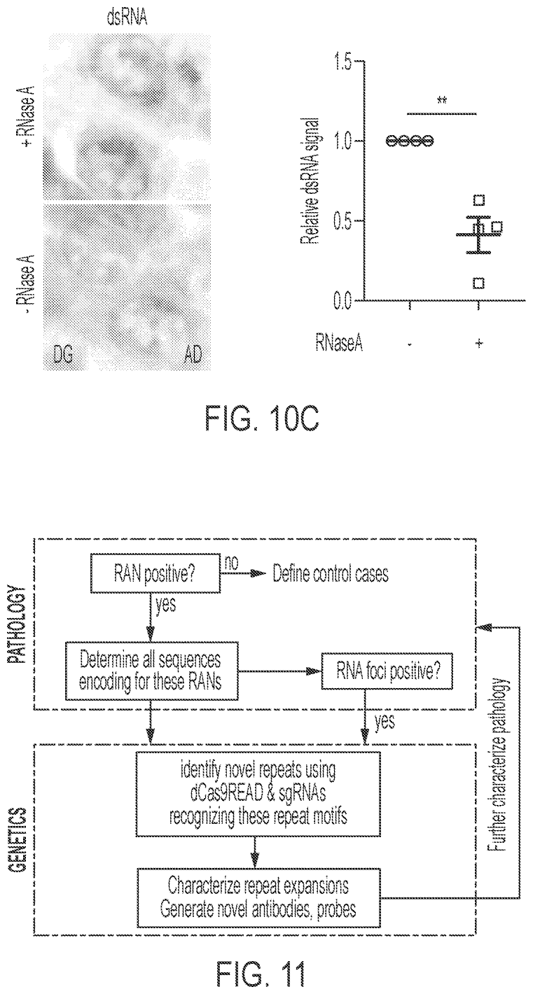

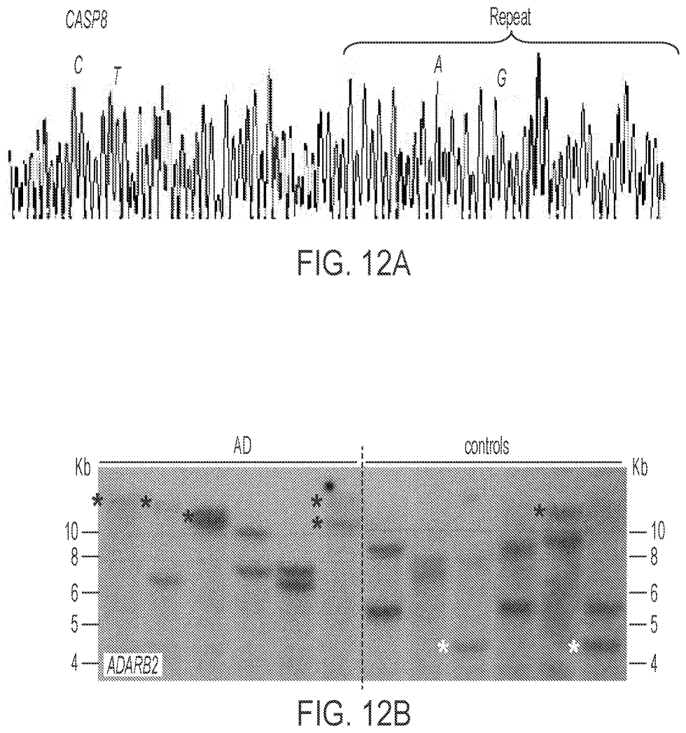

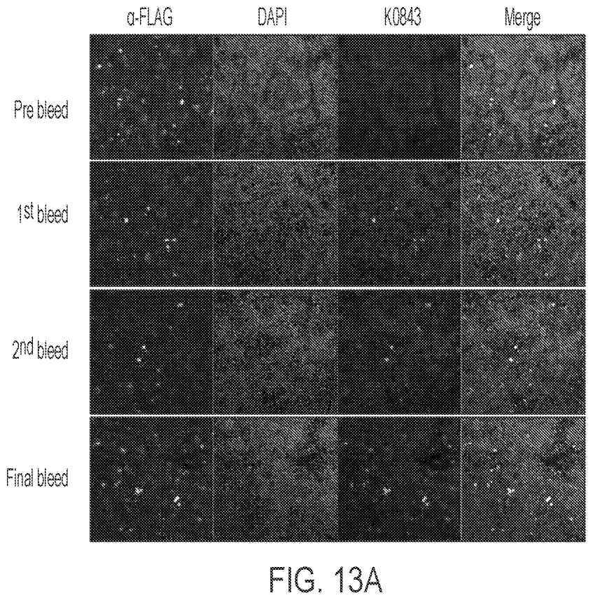

Probes specific for α-GR, α-GA, or α-GP, GC-rich DNA were used to detect repeat expansion RNAs. Fluorescence in situ hybridization (FISH) with CCCCGG (SEQ ID NO: 71) or CCCCGT (SEQ ID NO: 59) probes containing repeats detected punctate RNA foci (FIG. 10A), in a subset of AD cases that showed high signal for α-GR, α-GA, or α-GP antibodies by dot blot screening, but not in AD cases that were negative for these antibodies.

Repeat-containing transcripts produced from repeat expansion mutations can also form secondary structures composed of RNA G-quadruplexes and hairpin structures containing mismatches. To investigate whether dsRNAs accumulate in RAN positive AD cases, the α-dsRNA antibody was used to stain fixed brain tissue. Initial data show increased dsRNA signal in the hippocampus of RAN positive AD cases (FIG. 10B), compared to non-neurologic and disease-controls including a panel of SCAs caused by small CAG repeat expansion mutations. RNAse A treatment significantly reduced α-dsRNA staining, supporting the idea that this antibody specifically stains double-stranded RNAs (FIG. 10C).

The data described in this example highlight methods to identify those repeat expansions that lead to the accumulation of RAN protein aggregates in AD brains (FIG. 11). Antibodies against RAN protein repeat motifs are also used to screen human AD autopsy brains for RAN aggregates. Sequences encoding RAN protein aggregates are used to design fluorescence in situ hybridization (FISH) probes to identify specific RNA foci signals that will provide sequence information to design sgRNAs for repeat enrichment and identification using dCas9READ.

### Example 5

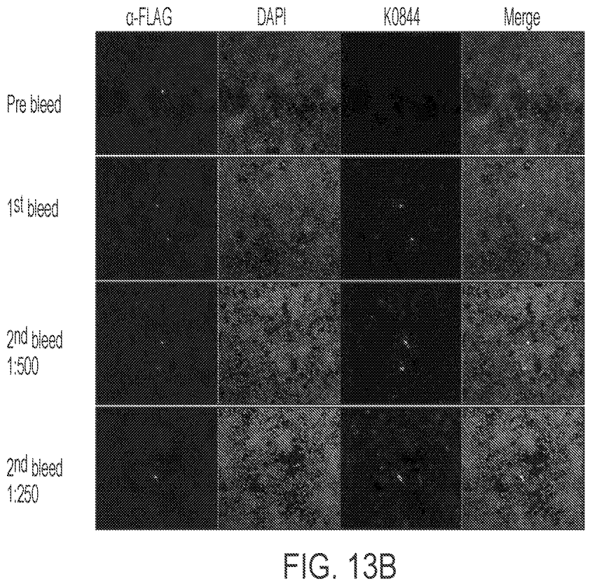

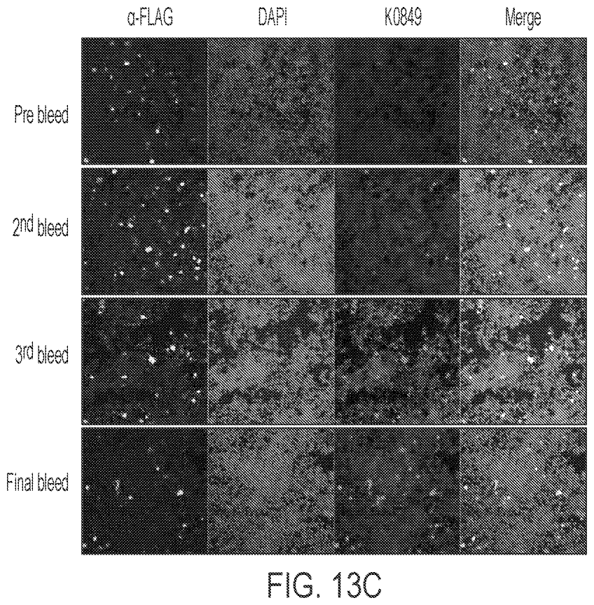

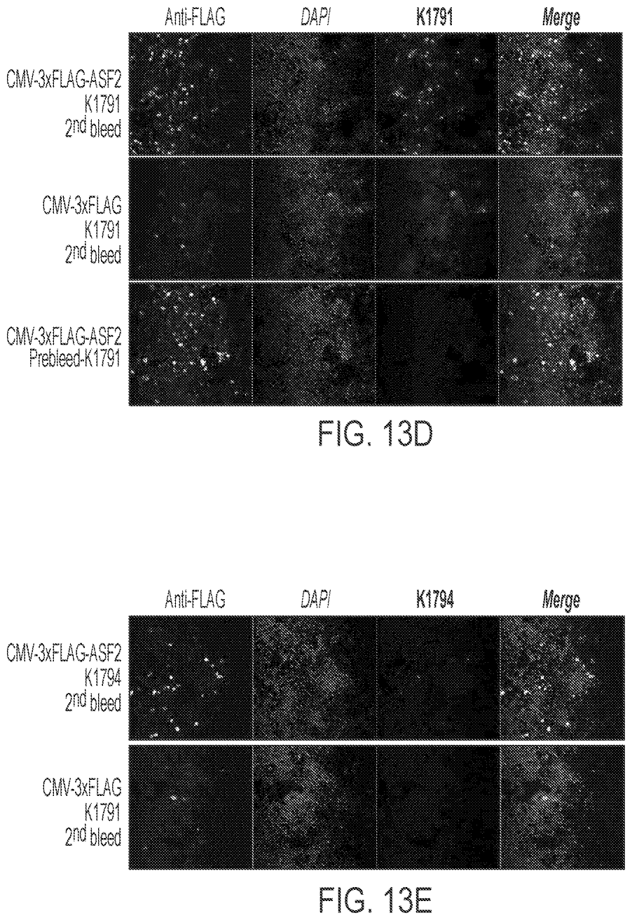

This example describes the generation of anti-RAN antibodies. Antibodies against the RAN protein regions described in Table 9 were produced by injecting a subject with a peptide repeat-containing antigen. Immunofluorescence data validating antibodies in transfected cells expressing recombinant proteins were obtained as shown in FIGS. 13A-13E.

TABLE 9
Antibodies generated against target RAN
protein regions.

| Target | Animal | Titer |
|---|---|---|
| ADARB2- | K1846 | 145,400 |
| poly(GRQRGVNT) | K1861 | 495,400 |
| (SEQ ID NO: 266) | | |
| ADARB2- | K1862 | 1,500,300 |
| poly(GSKHREAE) | K1863 | 1,117,300 |
| (SEQ ID NO: 267) | | |
| polyER | K0842 | 84,200 |
| | K0843 | 444,000 |
| polyEG | K0844 | 114,600 |
| | K0845 | 10,400 |
| polyPL | K0846 | 92,500 |
| | K0847 | 623,100 |
| polyLS | K0848 | 1,500 |
| | K0849 | 63,700 |
| polySP | K0850 | 22,000 |
| | K0851 | 66,400 |
| GAGAGG-F1 | K1785 | 20,700 |
| | K1786 | 97,600 |
| GAGAGG-F2 | K1787 | 142,800 |
| | K1788 | 36,100 |
| GAGAGG-F3 | K1789 | 276,000 |
| | K1790 | 112,000 |
| GAGAGG-ASF1 | K1791 | 365,700 |
| | K1792 | 310,900 |
| GAGAGG-ASF2 | K1793 | 476,400 |
| | K1794 | 913,500 |
| GAGAGG-ASF3 | K1795 | 812,700 |
| | K1796 | 154,300 |

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method of assisting in the diagnosis of a RAN protein-associated disease, the method comprising:
a. performing an assay on a biological sample obtained from a subject to determine whether a RAN protein is present in the biological sample; and
b. identifying the subject as being at risk for a disease associated with RAN protein expression, translation, and/or accumulation if the RAN protein is present in the biological sample.

2. A method for diagnosing a RAN protein-associated disease, the method comprising:
a. detecting in a biological sample obtained from a subject at least one RAN protein; and
b. diagnosing the subject as having the disease based upon the presence of the at least one RAN protein.

3. The method of claim 1 or 2, wherein the RAN protein-associated disease is selected from the group consisting of: amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE).

4. The method of any one of claims 1-3, wherein the RAN protein-associated disease is Alzheimer's Disease (AD).

5. The method of claim 1 or claim 2, wherein the biological sample is blood, serum, or cerebrospinal fluid (CSF).

6. The method of any one of claims 1-5, wherein an antigen retrieval method is performed on the biological sample prior to the detecting.

7. The method of any one of claims 1-6, wherein the RAN protein is poly(CP), poly(GP), poly(Ser), poly(G), poly(GA), poly(GD), poly(GE), poly(GQ), poly(GR), poly(GT), poly(LP), poly(LPAC) (SEQ ID NO: 260), poly(LS), poly(P), poly(PA), poly(PR), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), or poly(PGGRGE) (SEQ ID NO: 258).

8. The method of any one of claims 1-7, wherein the number of poly-amino acid repeats in the at least one RAN protein is greater than or equal to 35.

9. The method of any one of claims 1-7, wherein the number of poly-amino acid repeats in the at least one RAN protein is greater than or equal to 45.

10. The method of any one of claims 1-7, wherein the number of poly-amino acid repeats in the at least one RAN protein is greater than or equal to 50.

11. The method of any one of claims 1-7, wherein the number of poly-amino acid repeats in the at least one RAN protein is greater than or equal to 70.

12. The method of any one of claims 1-11, wherein the detecting is performed by dot blot, 2-D gel electrophoresis, Western Blot, immunohistochemistry (IHC), ELISA, RCA-based ELISA, rtPCR-based ELISA, label free immunoassays such as surface plasmon resonance bio layer interferometry, immunoquantitative PCR, mass spectrometry such as GC-MS, LC-MS, MALDI-TOF-MS, bead based immunoassays, immunoprecipitation, immunostaining, or immunoelectrophoresis.

13. The method of claim 12, wherein the Western blot analysis comprises contacting the sample with an anti-RAN antibody.

14. The method of claim 13, wherein the anti-RAN antibody targets poly(GP), poly(GR), poly(PR), polySer, poly(CP), poly(G), poly(A), poly(GA), poly(GD), poly(GE), poly(GQ), poly(GT), poly(L), poly(LP), poly(LPAC) (SEQ ID NO: 260), poly(LS), poly(P), poly(PA), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), and/or poly(PGGRGE) (SEQ ID NO: 258).

15. The method of claim 13 or claim 14, wherein the anti-RAN antibody targets the C-terminus of a RAN protein.

16. The method of any one of claims 1-15, further comprising administering to the subject a therapeutic for the treatment of a RAN protein-associated disease.

17. The method of claim 16, wherein the therapeutic is an antisense oligonucleotide.

18. The method of claim 17, wherein the antisense oligonucleotide inhibits translation of one or more RAN proteins.

19. A method for treating a RAN protein-associated disease in a subject, the method comprising: administering to a subject a therapeutic for the treatment of the RAN protein-associated disease, wherein the subject has been characterized as having the RAN protein-associated disease by the detection of at least one RAN protein in a biological sample obtained from the subject.

20. The method of claim 19, wherein the RAN protein-associated disease is selected from the group consisting of: amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE).

21. The method of claim 19 or claim 20, wherein the RAN protein-associated disease is Alzheimer's Disease (AD).

22. The method of claim 19, wherein the therapeutic is an antisense oligonucleotide, DNA aptamer, RNA aptamer, or an anti-RAN antibody selected to target the RAN protein detected.

23. The method of claim 22, wherein the anti-RAN antibody targets poly(CP), poly(GP), poly(Ser), poly(G), poly(GA), poly(GD), poly(GE), poly(GQ), poly(GR), poly(GT), poly(LP), poly(LPAC) (SEQ ID NO: 260), poly(LS), poly(P), poly(PA), poly(PR), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), and/or poly(PGGRGE) (SEQ ID NO: 258).

24. The method of claim 22 wherein the anti-RAN antibody targets a C-terminal portion of the RAN protein that comprises an amino acid sequence that is not the repeat amino acid sequences poly(CP), poly(GP), poly(Ser), poly(G), poly(GA), poly(GD), poly(GE), poly(GQ), poly(GR), poly(GT), poly(LP), poly(LPAC) (SEQ ID NO: 260), poly(LS), poly(P), poly(PA), poly(PR), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), and/or poly(PGGRGE) (SEQ ID NO: 258).

25. The method of any one of claims 19-24, wherein the biological sample is blood, serum, or cerebrospinal fluid (CSF).

26. The method of any one of claims 19-25, wherein an antigen retrieval method is performed on the biological sample prior to the detecting.

27. The method of any one of claims 19-26, wherein the RAN protein detected in the biological sample is poly(CP), poly(GP), poly(Ser), poly(G), poly(GA), poly(GD), poly(GE), poly(GQ), poly(GR), poly(GT), poly(LP), poly(LPAC) (SEQ ID NO: 260), poly(LS), poly(P), poly(PA), poly(PR), poly(QAGR) (SEQ ID NO: 261), poly(RE), poly(SP), or poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), and/or poly(PGGRGE) (SEQ ID NO: 258).

28. The method of any one of claims 19-27, wherein the number of poly-amino acid repeats in the at least one RAN protein is greater than or equal to 35.

29. The method of any one of claims 19-28, wherein the detecting is performed by dot blot, 2-D gel electrophoresis, Western Blot, immunohistochemistry (IHC), ELISA, RCA-based ELISA, rtPCR-based ELISA, label free immunoassays such as surface plasmon resonance bio layer interferometry, immunoquantitative PCR, mass spectrometry such as GC-MS, LC-MS, MALDI-TOF-MS, bead based immunoassays, immunoprecipitation, immunostaining, or immunoelectrophoresis.

30. The method of claim 29, wherein the Western blot analysis comprises contacting the sample with an anti-RAN antibody.

31. The method of claim 30, wherein the anti-RAN antibody targets poly(CP), poly(GP), poly(Ser), poly(G), poly(GA), poly(GD), poly(GE), poly(GQ), poly(GR), poly(GT), poly(LP), poly(LPAC) (SEQ ID NO: 260), poly(LS), poly(P), poly(PA), poly(PR), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), or poly(PGGRGE) (SEQ ID NO: 258).

32. The method of claim 30, wherein the anti-RAN antibody targets the C-terminus of a RAN protein that comprises an amino acid sequence that is not the repeat amino acid sequences poly(CP), poly(GP), poly(Ser), poly(G), poly(GA), poly(GD), poly(GE), poly(GQ), poly(GR), poly(GT), poly(LP), poly(LPAC) (SEQ ID NO: 260), poly(LS), poly(P), poly(PA), poly(PR), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), or poly(PGGRGE) (SEQ ID NO: 258).

33. A method for treating a RAN protein-associated disease in a subject, the method comprising: administering to a subject a therapeutic agent for the treatment of the RAN protein-associated disease, wherein the subject has been characterized as having the RAN protein-associated disease by the detection of at least one RAN protein in a biological sample obtained from the subject.

34. The method of claim 33, wherein the RAN protein-associated disease is selected from the group consisting of: amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE).

35. The method of claim 33 or claim 34, wherein the RAN protein-associated disease is Alzheimer's Disease (AD).

36. The method of claim 33, wherein the RAN protein is poly(GR), poly(PR), poly(GP), polySer, poly(CP), poly(G), poly(A), poly(GA), poly(GD), poly(GE), poly(GQ), poly(GT), poly(L), poly(LP), poly(LPAC) (SEQ ID NO: 260), poly(LS), poly(P), poly(PA), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), and/or poly(PGGRGE) (SEQ ID NO: 258).

37. The method of any one of claims 33-36, wherein the at least one RAN protein is encoded by a gene comprising between 2 and 10,000 repeats of a sequence selected from Table 1, Table 2, or Table 3.

38. The method of any one of claims 33-37, wherein the therapeutic agent is a small molecule, interfering nucleic acid, DNA aptamer, RNA aptamer, protein, or antibody.

39. The method of claim 38, wherein the small molecule is an inhibitor of eukaryotic initiation factor 2 (eIF2), eukaryotic initiation factor 3 (eIF3), protein kinase R (PKR), p62, LC3 I subunit, LC3 II subunit, or Toll-like receptor 3 (TLR3).

40. The method of claim 38 or claim 39, wherein the small molecule is metformin or a pharmaceutically acceptable salt, co-crystal, tautomer, stereoisomer, solvate, hydrate, polymorph, isotopically enriched derivative, or prodrug thereof.

41. The method of any one of claims 38-40, wherein the small molecule is buformin, or phenformin.

42. The method of any one of claims 38-41, wherein the small molecule is an inhibitor of TARBP2.

43. The method of claim 38, wherein the interfering nucleic acid is a dsRNA, siRNA, shRNA, miRNA, artificial miRNA (ami-RNA), or antisense oligonucleotide (ASO).

44. The method of claim 38 or claim 43, wherein the interfering nucleic acid inhibits expression of eukaryotic initiation factor 2 (eIF2), eukaryotic initiation factor 3 (eIF3), protein kinase R (PKR), p62, LC3 I subunit, LC3 II subunit, or Toll-like receptor 3 (TLR3).

45. The method of claim 38, claim 43, or claim 44, wherein the interfering nucleic acid inhibits expression of eIF2A.

46. The method of claim 38, claim 43, or claim 44, wherein the interfering nucleic acid inhibits expression of one or more eIF3 subunits selected from the group consisting of eIF3a, eIF3b, eIF3c, eIF3d, eIF3e, eIF3f, eIF3g, eIF3h, eIF3i, eIF3j, eIF3k, eIF31, and eIF3m.

47. The method of claim 38, claim 43, or claim 44, wherein the interfering nucleic acid inhibits expression of protein kinase R (PKR).

48. The method of claim 38, claim 43, or claim 44, wherein the interfering nucleic acid inhibits expression of a gene comprising a nucleic acid repeat comprising the sequence set forth in any one of Tables 1, 2, and 3.

49. The method of claim 48, wherein the interfering nucleic acid binds directly to a sequence set forth in any one of Tables 1, 2, and 3.

50. The method of claim 38, wherein the protein inhibits eukaryotic initiation factor 2 (eIF2), eukaryotic initiation factor 3 (eIF3), protein kinase R (PKR), p62, LC3 I subunit, LC3 II subunit, or Toll-like receptor 3 (TLR3)

51. The method of claim 38 or claim 50, wherein the protein is a dominant-negative variant of protein kinase R (PKR).

52. The method of claim 51, wherein the dominant-negative variant comprises a mutation at amino acid position 296.

53. The method of claim 52, wherein the mutation is K296R.

54. The method of any one of claim 38 or 50-53, wherein the protein is delivered to the subject by a vector.

55. The method of claim 54, wherein the vector is a viral vector, optionally a recombinant adeno-associated virus (rAAV).

56. The method of claim 55, wherein the rAAV comprises an AAV9 capsid protein or variant thereof.

57. The method of claim 38, wherein the antibody targets eukaryotic initiation factor 2 (eIF2), eukaryotic initiation factor 3 (eIF3), protein kinase R (PKR), p62, LC3 I subunit, LC3 II subunit, or Toll-like receptor 3 (TLR3)

58. The method of claim 38 or claim 57, wherein the antibody is an anti-RAN protein antibody.

59. The method of claim 58, wherein the anti-RAN protein antibody targets poly(GR), poly(GP), poly(PR), polySer, poly(CP), poly(G), poly(A), poly(GA), poly(GD), poly(GE), poly(GQ), poly(GT), poly(L), poly(LP), poly(LPAC) (SEQ ID NO: 260), poly(LS), poly(P), poly(PA), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), and/or poly(PGGRGE) (SEQ ID NO: 258) protein.

60. The method of claim 58 or 59, wherein the anti-RAN protein antibody specifically binds to the poly-amino acid repeat of the RAN protein.

61. The method of claim 58 or 59, wherein the anti-RAN protein antibody specifically binds to the C-terminus of the RAN protein.

62. The method of any one of claims 58-61, wherein the anti-RAN protein antibody is a monoclonal antibody.

63. The method of any one of claims 33-62 comprising administering a second therapeutic agent to the subject.

64. The method of claim 63, wherein the second therapeutic agent is selected from donepezil, galantamine, memantine, rivastigimine, or a combination thereof.

65. The method of any one of claims 33-64, wherein the biological sample is blood, serum, or cerebrospinal fluid (CSF).

66. The method of any one of claims 33-65, wherein the detection comprises a binding assay, hybridization assay, immunoblot analysis, Western blot analysis, immunohistochemistry, and/or ELISA, optionally wherein the ELISA is RCA-based ELISA or rtPCR-based ELISA.

67. The method of claim 66, wherein the hybridization assay comprises Fluorescence In Situ Hybridization (FISH) and/or dCas9-based enrichment.

68. The method of claim 67, wherein FISH is optionally performed with CCCCGG (SEQ ID NO: 71) or CCCCGT (SEQ ID NO: 59) probes containing repeats.

69. The method of claim 67, wherein the dCas9-based enrichment is performed using a Streptococcus pyogenes dCas9 (spdCas9).

70. The method of claim 67 or claim 69, wherein the dCas9-based enrichment is performed using a Cas9 protein that is a mutant of a wild-type Cas9.

71. The method of any one of claim 67 or 69-70, wherein the dCas9-based enrichment is performed using a Cas9 protein that comprises a mutation that inactivates a Cas9 nuclease activity.

72. The method of any one of claim 67 or 69-71, wherein the dCas9 protein comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis dCas9, a Campylobacter lari dCas9, or a Streptococcus thermophilus dCas9.

73. The method of any one of claims 66-72, wherein the detection further comprises nucleic acid sequencing, optionally wherein the sequencing is Next-Generation Sequencing (NGS).

74. A method for diagnosing Alzheimer's disease, the method comprising:
(i) detecting in a sample obtained from a subject at least one RAN protein;
(ii) determining that the at least one RAN protein is not transcribed from a C9orf72 locus of the subject; and
(iii) diagnosing the subject as having Alzheimer's disease based on the presence of the at least one RAN protein that was not transcribed from the C9orf72 locus.

75. The method of claim 74, wherein the sample is central nervous system (CNS) tissue, blood, or cerebrospinal fluid (CSF).

76. The method of claim 74 or 75, wherein the detecting comprises contacting the sample with an anti-RAN antibody.

77. The method of claim 76, wherein the anti-RAN protein antibody targets poly(GR), poly(PR), poly(GP), polySer, poly(CP), poly(G), poly(A), poly(GA), poly(GD), poly(GE), poly(GQ), poly(GT), poly(L), poly(LP), poly(LPAC) (SEQ ID NO: 260), poly(LS), poly(P), poly(PA), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), and/or poly(PGGRGE) (SEQ ID NO: 258) protein.

78. The method of claim 76 or claim 77, wherein the anti-RAN antibody is the antibody of any one of claims 89-96.

79. The method of any one of claims 74-78, wherein the detecting comprises performing dCas9-based enrichment on the sample.

80. The method of claim 79, wherein the dCas9 protein is Streptococcus pyogenes dCas9 (spdCas9).

81. The method of claim 79, wherein the dCas9 protein comprises a Staphylococcus aureus dCas9, a Streptococcus pyogenes dCas9, a Campylobacter jejuni dCas9, a Corynebacterium diphtheria dCas9, a Eubacterium ventriosum dCas9, a Streptococcus pasteurianus dCas9, a Lactobacillus farciminis dCas9, a Sphaerochaeta globus dCas9, an Azospirillum dCas9, a Gluconacetobacter diazotrophicus dCas9, a Neisseria cinerea dCas9, a Roseburia intestinalis dCas9, a Parvibaculum lavamentivorans dCas9, a Nitratifractor salsuginis dCas9, a Campylobacter lari dCas9, or a Streptococcus thermophilus dCas9.

82. The method of any one of claims 74-81, wherein the detecting further comprises nucleic acid sequencing, optionally wherein the sequencing is Next-Generation Sequencing (NGS).

83. A method for increasing proteasome activity in a cell, the method comprising administering to the cell an anti-RAN protein antibody in an amount sufficient to reduce RAN protein aggregation in the cell.

84. The method of claim 83, wherein the cell is a neuronal cell, astrocyte, or glial cell.

85. The method of claim 83 or 84, wherein the cell contains a gene having a nucleic acid sequence comprising at least 35 repeats of a sequence set forth in any one of Tables 1, 2, and 3.

86. The method of any one of claims 83-85, wherein the anti-RAN protein antibody is a monoclonal antibody.

87. The method of any one of claims 83-86, wherein the administration of the anti-RAN protein antibody results in an increase in proteasome activity in the cell relative to proteasome activity in the cell prior to the administration.

88. The method of claim 87, wherein the increase in proteasome activity is indicated by a decrease in P62 subunit expression or activity in the cell.

89. An antibody or antigen-binding fragment that specifically binds to any one or more of polySer, poly(GP), poly(PR), poly(GR), poly(CP), poly(G), poly(A), poly(GA), poly(GD), poly(GE), poly(GQ), poly(GT), poly(L), poly(LP), poly(LPAC) (SEQ ID NO: 260), poly(LS), poly(P), poly(PA), poly(QAGR) (SEQ ID NO: 261), poly(RE), poly(SP), poly(VP), poly(FP), poly(GK), poly(FTPLSLPV) (SEQ ID NO: 262), poly(LLPSPSRC) (SEQ ID NO: 263), poly(YSPLPPGV) (SEQ ID NO: 264), poly(HREGEGSK) (SEQ ID NO: 255), poly(TGRERGVN) (SEQ ID NO: 265), and/or poly(PGGRGE) (SEQ ID NO: 258).

90. An antibody or antigen-binding fragment thereof that specifically binds to a RAN protein, wherein the antibody or antigen-binding fragment comprises a heavy chain variable region (VH) comprising:
(i) a CDR1 region comprising an amino acid sequence selected from the group consisting of SEQ ID NOs: 117, 119, 121 and 123;
(ii) a CDR2 region comprising an amino acid sequence selected from the group consisting of SEQ ID NOs: 125, 127, 129 and 131; and/or
(iii) a CDR3 region comprising an amino acid sequence selected from the group consisting of SEQ ID NOs: 133, 135, 137 and 139.

91. An antibody or antigen-binding fragment thereof that specifically binds to a RAN protein, wherein the antibody or antigen-binding fragment comprises a light chain variable region (VL) comprising:
(i) a CDR1 region comprising an amino acid sequence selected from the group consisting of SEQ ID NOs: 118, 120, 122 and 124;
(ii) a CDR2 region comprising an amino acid sequence selected from the group consisting of SEQ ID NOs: 126, 128, 130 and 132; and/or
(iii) a CDR3 region comprising an amino acid sequence selected from the group consisting of SEQ ID NOs: 134, 136, 138 and 140.

92. The antibody or antigen-binding fragment thereof of any one of claims 89-91, wherein the antibody or antigen-binding fragment comprises a variable heavy chain amino acid sequence as set forth in SEQ ID NOs: 109, 111, 113 or 115.

93. The antibody or antigen-binding fragment thereof of any of claims 89-92, wherein the antibody or antigen-binding fragment comprises a variable light chain amino acid sequence as set forth in SEQ ID NOs: 110, 112, 114 or 116.

94. The antibody or antigen-binding fragment thereof of any of claims 89-93, wherein the antibody binds polyGA.

95. The antibody or antigen-binding fragment thereof of any of claims 89-93, wherein the antibody binds polySer.

96. The antibody or antigen-binding fragment thereof of any of claims 89-93, wherein the antibody binds polyPR.

97. A composition comprising the antibody or antigen-binding fragment thereof of any one of claims 89-96 and a pharmaceutically acceptable carrier and/or a pharmaceutically acceptable buffer.

98. The composition of claim 97, for use in treating a repeat expansion disease.

99. The composition of claims 97 and 98, wherein the repeat expansion disease is amyotrophic lateral sclerosis (ALS), or frontotemporal dementia; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS); Fuch's endothelial corneal dystrophy (FECD); Huntington's disease-like 2 syndrome (HDL2); Fragile X syndrome (FXS); disorders related to 7p1 1.2 folate-sensitive fragile site FRA7A; disorders related to folate-sensitive fragile site 2q1 1 FRA2A; and Fragile XE syndrome (FRAXE).

100. The composition of claims 97-99, wherein the repeat expansion disease is Alzheimer's Disease (AD)

101. The composition of claim 97, for use in treating Alzheimer's disease.

102. An isolated nucleic acid molecule encoding the antibody or antigen-binding fragment thereof of any one of claims 89-96.

103. A cell transformed with a nucleic acid of claim 102.

104. The cell of claim 103, wherein said cell is a mammalian cell.

105. The cell of claim 103 or claim 104, wherein said cell is a cell is a human cell.

106. A method of treating Alzheimer's disease in a subject, the method comprising: administering the antibody or antigen-binding fragment of any of claims 89-96, wherein the subject has been characterized as having Alzheimer's disease by the detection of at least one RAN protein in a biological sample obtained from the subject.

107. A method of treating repeat expansion disease in a subject, the method comprising: administering the antibody or antigen-binding fragment of any of claims 89-96, wherein the subject has been characterized as having a repeat expansion disease by the detection of at least one RAN protein in a biological sample obtained from the subject.
